(19) United States
(12) Patent Application Publication
(10) Pub. No.: US 2025/0264457 A1
(43) Pub. Date: Aug. 21, 2025

(54) A BIOMARKER AND COMPOSITIONS TO INCREASE THE THERAPEUTIC INDEX OF NEOADJUVANT IMMUNOTHERAPY IN MUSCLE-INVASIVE UROTHELIAL CARCINOMA

(71) Applicants: INSTITUT GUSTAVE ROUSSY, VILLEJUIF (FR); INSERM (INSTITUT NATIONAL DE LA SANTE ET DE LA RECHERCHE MEDICALE), PARIS (FR); UNIVERSITE PARIS- SACLAY, GIF-SUR-YVETTE (FR)

(72) Inventors: Anne-Gaelle GOUBET, VEVEY (CH); Laurence ZITVOGEL, PARIS (FR); Yohann LORIOT, VILLE-BON-SUR-YVETTE (FR)

(21) Appl. No.: 18/839,356

(22) PCT Filed: Feb. 16, 2023
(86) PCT No.: PCT/EP2023/053977

§ 371 Date: Aug. 16, 2024

(30) Foreign Application Priority Data
Feb. 17, 2022 (EP) 22305181.4

(51) Int. Cl. G01N 33/50

(52) U.S. Cl. CPC G01N 33/505; G01N 33/5023

(57) ABSTRACT

The present invention relates to the treatment of locally advanced and metastatic muscle-invasive bladder cancers (MIBC). The present invention provides compositions to be used in immunotherapy of MIBC, especially in combination with an anti-PD-1/PD-L1/PD-L2 antibody-based therapy. The present invention also provides biomarkers of response to anti-PD-1/PD-L1/PD-L2 immune checkpoint blocking antibodies (ICBs), alone or together with anti-CTLA4 antibodies and/or chemotherapy, for best guiding their neoadjuvant use and avoid unefficient administration of potentially toxic drugs to patients with localized bladder cancers.

### FIELD OF THE INVENTION

The present invention relates to the field of anticancer immunotherapy. In particular, the present invention concerns the treatment of locally advanced and metastatic muscle-invasive bladder cancers (MIBC).

The aim of the present invention is to increase the therapeutic index of neoadjuvant immunotherapy in muscle-invasive urothelial carcinoma, by providing compositions which can synergize with this immunotherapy as well as theranostic tools to avoid unefficient treatments.

### BACKGROUND OF THE INVENTION

Programmed cell death protein 1 (PD-1) is an immune checkpoint protein expressed on T cells (Tumeh et al. 2014). PD-1 inhibits T cell responses to cancer after binding to one of its ligands, programmed cell death ligand 1 (PD-L1, B7-H1, CD274) or PD-L2 (B7-DC, CD273) (Ishida et al. 1992; Pardoll 2012). Therapeutic blockade of PD-1 or PD-L1 with monoclonal antibodies (mAbs) leads to durable tumor regressions in patients with several cancer types (Hamid et al. 2013; Herbst et al. 2014; Topalian et al. 2014; Tumeh et al. 2014). These clinical observations have spurred the FDA approval of two anti-PD-1 antibodies, pembrolizumab (MK-3475) and nivolumab (BMS-936558), for the treatment of localized and metastatic cancers across numerous indications.

Pembrolizumab targets epitopes on the PD-1 molecule with high affinity and specificity. This mAb is of the IgG4 subclass and hence has low affinity for C1q and Fc receptors (Scapin et al. 2015). Pharmacokinetics studies based on flow cytometric methods (Patnaik et al. 2015) indicated a dose-independent high receptor occupancy at the peak and a plateau reached at 60 days (Patnaik et al. 2015). Preclinical studies in immunodeficient NOD/SCID/γ_{c}^{null} mice transplanted with human CD34⁺ hematopoietic stem cells and inoculated with partially histocompatible patient-derived xenografts unveiled that pembrolizumab stimulated anticancer T cell responses (Wang et al. 2018). Following PD-1 blockade, both CD4⁺ and CD8⁺ T cells expanded in blood, but CD8⁺ T cells were mandatory for the antitumor efficacy of pembrolizumab and migrated from tumor margins to nests (Wang et al. 2018). In patients, pembrolizumab promotes minor changes in blood while mobilizing not only T cells but also B cells and suppressive monocytes in tumors (Ribas et al. 2016). However, expansion of effector memory CD8⁺ tumor-infiltrating lymphocytes (TIL) has been recognized as the best pharmacodynamic hallmark of bioactivity of this therapeutic mAbs against melanoma (Ribas et al. 2016).

Urothelial carcinoma (UC) is a highly prevalent malignancy that remains difficult to treat in case of muscle-invasive tumors (Jordan et Meeks 2019). Non-metastatic muscle-invasive bladder cancers (MIBC) are mainly treated with cisplatin-based neoadjuvant chemotherapy followed by radical cystectomy and harbor a poor prognosis with a five-year survival of 60% (Alfred Witjes et al. 2017). However, a significant subset of patients is ineligible for neoadjuvant cisplatin-based chemotherapy. Since 2017, ICBs, such as anti-PD-1/PD-L1 antibodies, have emerged as a successful immunotherapeutic strategy for advanced and metastatic UC (Balar, Castellano, et al. 2017; Balar, Galsky, et al. 2017; Bellmunt et al. 2017; Powles et al. 2018; 2020; Sharma et al. 2017). Driven by the promising results in advanced diseases, ICBs indications have been considered for the treatment of lower-stage bladder tumors, including non-MIBC and non-metastatic MIBC (NCT02844816, NCT02901548 and NCT02792192) (Bajorin et al. 2021; Balar et al. 2021). To our knowledge, four neoadjuvant trials investigating either anti-PD-1/PD-L1 monotherapy or the combination of anti-CTLA4 and anti-PD-1/PD-L1 mAbs before cystectomy have been reported for cisplatin-ineligible patients or in patients who declined neoadjuvant chemotherapy (Gao et al. 2020; Necchi et al. 2018; Powles et al. 2019; van Dijk et al. 2020). Pathological complete response (pCR) defined as the absence of tumor cells (ypT0N0) was observed in 31% to 46%. Substantial toxicity (grade 3 immune related adverse events) was reported in 11% and from 21 to 55% of patients treated with anti-PD-1/PD-L1- or combined ICB, respectively. Therefore, there is a need for biomarker discovery and appropriate co-treatment to increase the therapeutic index of neoadjuvant immunotherapy in MIBC.

### SUMMARY OF THE INVENTION

According to a first aspect, the present invention pertains to the use of an immunogenic composition comprising antigens from Escherichia coli (E. coli), preferably uropathogenic E. coli (UPEC), Staphylococcus capitis (S. capitis) and/or Bacillus Calmette-Guérin (BCG), for treating muscle-invasive bladder cancer (MIBC).

Such a composition can comprise live or dead bacteria, fragments thereof or isolated protein(s) or peptide(s), or a nucleic acid encoding bacterial proteins or peptides, and can be administered by any route appropriate to elicit an immune response against said bacteria. According to a particular embodiment, the composition is administered in combination with an immune checkpoint inhibiting treatment, such as a monoclonal antibody targeting PD1, PD-L1 or PD-L2, alone or combined with an anti-CTLA4 antibody.

The invention also pertains to the use of an antibody targeting an antigen from E. coli or from S. capitis for treating MIBC, as well as to an antibody-drug conjugate (ADC) comprising such an antibody.

Another aspect of the present invention, described in Examples 6 and 10, is a theranostic method for determining if a patient having a MIBC or a kidney cancer is likely to respond to an anti-PD1/PD-L1/PD-L2 Ab-based therapy, comprising a step of assessing, in a sample from said patient:
(i) the level of E. coli-specific IgG,
(ii) the level of S. capitis-specific IgG and IgA,
(iii) the level of BCG-specific IgG,
(iv) the level of E. coli-specific CXCL13 producing follicular helper CD4⁺ T (T_{FH}), and/or
(v) the level of cytokine release during incubation of whole blood with E. coli,
   wherein if said level(s) is(are) superior to (a) predetermined threshold(s), the individual is likely to respond to said anti-PD1/PD-L1/PD-L2 Ab-based therapy.

The invention also relates to theranostic methods for assessing whether an MIBC patient treated with an anti-PD1/PD-L1/PD-L2 Ab-based therapy responds to the treatment, in order to avoid unefficient and potentially toxic further administrations in case of failure. Such a method is based on the comparison of the serum level(s) of CXCL13 and/or CCL19 in said patient before and after administration of said anti-PD1/PD-L1/PD-L2 Ab-based therapy, wherein an increase of CXCL13 and/or CCL19 three weeks after the first administration of said anti-PD1/PD-L1/PD-L2 Ab-based therapy indicates that the individual responds to the therapy.

Another method for assessing the patient's response is based on immunostaining of samples corresponding to representative slides of TURBT and cystectomies with antibodies directed against IgG4, PD1, CD4 and CD38, wherein if co-stained cells are observed, the individual responds to the treatment, and if no co-stained cells are observed, the treatment can be stopped.

### BRIEF DESCRIPTION OF THE DRAWINGS

FIG. 1. PANDORE patient characteristics and survival post-neoadjuvant pembrolizumab. Kaplan Meier survival curves for progression-free survival (left panel) and overall survival (right panel) in the efficacy population (black) and according to the pathological response (no response: orange, partial response: light blue and complete response: green) in Cox regression analyses.

FIG. 2. Blood TFH and ASC are associated with progression-free survival (PFS) in PANDORE patients. A-B. Spearman correlation matrices between histopathological features before (left) and after (right) pembrolizumab in bladder tissues. Significant correlations are indicated with a star. GC: germinal centers, LA: lymphoid aggregates, RF: resting follicles. Detailed results for the correlations between CD8+ and CD4+PD-1+ TILs (left) or tumoral tertiary lymphoid structures (TLS) like (right), at baseline (blue) and after treatment (pink). Each dot represents one patient and R and p values are indicated (B). C. Kaplan-Meier curves and Cox regression analysis of progression-free survival (PFS) according to TLS-like using medians as cutoff values, at baseline (left panel) and after treatment (right panel). Statistical analyses used Log-rank test. D-E. Fold ratios in log between mean values of each PD-1+ T cell subset (D) or B cells (E) between non progressors versus progressors (upper panels) and P-values of log-rank tests (lower panels) at baseline (D) or after 3 cycles of pembrolizumab (E, also refer to FIG. 9E-F) assessing the contribution of each CD4+ T cell subset or B cells (E) to PFS. CD4+ T cell subsets were evaluated using mass cytometry (n=30 samples), clustering method (PhenoGraph) followed by manual annotation and manual gating. B cells subsets were evaluated using mass cytometry (n=29 samples) followed by manual gating. F-G. Kaplan-Meier curves and Cox regression analysis of PFS according to circulating TFH TCM (left panel) and CD38+ TFH TCM (right panel) in CD4+ T cells at baseline (F) or circulating antibody-secreting cells (ASC) in CD45+ cells after 3 cycles of pembrolizumab (G), with medians as cutoff values. Statistical analysis used Log-rank test.

FIG. 3. Tumoral TFH-like CD4+ T cells are associated with TLS-like and predict PFS. A. Accumulation of CD4+CD38+PD-1+ in TLS-like compared to whole tissue before treatment (refer to Materials &Methods). Data are shown as means±SEM. Each dot represents one patient and color code indicates the progression-free survival (PFS) status at 24 months. Statistical analysis used paired t-tests (Mann-Whitney U-test). B. Spearman correlations between CD4+CD38+PD-1+ cells and TLS-like in bladder tissues, at baseline (top panel) and after 3 cycles of pembrolizumab (bottom panel). Each dot represents one patient. C. Kaplan-Meier curves and Cox regression analysis of PFS according to CD4+CD38+PD-1+ cells in TLS-like, at baseline in TURBT, using medians as cutoff values. Statistical analyses used Log-rank test. D. Single cell transcriptomics of CD45+ in 4 radical cystectomy specimens and 2 PBMCs samples. Analysis of Rhapsody single-cell RNA-seq data, displayed by UMAP (left panel). Seurat clustering of 2,432 CD45+ cells from primary bladder tumors, surrounding non tumoral tissue and PBMCs. Expression levels of the CXCL13 gene product in CD4+ TILs (cluster 2) depicted in violin plots as well as of PD-1, CD38 and CD28 at protein level in 3 specimens collected post-anti-PD-1 mAbs (healthy bladder tissue from complete responder (P #38), healthy bladder tissue and bladder cancer from non-responder (P #02)). E. Multiplex array of inflammatory analytes in plasma. Volcano plots showing differential plasma concentrations of cytokines/chemokines before and after one injection of pembrolizumab, in the group of patients experiencing or not a relapse and/or death before 24 months (left and right panel, respectively). Volcano plots was generated computing for each soluble factor: (i) the log 2 of fold ratio among the mean values after normalization, after 1st injection versus pre-injection of pembrolizumab (x axis); (ii) the log 10 of P-values deriving from paired Wilcoxon test calculated in absolute values (y axis). Black and colored dots are considered non-significant (p<0.05) or significant (p>0.05), respectively. F. Plasma concentration of CXCL13 (log 10) in pre-versus post-1st injection of pembrolizumab in the 2 groups (according to PFS status at 24 months). Each line represents one patient. The dotted line indicates the lower detection threshold of CXCL13. Statistical analysis used paired t-test (Mann-Whitney U-test).

FIG. 4. Pre-existing TFH-like CD4+ T cells are prerequisites for a functional response of MIBC to pembrolizumab ex vivo. A. Overview of the in situ/in vitro (in sitro) test. B. Bar chart showing log-ratio of CD4+ TILs cluster values between immuno-reactive versus non-immuno-reactive MIBC (top panel). Increasing blue gradient for increased fold ratios. Bar charts showing the P-values of unpaired t-test (Mann-Whitney U-test) for each CD4+ TILs cluster (bottom panel). C. Expression levels of the CXCL13 gene product in CD4+ TILs (cluster 2, as shown in FIG. 3D) depicted in violin plots as well as PD-1, CD38 and CD28 at protein level in 2 specimens collected before the in sitro assay. Samples from untreated patients were utilized (B #11: immunoreactive tumor, B #12: immune-resistant tumor). D. Percentages of CD38highCD28+PD-1high (cluster 21) in CD4+ TILs (left panel), quantification of CXCL13 production after 3 days of in sitro assay without treatment (middle panel) and Spearman correlation between both parameters (right panel). Box plots display a group of numerical data through their 3rd and 1st quartiles (box), mean (central band), minimum and maximum (whiskers). Each dot represents one tumor. Statistical analyses used unpaired t-test (Mann-Whitney U-test).

FIG. 5. Preferential binding of pembrolizumab to TFH and CD38+CD28+ TFH like cells. A. Unbiased analyses of IgG4+PD-1+ within each cluster of peripheral CD4+ T cell subset. For each cluster, the average of mean values at 3 time points for all patients (post-1st, post-2nd, post-3rd) as well as the standard error of means are depicted. B. Frequencies of IgG4+PD-1+ within activated (CD38+) Central Memory effector T cells (TCM) and effector memory T cells (TEM) and TFH effector T cells (TE)-TEM-TCM as well as in naïve T cells as negative control. C. Frequencies of IgG4+PD-1+ in activated CD38+ TEM before the 3rd injection of pembrolizumab in the group of patients experiencing PFS < or ≥24 months. Box plots display a group of numerical data through their 3rd and 1st quartiles (box), mean (central band), minimum and maximum (whiskers). Each dot represents one patient. Statistical analysis used unpaired t-test (Mann-Whitney U-test). D. Accumulation of CD4+CD38+IgG4+ TFH-like in TLS-like compared to whole tissue, after treatment (refer to M&M). Box plots display a group of numerical data through their 3rd and 1st quartiles (box), mean (central band), minimum and maximum (whiskers). Each dot represents one patient. Statistical analysis used paired t-tests (Mann-Whitney U-test).

FIG. 6. MIBC contain Gram negative bacteria with uropathogenic capacities. A. Representative photograph of bladder showing non-tumoral urothelium (above discontinuous line) with invasive cells of urothelial carcinoma containing granular cytoplasmic structures (arrows) in patient #043, in HES at 20× magnitude (left panel). LPS immunohistochemical staining of the same area highlighting granular cytoplasmic positivity in the normal urothelium, immune cells (grey arrow) and tumor cells (black arrow), at 20× magnitude. B. Proportions of patients with positive or negative LPS staining in immune cells (IC) and tumor cells (n=33 patients). C. Enumeration of granular cytoplasmic structures in normal and tumoral urothelium. Each dot represents one patients' sample. Statistical analysis used unpaired t-tests (Mann-Whitney U-test). D. Stacked bar chart showing bacteria cultivated by culturomics (refer to M&M) within tumor and non-tumor tissues (n=6 patients with paired tissues). Total number of positive (black) or negative (grey) samples is depicted. E. Proportions of samples with positive or negative PCR detection of Escherichia coli (n=20 bladder specimens, with n=9/20 post-pembrolizumab and n=11/20 untreated). F. Proportions of patients with paired samples of tumor and surrounding non-tumor tissues (n=6 untreated patients) for PCR detection of Escherichia coli. G. Distribution of uropathogenic Escherichia coli by FISH in the same area showed in A with DAPI (left panel), specific probes (middle panel) and merge (right panel). Grey and white arrows indicate immune cells and tumor cells, respectively, at 40× magnitude. H. Transmission electron microscopy illustrating bacilli (arrow) morphologically compatible with Enterobacteriaceae in urothelial tumor cells from P #08, left panel and P #18, right panel. Triangle indicates nucleus. I. Spearman correlation between antibody-secreting cells and neutrophils in tumoral CD45+ cells analyzed by mass cytometry. Each dot represents one tumor and color code indicates the progression-free survival (PFS) status at 24 months.

FIG. 7. Escherichia coli-specific humoral and cellular TFH memory responses are associated with PFS. A. Experimental setting of the CD4+ T cell recall responses against bacteria (refer to M&M). B-C. Quantification of CXCL13 (B) and IFNγ (C) secretion levels in co-cultures of CD4+ T cells isolated before treatment (left panel) or post-treatment (right panel) and autologous monocyte-derived dendritic cells (DC) loaded with bacterial lysates for 2 days. Patients with relapse and/or death before 24 months (gold) and without (purple) are shown. Statistical analyses used unpaired t-test (Mann-Whitney U-test). D-E. Cohort 1 (PANDORE). Kaplan-Meier curves and Cox regression analysis of PFS of MIBC patients treated by pembrolizumab according to IgG against BCG (D) and Escherichia coli (E) using titer medians as cut-off values at baseline. Statistical analysis used Log-rank tests. F-G. NABUCCO and MATCH-R validation cohorts. Percentages of pathological complete response (pCR) in response to neoadjuvant ipilimumab and nivolumab in MIBC patients from NABUCCO trial according to IgG titers against E. coli using titer medians as cut-off values at baseline (F). Progression free survival Kaplan-Meier curves and Cox regression analysis in metastatic MIBC patients treated with pembrolizumab according to E. coli-specific IgG titers using medians as cut-off values at baseline (G). Statistical analyses used Chi-square test (F) and Log-rank test (G).

FIG. 8. Consort diagram and follow up. A. PANDORE study flow chart and study populations. B. Study design and collection of biological samples during PANDORE clinical trial. Orange, grey and green arrows correspond to collection of fixed tumors (FFPE tissues), blood and fresh+fixed tumors, respectively. C. Swimmer plot showing the follow-up of the population in whom safety was assessed (n=39 patients).

FIG. 9. CD8+ T cells and their vicinity with PDL-1+ cells post-pembrolizumab predict PFS. A. Tumor mutational burden (TMB) in TURBT samples according to the progression-free survival (PFS) endpoint (PFS of <versus ≥24 months, left panel) and Kaplan-Meier curve (right panel) and Cox regression analysis of PFS. Statistical analyses used unpaired t-test (Mann-Whitney U-test) and Log-rank test. B. Stacked bar chart showing the percentages of patients with pathological response according to combined positive score (CPS) (left panel). Clone 22C3, cut-off 10%. Kaplan-Meier curves and Cox regression analysis of PFS (middle panel) or overall survival (right panel) of MIBC patients treated by pembrolizumab according to CPS with 10 cutoff at baseline. Statistical analyses used Log-rank test. C-D. Kaplan-Meier curves and Cox regression analysis of PFS according to CD8+ TILs (left panel) and proximity of PD-L1+ with CD8+(right panel) using medians as cut-off values, at baseline (C) and after treatment (D). Statistical analyses used Log-rank test. E-F. Fold ratios in log for between mean values of each PD-1+ T cell subset (E) or B cells (F) between non progressors versus progressors (upper panels) and P-values of log-rank tests (lower panels) at baseline (F) or after 3 cycles of pembrolizumab (E, also refer to FIG. 2D-E)) assessing the contribution of each CD4+T (E) or B cell (F) subset to PFS. CD4+ T cell subsets were evaluated using mass cytometry (n=28 samples), clustering method (PhenoGraph) followed by manual annotation and manual gating. B cells subsets were evaluated using mass cytometry (n=31 samples) followed by manual gating). Statistical analyses used Log-rank test.

FIG. 10. Cluster 9 and 13 within circulating CD4+PD1+ TCM or TEM T cells predicted PFS in PANDORE. Kaplan-Meier curves and Cox regression analysis of PFS of MIBC patients treated by pembrolizumab according to cluster 9 before (upper panel) and cluster 13 after (bottom panel) 3 cycles of pembrolizumab using medians as cut-off values. Statistical analyses used Log-rank test.

FIG. 11. Tumoral TFH-like cells induced by neoadjuvant pembrolizumab predict PFS in MIBC patients. A. Distance between tertiary lymphoid structures (TLS)-like and TFH-like, defined as CD4+CD38+PD-1+ by immunostaining on TURBT and cystectomy samples. B. Fold ratio in log between mean values of patients with progression-free survival (PFS) < or ≥24 months (top panel) and P-values of log-rank tests (bottom panel) after 3 cycles of pembrolizumab (right panel) assessing the contribution of each tumoral mononuclear phagocytes (MNP), granulocytes, B and TIL cell subset, using medians for cut off values, to PFS. Immune cells were evaluated using mass cytometry (n=11 samples) and manual gating. Statistical analyses used Log-rank test. C. Kaplan-Meier curves and Cox regression analysis of PFS according to tumoral CD38highCD28+ non-Treg in CD4+ TILs using median for cut-off values. Statistical analysis used Log-rank test. D. Spearman correlation between B cells and CD38highCD28+CD4+ T cells in intra-tumoral CD45+ cells analyzed by mass cytometry, each dot representing one patient.

FIG. 12. CCL19 serum levels increased in responders during pembrolizumab. A. Single cell transcriptomics of CD45+. Frequencies of cluster 2 and cluster 6 among CD4+ according to sample origin. B Longitudinal changes of plasma CXCL9 (left panel) and CCL19 (right panel) before versus after 1st injection of pembrolizumab according to progression at 24 months (progression: gold, relapse-free: purple). Each line represents one patient. The dotted line shows the lower threshold of detection. Statistical analyses used paired t-test (Mann-Whitney U-test). C. Kaplan-Meier curves and Cox regression analysis of progression-free survival (PFS) of PANDORE patients according to the medians of CCL19 fold ratios between levels monitored before versus after 1st pembrolizumab. Statistical analysis used Log-rank test.

FIG. 13. MIBC containing CXCL13 producing TFH-like cells responded to ex vivo exposure to pembrolizumab. Percentages of CD38highCD28+PD-1high (cluster 28) in CD4+ TILs (left panel) and Spearman correlation between cluster 28 and CXCL13 production at D3 of in sitro assay in the control condition. Box plots display a group of numerical data through their 3rd and 1st quartiles (box), mean (central band), minimum and maximum (whiskers). Each dot represents one tumor. Statistical analyses used unpaired t-test (Mann-Whitney U-test).

FIG. 14. Phenograph of CD8+ TILs in MIBC containing TFH-like cells. A. Spearman correlations between clusters of CD8+ TILs and cluster 21 of CD4+ TILs (refer to FIG. 13A). B. Concentrations of CCL2, CXCL10, CXCL13, IL-4, IL-5 and TNFα measured in the Day 3 in sitro assay using anti-CD38 mAbs in addition to anti-PD-1 mAbs. Each dot and line (for paired specimen) represent one tumor (mean of three replicate wells). Statistical analyses used unpaired t-test (Mann-Whitney U-test).

FIG. 15. IgG4 labeling to follow pembrolizumab targets in blood and bladder tumors. A-C. Percentages of IgG4+ cells (supposedly binding pembrolizumab) within peripheral PD-1+CD4 metaclusters defined by mass cytometry, unsupervised clustering (PhenoGraph) and manual gating. For each subset, the mean percentage between three values determined before the 2nd, the 3rd injection and after 3rd injection, for each patient is indicated (A) while the detailed results for each subset in the longitudinal follow up is shown in B and an example illustrating TFH and TFH-like TCM metacluster (cluster 6, 9, 18 and 20) (bottom) and TEM (cluster 13) (top) is depicted in C. Each line represents one patient. Statistical analyses used paired t-test (Mann-Whitney U-test). D. Ratios of the IgG4 mean fluorescence intensity (MFI) between in the CD38high or CD38− populations assessed by flow cytometry after 3 days of in sitro stimulation with anti-PD-1 mAbs in MIBC analyzed in sitro subdivided according to their immunoreactivity (refer to FIG. 4) (upper panel). Statistical analyses used unpaired t-test (Mann-Whitney U-test). Spearman correlation between the MFI of IgG4 expression obtained post-PD-1 blockade in sitro and the baseline MFI of PD-1 in the two subsets of CD4+(CD38hi versus CD38−).

FIG. 16. Humoral and cellular immune responses against urinary commensals in MIBC patients. A. Proportions of patients with positive or negative LPS staining and presence or absence of neutrophils in TURBT samples (n=33 samples). B. Stacked bar chart showing the number of culturomics leading to the mass spectrometry identification of commensals in tumor specimen or their control (transport liquid) and surrounding tissues (non-tumor sample and their respective transport liquid) (n=4 patients with paired samples). C. Paired (before and after neoadjuvant pembrolizumab) recall responses directed against Escherichia coli, Streptococcus sanguinis and Staphylococcus capitis are depicted. CD4+ T cells incubated with Dynabeads® and CD4+ T cells co-cultured with and without monocyte-derived dendritic cells were used as positive and negative controls, respectively. Each line and each dot represent one patient and one sample, respectively. Statistical analyses used paired t-test (Mann-Whitney U-test). D. Flow cytometric relative quantifications of specific serum IgA and IgG titers directed against a variety of bacteria commonly found in urinary or digestive tracts or skin layers as well as BCG. Two dots per patient are shown (titer before and titer after pembrolizumab) for each PANDORE patient. E. Longitudinal follow up of IgG against BCG in serum of PANDORE patients by flow cytometry. Each line and each dot represent one patient and one sample, respectively. Statistical analyses used paired t-test (Mann-Whitney U-test). F-G. Kaplan-Meier curves and Cox regression analysis of progression-free survival (PFS) of PANDORE according to total IgG (F) and IgG against S. sanguinis (G) considering medians at baseline as cutoff values. Statistical analysis used Log-rank test.

FIG. 17. Flow chart of the samples and techniques used for the translational research in the PANDORE clinical trial

FIG. 18. Whole blood stimulation with pasteurized E. coli Q1696 in two prototypic individuals, one after a E coli urinary infection (A), the other one at distance from any diagnosis of urinary infection (B). Whole heparinized blood was incubated with decreasing dosing of pasteurized E. coli Q1696 (from 330 up to 3300 cfu/10 ul/well)+/− neutralizing anti-MHC class I or II antibodies for 24 hrs. Supernatants were obtained after centrifugation of the coculture and monitored in a multiplex 42 ELISA assay for chemokines and cytokines. Only IFNg, CXCL13, and CXCL9 showed a dose response and a blockade with anti HLA antibodies (both MHC class I and II) for A only.

FIG. 19. Tumor infiltrated lymphocytes contain memory follicular T helper cells secreting CXCL13 upon ex vivo restimulation with E. coli+/−anti-PD1 Ab. Overview of the in situ/in vitro (in sitro) test with heat killed bacteria (top panel). Multiplex immunoassays monitoring of CXCL13 (middle panel) and CCL19 (bottom panel) TLS-specific chemokines in in sitro stimulation of two to 5 freshly dissociated muscle invasive bladder tumors (MIBC) with heat inactivated E. coli. Different culture conditions are annotated as well as the neutralization of MHC class II complexes with specific antibodies. Medians of duplicate-triplicate wells are shown for CXCL13 (each dot corresponds to one MIBC sample) and two independent experiments performed in duplicate-triplicate wells are shown for CCL19 (each dot corresponds to one well). Statistical analyses used paired t test (middle panel) and Two-way ANOVA (bottom panel).

FIG. 20. Profile of immunoglobulin isotypes and frequencies of antibody-secreting cells (ASC) within freshly dissociated MIBC after co-culture with bacteria.

A-B. Concentrations of IgG subclasses, IgA, IgM and IgE measured by multiplex immunoassays right after dissociation and digestion of 3 MIBC at diagnosis (A, left panel) and in the supernatants (A, right panel) and percentages of ASC in CD45⁺ TILs evaluated by flow cytometry, after 3 days of co-culture with or without restimulation with heat-killed bacteria in in sitro assays.

FIG. 21. E. coli-specific B cells located in fresh tumor sample react and bind to E. coli pks positive.

The CD45+ part of fresh tumor sample was cell sorted and then, expanded in IL-4+IL21+anti-CD40 Ab for 5-6 days twice in a raw. Then, the expanded tumor infiltrating B+/−T cells were exposed to fluorescent dead E. coli (either E. coli pks+ or E. coli pks−) for 4 hours and flow cytometry analysis gating on CD27+CD38+/−CD19+CD3− cells was performed. A typical Facs dot plot is displayed, showing not only E coli-binding B cell subsets but also CD8 and myeloid T cells. In this set of experiment, another isolate of E. coli pks+ or pks− isolated from a colorectal cancer (CRC) Lynch patient (for the pks−) or from a microsatellite stable CRC patient (for pks+) were used. The two strains could bind to different subsets of tumor infiltrating B cells.

FIG. 22. A. Seroreactivities monitored against E. coli in different patient subsets and prognosis in kidney cancers. Flow cytometric analyses of mean fluorescence intensity of patient serum compared with a standardized batch of serum polyclonal IgG, monitored in metastatic kidney or lung or bladder (MIBC) patients or hematological malignancies or a single or a pool of health volunteers (HV). B. Cox regression analysis and Kaplan Meier overall survival curves for 16 metastatic kidney cancer patients (p value indicated comparing patients with a staining index superior to the median of the cohort). C. Longitudinal and paired follow up of serum titers over time (baseline and after 2 months of pembrolizumab).

FIG. 23. Head-on comparisons between the long and tedious DC/T/bacterium assay (A) and the short whole blood-based assay (B) to monitor recall responses against pasteurized E. coli or other bacteria. Each bacterium is exemplified in the X axis. K. pneumoniae=Klebsiella pneumoniae, E. coli=Escherichia coli, F. nucleatum=Fusobacterium nucleatum, Pool1 is a combination of the three latter ones. The Enterocloster genus (E. clostridioformis, bolteae, hathawayi=pool 2) are poorly immunogenic. Here, we show the concentrations of IFNg release over 48 hrs (A) or 24 hrs (B).

FIG. 24. Whole blood reactivity to various sets of bacteria using the rapid T cell assay. Each bacterium has been described in Goubet et al. (Cancer Discovery, 2022). For this rapid T cell assay in 96 well plate, 200 μl of whole blood, 100 μl of mitogen (CD49/CD28), 100 μl of pasteurized bacteria (15 000 cfu) or bacterial pool of 2-3 bacteria (10 000 cfu for each) have been distributed and incubated for 24 hours at 37° C. CXCL13, IFNg and IL-10 have been monitored by ELISA assay. The figure shows the cytokine concentrations of IFNg and II-10 for 2 individual cancer patients with MIBC, in triplicate, determined using commercial ELISA.

FIG. 25. Blood T cell reactivities against E. coli long peptide pools. Same procedure as the one described for whole pasteurized E. coli, but incubated 10 ug/ml of each peptide pool. MOG is a negative control in HV (myeline basic protein). Each bar is the concentration of IFNg or IL-10 by ELISA in pg/ml well by well in triplicate wells (w/w).

### DETAILED DESCRIPTION

Presently, intravesical BCG instillations are used in the treatment of superficial bladder cancers, but not in infiltrating muscle-invasive bladder cancer (MIBC) indications. The inventors however confirmed the presence of live bacilli within bladder tissues of MIBC patients and showed that such intravesical BCG instillations could be useful for treating MIBC, especially in combination with sequential ICBs.

They also showed the presence of Escherichia coli (E. coli) in tumor and myeloid cells of MIBC patients in PCR and electron microscopy and could cultivate Staphylococcus capitis in bladder cancers. In addition, they identified a strong correlation between neutrophils (expected to be attracted by bacterial infection) and ASC within tumor infiltrating CD45⁺ cells after treatment with an anti-PD1 antibody, suggesting that innate and humoral immune responses against bacteria might cooperate in the tumor beds. In addition, they identified that CXCL13-producing T_{FH} responses against both E. coli and S. capitis were associated with the absence of progression at 24 months, suggesting that S. capitis can also be useful in the treatment of MIBC.

According to a first aspect, the present invention thus pertains to an immunogenic composition comprising antigens from bacteria selected from the group consisting of Escherichia coli (E. coli), Staphylococcus capitis (S. capitis), Bacillus Calmette-Guérin (BCG) and mixtures thereof, for use in treating muscle-invasive bladder cancer (MIBC).

According to a preferred embodiment, the immunogenic composition comprises antigens from uropathogenic E. coli (UPEC).

According to a particular embodiment, the immunogenic composition comprises E. coli and/or S. capitis and/or BCG bacteria (pasteurized, attenuated or alive), or fragments thereof. The skilled person will preferably select highly immunogenic strain(s) to use in said composition. Such a composition can be administered, for example, via intravesical instillation or via oral administration (in gastroresistant capsules, preferably containing a high amount of bacteria, for example at least 10⁹ or 10¹⁰ bacteria). Such a composition can be, for example, used to administer 10¹⁰ bacteria per day, every other week, for three weeks before each injection of anti-PD1/PDL-1 or CTLA4 Abs.

According to another particular embodiment, the immunogenic composition comprises E. coli and/or S. capitis proteins (e.g. virulence factors, fimbriae, iron acquisition gene products and/or glycoproteins, for example), or peptides from said proteins. In this embodiment, the proteins or peptides are administered in a formulation appropriate for vaccination in humans. By “formulation appropriate for vaccination in humans” is herein meant that the proteins are in a medium/buffer/solution which is pharmaceutically acceptable. Such a formulation can also comprise appropriate adjuvant (e.g., Freund adjuvant, Montanide or any other adjuvant known by the skilled person) and/or immunstimulant(s) (e.g., Poly(I:C) and CpG-ODN).

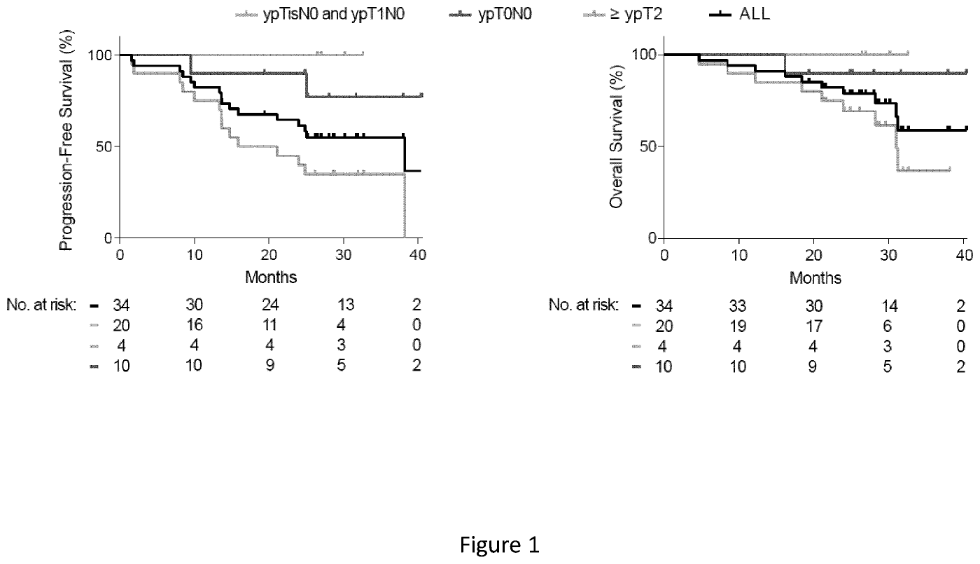

Hasanzadeh et al. described a multiepitope vaccine candidate against UPEC, which is a fusion of 3 epitope-rich domains of lutA and FimH antigens selected using bioinformatics approaches to predict the best B and T-cell epitopes of UPEC virulence proteins (Hasanzadeh et al., 2020). This construct can be used in the frame of the present invention. Alternatively, shorter peptides can be included in the immunogenic composition according to the invention. Examples of E. coli peptides which can be used when performing the invention are peptides comprising one or more of the epitopes described in FIG. 1 of the article by Hasanzadeh et al. (supra).

According to yet another particular embodiment, said composition comprises a nucleic acid (e.g., RNA or DNA) encoding E. coli and/or S. capitis proteins. The skilled person can for example use the mRNA vaccine technology recently used for Sars-Cov2 vaccines, or a DNA vaccine.

Protein or peptide vaccines, as well as nucleic acid vaccines are well known by the skilled person and can be administered by any appropriate route of administration, such as transcutaneous, intramuscular, nasal and/or lung spray, etc. According to a particular embodiment, the immunogenic composition is administered intramuscularly.

The immunogenic composition according to the invention is particularly useful when used in combination with an anti-PD1/PD-L1/PD-L2 Ab-based therapy. The phrase “anti-PD1/PD-L1/PD-L2 Ab-based therapy” herein designates any drug that antagonizes PD1 or PD-L1 or PD-L2. Although the currently used drugs antagonizing PD1 or PD-L1 or PD-L2 are monoclonal antibodies, other molecules specifically binding to PD1, PD-L1 or PD-L2 could be used for the development of future immune checkpoint blockers (ICB) such as, for example, antibody fragments or specifically designed aptamers. Of course, the phrase “anti-PD1/PD-L1/PD-L2 Ab-based therapy” encompasses any therapy with active molecules that antagonize PD1 or PD-L1 or PD-L2.

A combined treatment with (i) an immunogenic composition as above-described and (ii) an anti-PD1/PD-L1/PD-L2 Ab-based therapy (e.g., an anti-PD1 monoclonal antibody) is thus also part of the present invention. The protocol will vary depending on the formulation of the immunogenic composition. In such a treatment, the immunogenic composition should precede the ICB therapy. For instance, intramuscular administration of E. coli peptides or RNA should be performed twice, three to four weeks apart and then, 3 weeks later, the ICB should be administered. In case of oral uptake of encapsulated bacteria, the oral administration should precede by 3 days the ICB start, and should be taken 3 days in a raw, 3 days before each cycle of ICBs. The intravesical instillation of dead bacteria should be done the day before ICB at each cycle.

Another aspect of the present invention is the use of an antibody targeting an antigen from E. coli or from S. capitis, preferably from E. coli, more preferably from UPEC, in the treatment of muscle-invasive bladder cancer (MIBC). Such an antibody can be obtained by a method as described in Example 7 below.

By “antibody” is herein meant a polyclonal or monoclonal antibody of any isotype, as well as antibody fragments and engineered molecules derived from natural antibodies, such as chimeric antibodies, humanized antibodies, single chain antibodies, bispecific antibodies, diabodies, scFv, Fab, F(ab)₂, and di-, oligo- or multimers thereof.

According to a particular embodiment, the antibody is conjugated or covalently linked to a cytotoxic drug. Antibody-drug conjugates (ADC) are a new class of highly potent biopharmaceutical drug composed of an antibody linked, via a chemical linker, to a biologically active drug or cytotoxic compound. These targeted agents combine the unique and very sensitive targeting capabilities of antibodies with the cell-killing ability of cytotoxic drugs.

To date, at least twelve ADCs have received market approval, including: ado-trastuzumab emtansine (Kadcyla™), brentuximab vedotin (Adcetris™), inotuzumab ozogamicin (Besponsa™), gemtuzumab ozogamicin (Mylotarg™), Moxetumomab pasudotox (Lumoxiti™), polatuzumab vedotin-piiq (Polivy™), Enfortumab vedotin (Padcev™), Sacituzumab govitecan (Trodelvy), Trastuzumab deruxtecan (Enhertu™) belantamab mafodotin-blmf (Blenrep™), loncastuximab tesirine-lpyl (ZYNLONTA™), and tisotumab vedotin-tftv (Tivdak), and nearly 100 investigational ADCs are currently in preclinical and clinical trials.

Highly cytotoxic small molecule drugs that are unsuitable for systemic administration alone can be used in ADC. Non-limiting examples of drugs which can be conjugated to anti-E. coli or anti-S. capitis antibodies in ADC according to the invention include payloads such as the topoisomerase I inhibitor deruxtecan, ozogamicin, emtansine, vedotin and mafodotin.

The antibodies and ADC according to the invention can be administered intravenously or via intravesical instillation.

When investigating the PANDORE single arm phase 2 trial (NCT03212651), which enrolled patients with MIBC treated with neoadjuvant pembrolizumab, the inventors obtained evidence that follicular helper T cells, a specialized subset of CD4⁺ effector T cells involved in germinal center reactions and affinity maturation of B cells, represent the main therapeutic target of neoadjuvant pembrolizumab, both in blood and bladder tissues. Baseline blood memory follicular helper CD4⁺ T lymphocytes (T_{FH}), together with B cells, became activated by pembrolizumab, facilitating the orchestration of tertiary lymphoid structures (TLS) and CD8⁺ TIL accumulation, translating into clinical benefit. Importantly, antibodies and CXCL13 producing CD4⁺ T cell memory responses against Escherichia coli-specific emerged as robust biomarkers of clinical benefit. The inventors also observed that preexisting responses against Staphylococcus capitis and BCG indicate that the patient is likely to respond to an immune-oncology treatment.

The inventors also showed the predictive value (for the response to immunotherapy such as PD-1 blockade) of the E. coli-specific IgG titers to kidney cancers from the NIVOREN cohort (Example 10).

According to another of its aspects, the present invention thus relates to in vitro methods of determining if an individual having a muscle-invasive bladder cancer (MIBC) or a kidney cancer is likely to respond to an anti-PD1/PD-L1/PD-L2 Ab-based therapy, comprising a step of assessing, in a sample from said patient:
(i) the level of E. coli-specific IgG,
(ii) the level of S. capitis-specific IgG and IgA,
(iii) the level of BCG-specific IgG, and/or
(iv) the level of E. coli-specific CXCL13 producing follicular helper CD4⁺ T (T_{FH}),
   wherein if said level(s) is(are) superior to (a) predetermined threshold(s), the individual is likely to respond to said anti-PD1/PD-L1/PD-L2 Ab-based therapy.

According to a preferred embodiment the level of E. coli-specific IgG is measured.

When performing the above method, the skilled person can chose any appropriate method to measure the levels of E. coli-specific IgG, S. capitis-specific IgG and IgA, BCG-specific IgG and/or whole blood reactivity to E. coli. For example, the skilled person can use a bacterial flow cytometry assay as described in the experimental part below (§ 5.3 of the Materials & Methods), or an ELISA assay as described by Hasanzadeh et al. (2020). The skilled person can easily develop another immune assay, such as an ELISA assay, to measure the level of any of the reciped IgG. Then, from a cohort of patients with MIBC or kidney cancer treated with an anti-PD1/PD-L1/PD-L2 Ab-based therapy, the skilled person can determine, by routine experimentation, an appropriate threshold cognate to the technique used for measuring the IgG level.

Several assays can also be used for assessing the level of E. coli-specific CXCL13 producing T_{FH} when performing the above method.

According to a particular embodiment, the level of E. coli-specific T_{FH} is assessed by a method comprising:
(i) contacting peripheral blood cells (heparinized whole blood) from the individual with pasteurized E. coli, or with immunogenic peptides from an E. coli amino acid sequence, in appropriate conditions to stimulate said cells, and
(ii) following cell stimulation (typically, after 22-24 hrs), measuring the expression of CXCL13 in the supernatant, and/or
   wherein if the level of CXCL13 expression is elevated, (i.e., superior to the negative control), the individual is likely to respond to the anti-PD1/PD-L1/PD-L2 Ab-based therapy.

Follicular T helper cells (T_{FH}) are typically secreting CXCL13 and IL-21 upon activation. Then, T_{FH} play a helper role to stimulate MHC class I-restricted CD8+ effector cells, the latter being capable of releasing Tc1 cytokines (typically IFNg and CXCL9). Therefore, E. coli-specific T_{FH} will activate bystander or antigen specific cytotoxic CD8⁺ T cells to produce IFNg and CXCL9. The inventors found that individuals who have a memory T_{FH} response directed against E coli also harbored CD8⁺ T cells releasing CXCL9 and IFNg following restimulation with E coli, in a dose dependent manner.

Hence, according to another particular embodiment, the level of E. coli-specific T_{FH} is (indirectly) assessed by a method comprising:
(i) contacting peripheral blood cells (heparinized whole blood) from the individual with pasteurized E. coli, or with immunogenic peptides from an E. coli amino acid sequence, in appropriate conditions to stimulate said cells, and
(ii) following cell stimulation (typically, after 22-24 hrs), measuring the expression of IFNg, CXCL9 and/or TNFa in the supernatant,
   wherein if the level of IFNg, CXCL9 and/or TNFa expression is elevated, (i.e., superior to the negative control), the individual is likely to respond to the anti-PD1/PD-L1/PD-L2 Ab-based therapy.

Examples of peptides that can be used for performing the above methods include, but are not limited to 15-mer overlapping peptides from an E. coli amino acid sequence, such as those described in Table 8 or in FIG. 1 of Hasanzadeh et al., as well as peptides (of at least 9 amino acids, preferably of 15 amino acids or more), overlapping or not, covering at least part of at least one E. coli antigen (preferably one or several E. coli-specific antigens), as illustrated in Example 10, and FIG. 25. Peptides from several strains of E. coli can also be pooled.

In the above methods, the expression level of a cytokine/chemokine can typically be considered as elevated if it is at least 1.5 times above the negative control (unstimulated by bacteria), preferably with a dose-dependent effect of cytokine/chemokine release.

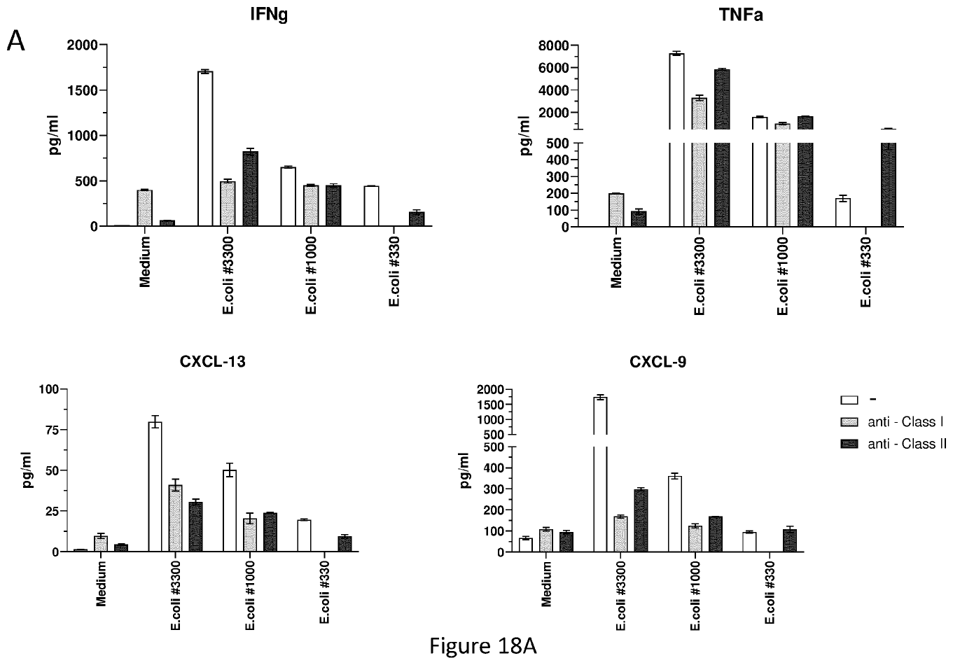

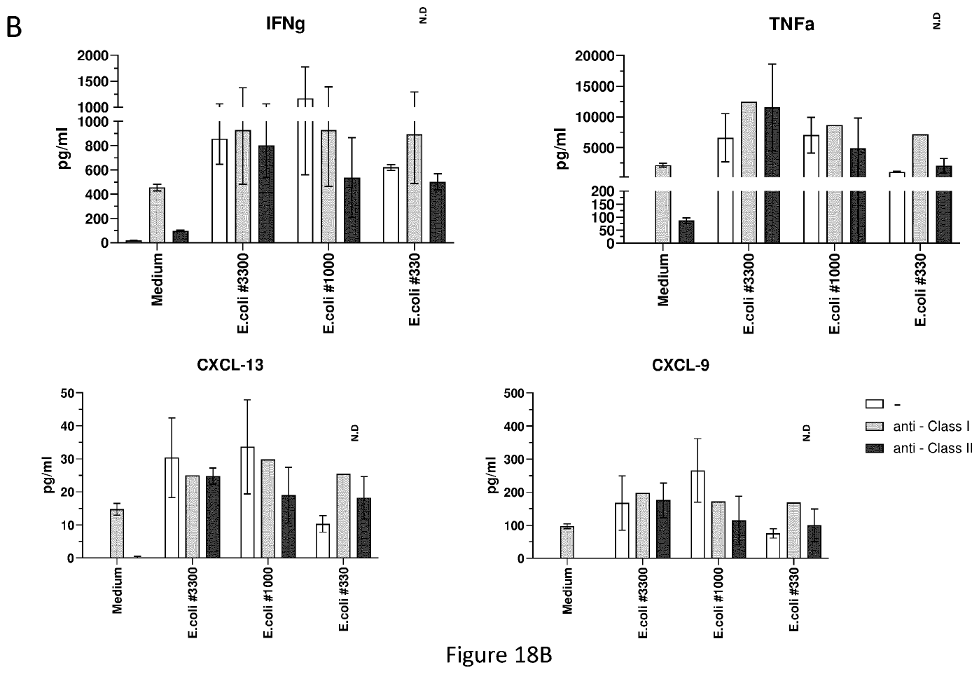

A more detailed protocole of the above methods is described in the experimental part (§ 5.4 of the Materials & Methods, Example 8, FIG. 18).

Optionally, anti-MHC class I or II antibodies (anti-HLA-ABC or DP-DQ-DR respectively) can be added at 10 ug/ml to the solution containing pasteurized bacteria to assess the specificity of cell-mediated immunity.

According to another particular embodiment, the level of E. coli-specific T_{FH} is assessed by a method comprising:
(i) generating monocyte-derived dendritic cells (mo-DC) from PBMC from the individual;
(ii) incubating said mo-DC with a suspension of Escherichia coli (to allow processing of the antigenic load and cross-presentation into MHC class I and II molecules);
(iii) adding an antibiotic (to avoid culture contamination and DC and lymphocyte death);
(iv) co-culturing said mo-DC with memory CD4⁺ T cells from the individual during 1 to 3 days, preferably 2 days; and
(v) measuring the expression of CXCL13 and/or IL-21 (prototypic T_{FH} chemokine and cytokine) in the supernatant,
   wherein if the level of CXCL13 and/or the level of IL-21 expression is elevated, the individual is likely to respond to the anti-PD1/PD-L1/PD-L2 Ab-based therapy.

In the above method, the expression level of a cytokine/chemokine can typically be considered as elevated if it is at least 1.5 times above the negative control (unstimulated by bacteria), preferably with a dose-dependent effect of cytokine/chemokine release.

In the above methods, the skilled person can also adapt the threshold to the protocole used for measuring the level of E. coli-specific T_{FH}, by routine experimentation on a cohort of MIBC patients.

Another aspect of the present invention is an in vitro method for determining if an individual having a muscle-invasive bladder cancer (MIBC) responds to an anti-PD1/PD-L1/PD-L2 Ab-based therapy, comprising:
(i) comparing the serum level of CXCL13 in said patient before and after administration of said anti-PD1/PD-L1/PD-L2 Ab-based therapy, and/or
(ii) comparing the serum level of CCL19 in said patient before and after administration of said anti-PD1/PD-L1/PD-L2 Ab-based therapy,
   wherein an increase of CXCL13 and/or CCL19 three weeks after the first administration of said anti-PD1/PD-L1/PD-L2 Ab-based therapy indicates that the individual responds to the therapy.

The present invention also relates to an ex vivo method for determining if an individual having a muscle-invasive bladder cancer (MIBC) in partial remission after a treatment with an anti-PD1 IgG4 responds to said treatment, comprising immunostaining samples corresponding to representative slides of TURBT and cystectomies with antibodies directed against IgG4, PD1, CD4 and CD38, wherein if co-stained cells are observed, the individual responds to the treatment, and if no co-stained cells are observed, the treatment can be stopped.

### EXAMPLES

### Materials and Methods

### 1. Clinical Studies

### 1.1. Study Design and Population

PANDORE. PANDORE is a prospective, single-arm phase 2 trial testing the antitumor activity of preoperative pembrolizumab monotherapy in patients with histologically confirmed (T2-T4aN0M0) transitional cell carcinoma of the bladder (ClinicalTrials.gov: NCT03212651). Pembrolizumab was given at the recommended dose of 200 mg every three weeks for three cycles followed by cystectomy with appropriate lymph node dissection.

Patients were 18 years of age or older, ineligible for cisplatin or refused cisplatin-based chemotherapy, had diagnostic transurethral resection (TURBT) blocks available and had adequate hematologic and end-organ function. Key exclusion criteria included documented severe autoimmune disease, chronic infectious disease, use of systemic immunosuppressive medications and prior use of immune checkpoint inhibitors. Surgery was scheduled 1 to 3 weeks after the last pembrolizumab infusion. Baseline work-up included thorax/abdomen/pelvis computed tomography (CT) and standard blood analyses (hematology, biochemistry and coagulation parameters). Neither PET-CT nor MRI was required for baseline imaging. There was no dose modification of pembrolizumab. Patients with grade 2-4 pembrolizumab-related toxicity (except for grade 2 infusion-reaction) were required to permanently discontinue pembrolizumab administration and to proceed with radical cystectomy.

Thirteen patients (38.2%) had post-operative complications (grade 3-4) such as infection (n=3), metabolism and nutrition disorders (n=1), procedural complications (n=2), renal and urinary disorders (n=4), gastrointestinal disorders (n=1) and musculoskeletal and connective tissue disorders (n=1).

The trial was conducted by the French Genitourinary Group (GETUG) and funded by MSD which provided the drug. This study was approved by the ethics committee CPP Est-III in December 2017, the French National Agency for the Safety of Medicines and Health Products (ANSM) in November 2017 and was conducted in accordance with the protocols and Good Clinical Practice Guidelines defined by the International Conference on Harmonization and the principles of the Declaration of Helsinki.

NABUCCO (validation cohort). NABUCCO study enrolled 24 patients with stage III UC treated with ipilimumab (cycle 1), ipilimumab and nivolumab (cycle 2) and nivolumab (cycle 3) followed by resection (van Dijk N et al, 2020). A baseline serum sample were available for 23 patients who defined the validation cohort 2.

MATCH-R (validation cohort). MATCH-R is a prospective trial studying the evolution of clonal architecture of tumors from patients with advance cancer and treated with molecular targeted agents to identify mechanisms of acquired resistance (ClinicalTrials.gov: NCT02517892). The design of the study has been reported previously (Recondo et al, 2020). Briefly, the primary objective of this study is to characterize molecular mechanisms of resistance to targeted therapies and immunotherapy by NGS and the development of patient derived xenografts (PDX) and cell lines.

Patients treated with pembrolizumab with metastatic muscle invasive bladder cancer (MIBC) were enrolled in our study and define the validation cohort 3. Baseline, on-treatment and post progression biopsies are performed in patients treated with pembrolizumab. In addition, blood samples are collected longitudinally throughout the treatment and at progression in patients for collecting serum, plasma and circulating tumour DNA (ctDNA).

Untreated bladder cancers. A cohort of 18 patients with untreated non-metastatic MIBC was included in this study. Surgeries were performed in the University Paris-Saclay Hôpital Foch between August 2017 and July 2021, and tumor samples were collected with appropriate written informed consent for use of clinical data and scientific purposes according with protocol reviewed and approved by institutional ethic committee (No IRB 00012437). Before radical cystectomy, all patients were treated with cefazolin. In case of urinary tract infection, additional antibiotics have been used according to the sensitivity to other antibiotics.

### 1.2. Study Endpoints and Statistics

Outcomes. The primary endpoint was the pathological complete response (pCR) defined as the absence of cancer cells in the bladder and the absence of microscopic lymph node metastases on the final cystectomy specimen (ypT0N0). Histopathological examination was performed locally in each participating center by experts GU pathologists on the resected primary tumor and lymph node specimens. Pathological staging was done according to international standards and protocols. Secondary endpoints were: (i) the pathological response (pR) defined as the absence of muscle invasive carcinoma (<ypT2 disease) and the absence of microscopic lymph node metastases (ypN0) on the final cystectomy specimen (defined as major pathological response, MPR), (ii) the number of participants with a Grade 3-4 adverse event according to NCI CTCAE version 4.0, related or not to pembrolizumab (Non-related adverse events and immune related adverse events were graded according to NCI CTCAE version 4.0 and reported throughout the study), (iii) the number of patients having cystectomy. Survival outcomes were progression-free survival (PFS) defined as the time from the first dose of pembrolizumab to progression (local relapse or metastases, death) whichever occurs first and overall survival (OS) defined as the time from the first dose of pembrolizumab to death whatever the cause.

Statistical considerations. The sample size was calculated on a precision analysis rather than power analysis of the main outcome. The sample size was based on the confidence interval (CI) and the CI half-width (the CI half-width is the margin of error associated with the confidence interval). For a proportion of pathological complete response rate equal to 30% and a 95% confidence interval, 36 patients were to be included in order to obtain an estimate with certain precision of 0.15. Considering that a 10% rate of patients who did not undergo cystectomy, 40 patients were needed to be recruited.

Response rates were calculated with their 95% CI by using the exact Clopper-Pearson method (based on the exact binomial distribution). Progression Free Survival and Overall Survival were estimated by the Kaplan-Meier method. Median follow-up was estimated using reverse Kaplan-Meier method. The main analysis included patients who met the eligibility criteria, had at least one cycle of pembrolizumab and underwent cystectomy. Safety analysis included all patients who met the eligibility criteria and received at least one dose the study drug. The clinical data cut for analysis occurred in August 2021. Data were analyzed using SAS software v-9.4 (Cary, NC), GraphPad Prism version 7 for Windows, GraphPad Software (La Jolla California USA).

### 2. Sample Collection and Processing

### 2.1. Sample Collection

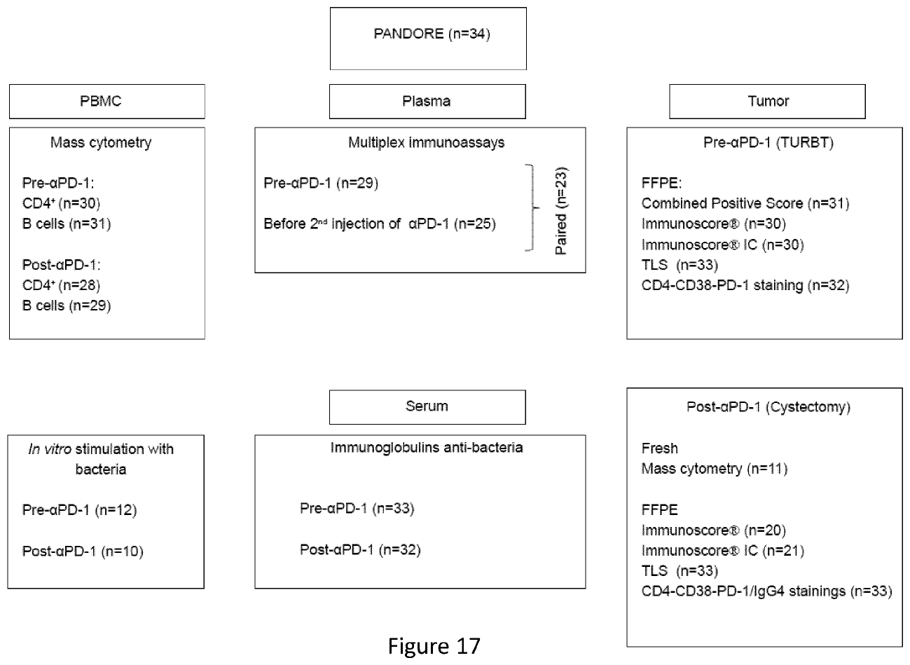

PANDORE. Peripheral blood samples from patients enrolled in PANDORE were drawn and collected into sterile vacutainer tube uncoated and tubes coated with heparin. Blood samples were collected at baseline (before 1st cycle of pembrolizumab), right before the 2nd cycle and the 3rd cycles of pembrolizumab, before and one month after surgery. Tumors before treatment were collected as Formalin-Fixed Paraffin-Embedded (FFPE) tissues. Tumor after treatment were collected freshly in PRMI and as FFPE tissues. Available and analysed samples are shown in FIG. 17.

MATCH-R (validation cohort). Peripheral blood samples from patients enrolled in MATCH-R were drawn and collected into sterile vacutainer tubes uncoated for serum collection.

Untreated bladder cancers. Fresh tumors were collected in PRMI at +4° C.

### 2.2. Tissue Digestion

Tumor pieces and non-involved bladder tissues were collected in RPMI 1640 (GIBCO, Life Technologies, ref: 31870-025), at +4° C. The samples were stored less than 17 hours at +4° C. before processing. Tissues were weighed and digested, enzymatically, and mechanically. Briefly, tissues were cut in small pieces using scalpels and forceps in a petri dish. The small pieces were then dissociated in a gentleMACS Octo Dissociator (Miltenyi, Germany) using the program ‘37C_h_TDK_1’ in a dissociation medium, which consisted of RPMI, Collagenase IV (50 IU/mL, Sigma-Aldrich, Cat #C2139), hyaluronidase (280 IU/mL, Sigma-Aldrich, Cat #H6254), and DNAse I (30 IU/mL, Sigma-Aldrich, ref: 260913). Dissociation time lasted 1 hour under mechanical rotation and heating. The samples were then through a 100 μm stainer, crushed and washed in NaCl (Versylene Fresenius Kabi, France) at 1500 rpm for 5 min. The pellets were resuspended in an adapted volume of NaCl 0.9%. If required, red blood cell lysis was performed using RBC lysis Buffer 1× (BioLegend Cat #420301). For untreated samples, an aliquot of 50 μL were used to quantify the total number of cells and the fraction of CD45+ cells with Precision Count Beads™ (Biolegend, Cat #424902) by flow cytometry following manufacturer's protocol. For PANDORE samples, the total number of cells were counted using a hemocytometer.

### 2.3. Serum Collection

Uncoated tubes were centrifuged for 10 min at 1800 rpm. Serum was aliquoted and stored at −80° C. until measurements.

### 2.4. Peripheral Blood Mononuclear Cell Isolation and Plasma Collection

Whole bloods were layered on a Lymphocyte Separation Media (VWR, Cat #25-072-CV) in Leucosep tubes (Dutscher, Cat #227288). Tubes were centrifuged for 15 min at 2000 rpm at room temperature. Plasma were collected and stored at −80° C. until measurements. Peripheral blood mononuclear cells were collected, washed in NaCl 0.9% and counted using hemocytometer. Cells were washed and resuspended in fetal calf serum containing 10% of dimethyl sulfoxide (DMSO, SIGMA, Cat #276855) for storage in liquid nitrogen.

### 3. Immunophenotyping

### 3.1. Mass Cytometry

Samples. PBMC: after thawing, cells were washed and resuspended in RPMI+10% FCS+DNAse (30 IU/mL). After 1 h of incubation at 37° C., cells were washed and counted using Vi-Cell XR Cell Viability Analyzer (Beckman Coulter). Two million of cells were used. Tumor: at least 1M of cells were used for staining by mass cytometry.

Sample preparation. Cells were stained for viability and proliferation with rhodium (Rh103, Fluidigm Cat #201103A) (1:100) and IdU (Fluidigm Cat #201127) (1:2000), respectively, in RPMI+10% FCS for 20 min at +37° C. Cells were washed in staining buffer (PBS 1×+BSA 0.2%+EDTA 2 mM). Cells were incubated during 5 min with Fc blocker (FcX, BioLegend Cat #422302) before being stained with extracellular antibodies. For PBMCS, cells from each timepoint were labelled with a unique barcode by incubating with CD45-antibodies conjugated to distinct metal isotopes (Cadnium 106, 110, 111, 112, 114) before pooling. Antibodies were either purchased pre-conjugated from Fluidigm or purchased purified and conjugated in-house using MaxPar X8 Antibody Labeling Kit or Maxpar MCP9 Antibody Labeling Kit (Fluidigm) according to the manufacturer's instructions. Cells were then stained with a PBMCs or tumor panel of antibodies (table 2) for 30 min at +4° C. For PBMCs, samples were washed, fixed and permeabilized (Foxp3/Transcription Fractor Staining Buffer Set eBiosciences Cat #00-55-23-00) for 40 min at +4° C. before being stained with intracellular antibodies (table 2) for 30 min at +4° C. All samples were washed, incubated in Fix and Perm Buffer (Maxpar Fix and Perm Buffer Fluidigm Cat #201067) for 5 min before being fixed in Iridium intercalator (Iridium191/193 Fluidigim Cat #201192B) (1:4000) diluted in PBS 1× containing 1.6% formaldehyde for 40 min at room temperature. Cells were washed and stored until acquisition at +4° C.

Data Acquisition. Cells were counted, washed and resuspended in Maxpar Cell Acquisition Solution at 0.5×106/mL and mixed with 10% EQ Beads immediately before acquisition on Helios mass cytometer using noise reduction, event length limits of 10-150 pushes. An average of 500,000 events were acquired per sample at a flow rate of 0.03 mL/min. Mass cytometry standard files were normalized to a global standard determined for each log of EQ beads using CyTOF Software v. 6.7.1014 (Fluidigm).

Analyses. Analysis was performed with FlowJo software (Tree Star). Non-events (doublets and debris etc.) were removed from the analysis using the Gaussian discrimination channels (Center, Offset, Width and Residual) as recommended by Fluidigm. Circulating CD4+ T cells were clustered within and across samples using PhenoGraph (Levine et al, 2015). One cluster representing less than 0.075% of the total CD4+ T cells (n=854/1159561 cells) was not considered in the following analysis. Clusters were then annotated manually and merged in metaclusters according to their relative expression of PD-1 (relative expression >0: PD-1+, relative expression=0: PD-1−) and effector-memory phenotype (Table 3). Briefly, naive, effector memory re-expressing CD45RA (TEMRA), effector T cells (TE), effector memory T cells (TEM) and central memory T cells (TCM) were defined as CD45RA+CD28+CD27+CD127+, CD45RA+CD28− and/or CD27−CD127low, CD45RA−/lowCD28−/lowCD27−/lowCD127−/low, CD45RA−/lowCD28+CD27+CD127−/low, CD45RA−/lowCD28+CD27+/lowCD127+, respectively. Then, manual gating was performed on metaclusters to define subsets (helper T cells). Tfh, Th1, Th2, Th17 and others CD4+ T cells were defined as CXCR5+, CXCR3+CXCR5, CXCR3−CXCR5−CCR4+, CXCR3−CXCR5−CCR4−CCR6+ and CXCR3−CXCR5−CCR4−CCR6−, respectively Manual gating was performed for the analysis of B cells in PBMCs. Briefly, circulating antibody-secreting cells, Naive, Memory, Non-Switched Memory and Double negative B cells were defined as CD19+/lowCD38hiBlimp1+, IgD+CD27−, IgD-CD27+, IgD+CD27+ and IgD-CD27−, respectively. Manual gating was performed for tumor samples from PANDORE. CD4+ TILs and CD8+ TILs from untreated tumors, were clustered within and across samples using PhenoGraph.

### 3.2. Flow Cytometry

Sample preparation. Cells were stained for viability with Zombie Acqua (BioLegend Cat #423102) for 20 min at +4° C. Cells were washed in staining buffer and incubated during 20 min at room temperature with a panel of antibodies antibodies (table 3). Samples were washed in staining buffer with Brillant Strain Buffer (BD, Cat #563794).

Data Acquisition. Samples were acquired on BD LSRFortessa X-20 Flow Cytometer.

### 4. In Sitro Tumor Assays.

One out of 18 samples was processed after thawing, the others were performed freshly. Dissociated bladder cancers were stained for DO (baseline) with mass cytometry as described in the section.

One hundred thousand of CD45+ cells per well were incubated in complete medium (RPMI 1640 supplemented with 10% human AB serum (Institut Jacques Boy, Cat #201021334), 1% Penicillin/Streptomycin (GIBCO Invitrogen, Cat #15140-122), 1% I-glutamine (GIBCO Life Technologies, Cat #25030-024) and 1% of sodium pyruvate (GIBCO Life Technologies, Cat #11360-039)) in a 96-well U-bottom plate. Isotypes controls (IgG4 and IgG1 (BioLegend, Cat # at 25 μg/mL and 5 μg/mL, respectively), recombinant IL-2 (PeproTech, Cat #200-02-11, at 10 μg/mL), anti-CD3 (Thermo Fisher Scientific, clone OKT3, at 10 μg/mL)+anti-CD28 (Thermo Fisher Scientific, clone CD28.2, at 10 μg/m) were added to control wells. AC anti-PD-1 (pembrolizumab, from Merck, at 25 μg/mL) and AC anti-CD38 (daratumumab, from Janssen-Cilag, at 5 μg/ml) were added to tested wells. Plates were incubated at +37° C. with 5% CO2 for 3 days. After incubation, cells were centrifuged at 1500 rpm for 5 min. The supernatants were collected and stored at −20° C. until measurements. The pellets were resuspended in PBS 1× for flow cytometry staining.

### 5. Immune Response Against Commensals

### 5.1. Bacterial Strains and Culture Conditions.

Cultivable bacteria, relevant in bladder cancer patients, Escherichia coli (Q1696, IHU Marseille, urine from non-cancer patient), Fusobacterium nucleatum (P6429), Bacteroides fragilis (Ileon6), Enterococcus faecalis (GR, urine from cancer patient NR PANDORE), Staphylococcus capitis (feces from kidney cancer patient non-responder to ICBs, EverImmune), Staphylococcus epidermidis (GR, urine from cancer patient), Streptococcus mitis (GR, urine from healthy volunteer), Streptococcus sanguinis (GR, urine from cancer patient) and BCG (Sanofi) were plated onto sheep's blood agar plates (COS, BioMérieux) at 37° C. with 5% CO2 in anaerobic or aerobic conditions for 48 h and identified by Matrix Assisted Laser Desorption Ionization-Time of Flight (MALDI-TOF) mass spectrometry (Andromas; Beckman Coulter, Brea, California USA).

### 5.2. Assessing CD4+ T Cell Memory Responses

The experiments were performed as much as possible in paired setting assessing the reactivity of pre- and post-memory CD4+ T cells during the same experiment.

Generation of monocyte-derived dendritic cells (mo-DC). Frozen PBMC were thawed, washed and resuspended in mo-DC medium (RPMI 1640 supplemented with 10% human AB, 1 mM of glutamine, 1% sodium pyruvate, 1% HEPES, 1% penicillin/streptomycin. Viability and count were evaluated using a Vi-Cell XR Cell Counter. Cells were then cultured at 5M/mL in 24-well flat bottom plates (5M cells/well) for 2 hours at 37° C., 5% CO2 and separated into adherent and non-adherent cell populations. The non-adherent fraction, containing Peripheral Blood Lymphocytes (PBL), was slowly collected, washed and resuspended in PBL media (IMDM medium (Sigma-Aldrich Cat #13390-500 mL, supplemented with 10% human AB, 1 mM Glutamine, 1% sodium pyruvate, 1% HEPES, 1% penicillin/streptomycin and 100 IU/mL rhIL-2 premium grade (Miltenyi, Cat #130-097-745). PBL were seeded in 24-well round bottom plates and cultured for 3 days at 37° C., 5% CO2. The adherent fraction was cultured for 67 hours, at 37° C., 5% CO2 in mo-DC differentiating medium (mo-DC media with GM-CSF (1000 IU/mL, Miltenyi Cat #130-093-867) and IFNα2b (250 IU/mL, Introna®, MSD France). The volume defined to get 5M/mL was used to resuspend PBL and to add on the adherent fraction.

mo-DC-bacteria co-cultures. After incubation, plates containing mo-DC were incubated during 20 minutes on ice. Monocytes-derived DC (adherent and non-adherent fraction) were harvested by flushing with cold mo-DC medium without antibiotics and washed. Cells were then counted using a hemocytometer and the suspension was adjusted to 0.125 M/mL of mo-DC. Five thousand of mo-DC were seeded in 96-well round bottom plates. Bacterial suspensions containing Escherichia coli (IHU Marseille, urine from non-cancer patient), Streptococcus sanguinis (GR, urine from cancer patient) and Staphylococcus capitis (feces from kidney cancer patient non-responder to ICBs, EverImmune) were prepared by adjusting the turbidity at 0.3 MacFarlan in 2 mL of NaCl 0.9% (200 M of bacteria). The suspensions were centrifuged at 4000 rpm for 10 min at +4° C. and resuspended in 4 mL of mo-DC medium without antibiotics to get 50M/mL. A multiplicity of infection (MOI) of 100 was used corresponded to 500,000 bacteria per well. The plates were then centrifuged 2 min at 1000 rpm and incubated at 37° C., 5% CO2 for 2 hours. Wells with mo-DC alone were prepared as negative control. After incubation, mo-DC media with antibiotics was added to each well.

Isolation of memory CD4+ T cells and co-cultures with bacteria. Frozen PBMC obtained before and after pembrolizumab were thawed, washed and resuspended in isolation buffer (PBS 1×+0.5% bovine serum albumin+2 mM EDTA). For some experiments, PBL cultured in IL-2 were pooled with fresh thawed PBMC from the same timepoint. CD4+CD45RO+ T cells were isolated using the memory CD4+ T cells isolating kit (Miltenyi Cat #130-091-893) according to manufacturer's instructions. All the steps were performed on ice. The fraction of memory CD4+ T cell were resuspended at 0.5 M/mL and 100 μL were added to each well containing the mo-DC loaded with bacteria. Memory CD4+ T cells (without mo-DC) alone, or with CD3/CD28 beads (10,000 Dynabeads® per well, Dynabeads T Activator, Thermo Fischer Scientific, Cat #11131D) as negative and positive controls respectively. mo-DC loaded with bacteria (without memory CD4+ T cells) were also cultured.

The co-cultures were incubated for 48 hours at 37° C., 5% CO2. Supernatants were harvested and stored at −20° C. for determination of IFNγ, IL-10 and CXCL13, as measured by commercial ELISA (details in the soluble factor measurement section).

### 5.3. Bacterial Flow Cytometry Assay

Colonies of bacteria were picked, suspended in 1×-PBS-10× glycerol at 109 Colony Forming Unit (CFU)/mL using spectrophotometer (OD(600 nm)=1) and frozen at −80° C. An accurate quantification of CFUs was then performed by counting bacterial events on a flow cytometer (PANDORE study: Cytoflex, Beckman Coulter and validation cohort: Canto, BD).

The IgG and IgA titers were defined by with Optilite® analyser (Binding Site). All buffers were sterilized by filtration with a membrane with 0.22 μm pores. Sera were normalized at 20 μg/mL IgG or IgA in PBS 1×, 2% BSA (SigmaAldrich), 0.02% sodium azide (Sigma-Aldrich). Specific serum antibody levels against purified strains were measured by using a flow cytometry assay, as previously described (Moor et al., 2016). 106 bacterial strains were suspended in PBS 1×, 2% BSA, 0.02% sodium azide in a 96-well V-bottom plate. Twenty-five μL of normalized sera (patients' samples or human normal immunoglobulin (IVIG, Flebogamma®, Instituto Grifols) as a positive control or PBS as a negative control) were added in the 96-well V-bottom plate at a final concentration of 10 μg/mL, and the plates were incubated for 30 min at +4° C. After washing with PBS 1× (10 min, 4,000 g, +4° C.), plates were incubated with secondary conjugated antibodies (1/400e), either a goat anti-human IgA FITC AffiniPureGoat a chain specific or IgG Alexa Fluor®647 (or 488) AffiniPureGoat Fcγ fragment specific or isotype controls as negative controls (all from Jackson ImmunoResearch, Cat #109-095-011, Cat #709-116-073, Cat #009-090-011 and Cat #005-600-003) for 20 min at +4° C. Then, bacteria were washed, fixed in 25 μL paraformaldehyde (4% in PBS 1×) (eBioscience) for 10 min at room temperature and resuspended in sterile PBS. We used 3 different cytometers for 3 independent experiments: samples were run using a Cytoflex (Beckman Coulter for PANDORE study), another Cytoflex (Beckman Coulter for the validation cohort 2) and a Canto (BD for the validation cohort 3), and 30,000 bacterial events were acquired. Analysis was performed with FlowJo software (Tree Star). Medians of fluorescence (MFI) were used to measure mAb-binding levels against the specific strains. For each experiment, we calculated stain index (SI) with sample MFI minus negative control MFI divided by twice standard deviation of batch MFI (SD):

$$SI = \frac{sample MFI patient - negative control MFI}{2 \times S D} .$$

For each experiment, the SD was defined using the isotype control for each plate and each strain of bacteria.

As we performed three independent experiments MFI measurement results in variation due to flow cytometers variabilities. Normalization of the data was performed intra-experiment, but inter-experiment comparisons is therefore impossible.

### 5.4. Whole Blood (WB) Interferon-Gamma, CXCL13, CXCL9, TNFa, IL-10 Release Assay to Monitor Cell-Mediated Immunity to Bacteria

This assay is illustrated in FIG. 18 and FIG. 24.

Pasteurized bacteria were prepared as follows: suspension of bacteria at concentration of 10⁹ bacteria/ml were heat-inactivated 30 min at 70° C. then stored at −80° C. Frozen heat-inactivated bacteria were thawed at 4° C. then diluted on ice in NaCl 0.9% (Versylene Fresenius) for final concentrations 1.5×10⁴ bacteria/ml and 0.5×10⁴ bacteria/ml.

Blood samples from donors were collected in heparinized tubes then diluted 1:2 with a solution of heparin (PanPharma) at [17 UI/ml] and 180 μl of this solution were seeded in replicate wells plated in 96-wells flat bottom plates.

1:2 diluted heparinized whole blood was then stimulated under different conditions: (i) [25 ng/ml] PMA (CalbioChem) [100 ng/ml] inomycine (Sigma); +/−bacteria (ii) bacteria alone (iii) unstimulated (iv) [100 ng/ml] LPS and [10 ng/ml] LPS (Invivogen); (iii) and (iv) were used as negative and positive controls, respectively.

Specificity of cell-mediated immunity was assessed by adding for each condition an anti-class-I or anti-class II MHC inhibitor (anti-HLA-ABC or DP-DQ-DR respectively) [10 μg/ml](Biolegend).

For each condition, the volume was completed to 210 μl/well with NaCl 0.9% then the plates were incubated at 37° C., 5% CO₂. After 22-24 Hrs of incubation, the plates were centrifuged 5 min at 450 g, and supernatants collected then frozen at −20° C.

5.5. Whole Blood Reactivity Against Peptides Derived from E. coli Peptide

Same procedure as the one described in 5.4., but incubated 10 ug/ml of each peptide pool. Myelin basic protein (MOG) was used as a negative.

### 6. Soluble Factors Measurements

### 6.1. Multiplex Immunoassays.

Plasma from PANDORE were thawed at +4° C. overnight then centrifuged at for 15 min at 1000×g. Plasma were monitored using Bio-Plex Pro™ Human Cytokine 40-plex Assay (Bio-Rad, Cat #71AK99MR2) according to the manufacturer's instructions. Supernatants from in sitro assays were monitored using Bio-Plex Pro™ Human Cytokine 27-plex Assay (Bio-Rad, Cat #M500KCAF0Y) according to the manufacturer's instructions. Acquisitions and analyses were performed on a Bio-Plex 200 system (Bio-Rad) and a Bio-Plex Manager 6.1 Software (Bio-Rad), respectively.

### 6.2. ELISA.

CXCL13 in the in sitro supernatants was detected using the Human CXCL13/BLC/BCA-1 Quantikine ELISA Kit (R&D system, Cat #DCX130) according to the manufacturers' instructions. Supernatants from in vitro stimulations with bacteria were monitored using the Human CXCL13/BLC/BCA-1 Quantikine ELISA Kit (R&D system, Cat #DCX130), the ELISA MAX™ Deluxe Set Human IFNγ (BioLegend, Cat #430116) and ELISA MAX™ Deluxe Set Human IL-10 (BioLegend, Cat #430604) according to manufacturer's instructions.

For the whole blood reactivities: IFNg was quantified from supernatants according the manufacturer recommendations, using Biolegend ELISA MAX deluxe set. The release of CXCL13 was assessed using Bio-plex (Biorad) Luminex technology.

### 7. Tissue Analyses

### 7.1. Immunohistochemistry, Multiplex Immunofluorescence, and PD-L1 Assessment

Formalin-fixed paraffin-embedded (FFPE) samples corresponding to representative slides of TURBT and cystectomies with invasive urothelial carcinoma were sequentially cut at 3 μm of thickness. Hematoxyline-Eosine-Safran (HES) staining was performed in the first cut, so immunohistochemistry for CD20 and multiplex immunofluorescences could be performed in the subsequent ones. CD20 immunochemistry was performed by an anti-CD20 (DAKO, Cat #M0755, 1/100 dilution, incubation at 36° C. for 20 min) on a Benchmark Ultra (Roche) autostainer system. Briefly, FFPE sections were deparaffinized and subsequently antigen-retrieval was conducted with CC1 Buffer (pH=8) for 36 min. CD20 was revealed with ultraView Universal DAB kit. Lipopolysaccharide (LPS) immunochemistry was performed by an anti-LPS (HycultBiotech, Cat #HM6011, 1/6000 dilution, incubation at 37° C. for 30 min) on a BOND-RX (Leica) autostainer system. Briefly, FFPE sections were deparaffinized and subsequently antigen-retrieval was conducted with ER1 Buffer (pH=6) for 30 min. The primary antibody was revealed with Refine DAB kit. Triplex immunofluorescences were performed on a BOND-RX (Leica) autostainer system and a OPAL (Akoya) system. In short, FFPE sections were deparaffinized and subsequently antigen-retrieval was conducted with ER2 Buffer (pH=9) for 20 min. First, antibodies were added starting with PD-1 (R&D System, Cat #AF1086, 1/50 dilution, incubation at room temperature for 30 min), CD38 (Cell Signaling Technology, Cat #51000, 1/6000, incubation at 37° C. for 30 min) and then CD4 (Spring, Cat #M3364, 1/400, incubation at room temperature for 30 min). Staining was performed using the OPAL polymer (except for PD-1 which was revealed with anti-goat polymer Polink-1, GBI Labs) with OPAL520 (1/400), OPAL570 (1/1200) and OPAL690 (1/50), respectively. Second, antibodies were added starting with CD38 (Cell Signaling Technology, Cat #51000, 1/6000, incubation at 37° C. for 30 min), IgG4 (Epitomics, Cat #AC-0148, 1/3000, incubation at 37° C. for 30 min), CD4 (Spring, Cat #M3364, 1/400, incubation at room temperature for 30 min). Staining was performed using the OPAL polymer with OPAL570 (1/500), OPAL520 (1/100) and OPAL690 (1/100), respectively. Baseline PD-L1 immunohistochemistry was performed by an anti-PD-L1 IHC 22C3 (DAKO, Cat #M3653, 1/25 dilution, incubation at room temperature for 30 min) on a BOND-RX (Leica) Autostainer system. In short, FFPE sections were deparaffinized and subsequently antigen-retrieval was conducted with ER2 Buffer (pH=9) for 20 min. The PD-L1 antibody was revealed with Refine DAB kit. An experienced pathologist (J-Y.S.) determined the CPS, and PD-L1 positivity was qualified as CPS 10.

### 7.2. Evaluation of Tertiary Lymphoid Structures (TLS)

Microscopic examination of HES stained TURBT and cystectomies from patients with invasive urothelial carcinoma (IUC) was performed by a pathologist (L.L.) in order to morphologically identify TLS (lymphoid aggregates, primary follicle-like, and secondary follicle-like). Lymphoid aggregates were determined as vaguely nodular aggregates of small non-cleaved lymphoid cells, usually with more than 250 and less than 500 cells, without two or more of the early germinal center elements (follicular dendritic cells, centroblasts, mantle zone or high endothelial veins); primary follicle-like tertiary lymphoid structures were determined as nodular aggregates of predominantly small lymphoid cells, usually with more than 500 cells, with three or more of the follicular elements, but without late germinal center elements (tinged body macrophages or dark/light areas of a clear germinal center, marginal zone); secondary follicle-like tertiary lymphoid structures were determined as lymphoid follicles showing late germinal center structures.

### 7.3. Evaluation of CD20

Evaluation of CD20 was available in order to determine presence of B cells in more than 50% in lymphoid aggregates. Whole slide image (WSI) at magnification of 20× and 40× were obtained with Olympus scanner VS120, respectively, for HES and CD20 stainings for TURBT and cystectomies. The images were analyzed by QuPath software, version 0.2.3. TLS structures were annotated in WSI in order to determine the best fit for CD20 positive dense areas. Using simple tissue detection tool (threshold 165, requested pixel size 15, minimum area 104 μm2, max fill area 106 μm2, smooth image, cleanup with median filter, expand boundaries, and smooth coordinates), TLS areas were automatically annotated, while artifact areas were manually excluded by a pathologist (L.L.). The percentage of TLS area was calculated based on whole tissue area, which had been also annotated in the WSI. Also, tumor areas were manually annotated on CD20 slides, based on HES, by a pathologist (L.L.).

### 7.4. Evaluation of CD4+CD38+PD-1+ and CD4+CD38+IgG4+ Populations in FFPE Samples

Immunofluorescence was performed for identification of triple staining corresponding to CD4+CD38+PD-1+ and CD4+CD38+IgG4+ cells in TURBT and cystectomies. WSI were obtained with Olympus scanner VS120 for immunofluorescence triple staining (exposure time: 5 ms for DAPI, 30 ms FITC, 200 ms CY3 and CY5).

### 7.5. Geodistribution of CD4+CD38+PD-1+ and CD4+CD38+IgG4+ in FFPE Samples

Tumor and TLS annotated areas on CD20 slides were matched to density maps generated on WSI from triplex stainings on QuPath in order to define where CD4+CD38+PD-1+ and CD4+CD38+IgG4+ populations were located. Populations identified with the IgG4-triplex were counted within 3 TLS areas. L.L. delineated the TLS areas.

### 7.6. Quantification and Phenotyping of Cells

The images were analyzed by QuPath software, version 0.2.3 (Bankhead et al, 2017). For each panel (CD38/CD4/PD-1 and CD38/CD4/IgG4), 3 different classifiers, based on fluorescence intensities, were then combined to phenotype the cells. In TLS areas, cells were detected on DAPI channel with the Cell Detection function. The resulting densities are expressed as a number of cells of each phenotype per square millimeter of tissue.

### 7.7. Determination of Cell Distance to its Closest TLS

Quantifications were performed using R, by means of the EBImage, RBioFormats & RniftyReg packages. TLS masks were created by applying an affine transformation to the manually annotated mask created from CD20 staining. Of note, the affine parameters were determined using image registration on automatically-generated tissue masks (thresholding the DAPI image) from both CD20 and triplex stainings.

Distance maps were then computed from the TLS masks, indicating for each pixel the distance to the closest object pixel. Finally, cell shortest distances to TLS were extracted by selecting the map pixel value at their centroid coordinates.

### 7.8. Immunoscore IC™ and Assessment of CD3+ and CD8+ Cells

Immunohistochemistry. Immunoscore IC™ (Veracyte, Marseilles, France) is an assay designed to measure the densities of PD-L1+ and CD8+ cells as well as the proximity between these cells on a single tissue section with image analysis tools. Immunohistochemistry-based staining was performed on Benchmark XT instrument (Roche-Ventana) as follows: standard deparaffinization, Cell Conditioning 1 for 54 min, anti-PD-L1 (clone HDX3) incubation at 37° C. for 60 min, anti-CD8 (clone HDX1) incubation at 37° C. for 60 min, and Hematoxilyn II counterstaining for 8 min. Anti-PD-L1 and anti-CD8 antibodies were revealed with OptiView DAB IHC Detection Kit and ultraView Universal Alkaline Phosphatase Red Detection Kit respectively. Immune infiltration of tumors by CD8+ and CD3+ cells was assessed on two adjacent tissue sections followed by Digital Pathology analysis with a dedicated software. Staining was adapted from http://dx.doi.org/10.1136/jitc-2019-000272.

Digitization. Every stained slide was scanned with a high-resolution scanner (NanoZoomer XR, Hamamatsu) to obtain 20× digital images for subsequent analysis by digital Pathology.

Digital Pathology. Whole slide images were analyzed on HALO platform (Indica labs, Corrales, NM, USA) and consisted in, 1) detection of the tissue section and definition of tumor core, 2) detection and quantification of stained cells. Subsequently, cells coordinates and phenotypes were exported to analyze their spatial distribution.

Main computed variables were, CD3+ cell density (cells/mm²), CD8+ cell density, PD-L1+ cell density, proximity between CD8+ and PD-L1+ cells, clustering of CD8+ or PD-L1+ cells. Arbitrarily, cut off distances used to compute proximity and cluster indexes was set to 20 μm.

### 7.9. Evaluation of Bacteria in FFPE Samples

Microscopic evaluation of morphological structures that could indicate the presence of bacteria in TURBT samples was performed by a pathologist (L.L.). Cystic-like structures, sometimes present in cytoplasmic vacuoles filled with round or ovoid basophilic elements were identified in non-tumoral urothelial epithelium, but also in tumor cells corresponding to IUC. Immunohistochemistry for LPS was performed in order to identify occasional presence of bacteria in the samples. A group of at least 5 positive cells showing granular cytoplasmic staining in tumor cells corresponding to IUC was expected to the case to be considered positive. Also, normal urothelial epithelium, immune cells, and cystic-like structures were evaluated for LPS staining, when present.

8. Fluorescence In Situ Hybridization for Uropathogenic Escherichia coli

De-paraffining. FFPE samples corresponding to representative slides of TURBT with invasive urothelial carcinoma were sequentially cut at 3 μm of thickness. Slides were deparaffinized in the hybridizer for 90 minutes at 60° C., then incubated for 10 minutes in successive solutions of Clearify 100%, ethanol 100%, 70%, 50%, 25%, and finally distilled sterile water.

FISH. It was carried out as previously described, with the following modifications (Grine et al, 2018). For permeabilization of the tissues, slides were incubated for 5 min in a proteinase K solution (10 μg/ml) at 56° C., followed by a distilled water rinsing step. Hybridization was performed at 60° C. for three hours using 5′-3′ probes for chuA (GCTACCGCGATAACTGTCAT and TGGAGAACCGTTCCACTCTA), c3686 (TTGCACCAACAACGTCTACC and TCTGCGTCTTCTACCATCAC) and c3509 (ACAATCCGCCACCATCCAG and CTCTCCACCGGAGAGTGTT) specifically targeting UPEC (Brons et al, 2020), coupled to Alexa-488 fluorochrome (Eurogentec, France). Excess probes were removed by rinsing for 5 minutes in degrading series dilutions of saline-sodium citrate solution (4×, 2×, 1×, 0.5×, water). Slides were then air-dried at room temperature in the dark. DAPI has been used for nuclear staining. the slides were observed using the confocal microscope LSM800 with two excitation wavelengths: 493 nm for the UPEC-specific signal, and 353 nm for the DAPI stain.

### 9. Transmission Electron Microscopy

Biopsies were performed in area of interest from FFPE tissues and processed as described (Graham et al, 2007). After rehydration, samples were kept for 4 days in glutaraldehyde 2.5%, HEPES 0.1M at 4° C. Then, samples were washed 3 times with PHEM 1×, for 10 min. A second fixation were performed with OsO4 2% and potassium ferricyanide 1.5% in water for 2 h. Then, samples were washed 3 times with water. Dehydration was performed with continuous agitation using Ethanol 25, 50, 75 and 95%, 15 min, Ethanol 100% for 20 min, 3 times followed by propylene oxide, 15 min. Then, incubation with propylene oxide/Epon A+B+DMP30 using continuous agitation overnight, then biopsies were washed 3 times with Epon A+B+DMP30 for 2 h of continuous agitation. Samples were put at 60° C. for 48 h. Sections with a thickness of 70 nm were cut with a Leica UCT microtome and collected on carbon, formvar coated copper grids. Sections were contrasted with 4% aqueous uranylacetate and Reynold's lead citrate. Stained sections were observed with a Tecnai spirit FEI operated at 120 kV. Images were acquired with FEI Eagle digital camera.

10. Bacteria Identification by qPCR Detection

Total genomic DNA from bladder tissues was extracted with DNeasy Blood & Tissue Kit (Qiagen, Cat #69506) following manufacturer's recommendation using spin-column purification. Measurement of extracted DNA was performed using Nanodrop and normalized to 10 ng/μL. Genomic DNA was analyzed by PowerUp SYBR Green Master Mix (Invitrogen) according to the manufacturer's instructions (2′ 50° C., 10′ 95° C., 45 cycles with 15″ 95° C. and 1′ 60° C. followed by 15″ 95° C., 1′ 60° C. and 15″ 95° C.) using Quant Studio 3 (Applied Biosystems). Expression was normalized to the expression of the total bacterial load determined by Universal 16S RNA gene by means of the 2-ΔCt method. All primers were from Thermo Fisher Scientific. We used: ACT-CCT-ACG-GGA-GGC-AGC-AGT (all bacteria-F), ATT-ACC-GCG-GCT-GCT-GGC (all bacteria-R), CATGCCGCGTGTATGAAGAA (E. coli-F) and CGGGTAACGTCAATGAGCAAA (E. coli-R).

### 11. Culturomics of MIBC

A trained pathologist (C.R.) processed the tissues in sterile conditions.

The following operations were carried out in a laboratory with Biological Safety Level 2 (NSB2) and under a class II Microbiological Safety Station (MSP), in order to avoid and limit any contamination. All materials were disinfected with 70° C. alcohol.

### 11.1 Sample Preparation

Partitioning. When identifiable, we separated the fresh tissue into layers (muscle, fat, mucosa) from the tumour and the non-tumour tissues. RPMI used for the transport of the tissues was also analysed separately.

Processing. Pestle Motor Mixer (Argos Technologies) and Homogen Sys 1.5 mL Pestle (SP Scienceware, Wayne, USA) was used to mix tissue samples.

We splitted each sample in 1 g of tissue per 5 ml of NaCl 0.9%.

### 11.2. Culture Conditions

Solid culture using plates. 100 μl of each sample was diluted with 900 μl of NaCl. 1/10th and 1/100th dilutions were performed. A total volume of 200 μl of each dilution was used for culturing on directly on 5% sheep blood-enriched columbia agar (COS) (BioMérieux, Marcy l'Etoile, France). Plates were incubated in aerobic and anaerobic atmospheres at 37° C.

Liquid culture. Aerobic and anaerobic blood culture bottle (BD BACTEC Plus Aerobic medium, Dun Laoghaire, Ireland and BD BACTEC Lytic Anaerobic medium, Dun Laoghaire, Ireland) supplemented with sterile a 0.2 μm-filtered rumen (3 mL) and sheep blood defibrinated (Oxoid Limited, Hampshire, England) (3 mL) were used. Bottles were incubated at 28° C. and 37° C. For each sample, a total volume of 1 mL was inoculated into each of the two bottles using a syringe needle. As controls, supplemented bottles (without samples) were used as negative controls. These vials were then processed according to the specimen to check for any contamination.

The anaerobic atmosphere was generated using an anaerobic jar (W-Zip PlasticPouches, Oxoid Limited, Hampshire, England) and an atmosphere generator (GENbox aner, bioMérieux, Marcy l'Etoile, France).

### 11.3. Isolation and Identification

Isolation. COS plates were routinely checked and CFU were identified as below. For liquid cultures, on day 1, 3, 7, 10, 15, 20, a total volume of 100 μl of each sample from BD BACTEC vials was diluted with 900 μl of NaCL 0.9%. A dilution series ranging from 1/10 to 1/100 were performed. A total volume of 200 μl of each dilution was culture COS plates and incubated under the same conditions as the initial bottle. Anaerobic conditions were cultivated for 5 days whereas aerobic conditions were cultivated for 3 days. The colonies were manually sub-cultured onto COS plates for MALDI-TOF MS identification.

Strain identification by MALDI-TOF MS. Bacterial colonies were identified using Matrix Assisted Laser Desorption/ionization Time-Of-Flight (MALDI-TOF) mass spectrometry (Bruker France Daltonics, France). Each deposit was manually performed and then covered with 2 μL of a matrix solution (HCCA-portioned-Matrix for Maldi-Tof-MS measurements in microorganism identification—(Bremen, Germany) with 500 μL of Solution OS (Acetonitrile 50%/Water 47.5%/Trifluoroacetic acid 2.5%) (LCH CHIMIE, Les Aires, France)). This analysis was performed using MicroFlex mass spectrometer (Bruker France Daltonics, France) according to the manufacturer's recommendations. The acquired spectrum was then loaded into the MALDI Biotyper Software (Bruker France Daltonics) and analyzed, by using the standard pattern-matching algorithm, which compared the spectrum acquired with that present in the library (Bruker database, constantly updated). Score values of ≥1.7 but <2 indicated identification beyond the genus level, and score values of ≥2.0 indicated identification at the species level. Scores of <1.7 were interpreted as not relevant. An isolate was labelled as correctly identified at the species level when at least one of the colonies spectra had a score ≥1.9 and one other of the colonies spectra had a score ≥1.7 (Seng P et al., 2009, Clin Infect Dis Off Publ Infect Dis Soc Am).

12. DNA Extraction, Libraries and Analyses from TURBT Samples

Six slides of FFPE tissues (TURBT samples) of 10 μm were used per patient.

### 12.1. DNA Extraction.

The FFPE samples were extracted with the Maxwell Promega for the extraction of genomic DNA with the Maxwell® RSC DNA FFPE Kit (Promega, Cat #AS1450) according to the manufacturer's instructions. The proteinase K treatment was done overnight at 56° C. and finalizing at 80° C. for 4 hours. Samples were then treated with RNAse before the extraction process.

### 12.2. Libraries.

The panel targets all tumor suppressors or oncogenes covering the complete sequencing of the codifying region of a total of 411 genes identifies point mutations, including single-nucleotide variants and small indels. The detection of copy number variations (gains and losses of chromosomes or complete chromosomal arms) throughout the genome to specific genes and even exons was based on the coverage ratio. The total size of this panel is 1,739,310-pb. The custom panel uses the SureSelect XT HS kit (Agilent) designed for small amounts of FFPE DNA as input and detect low allelic frequencies. After the enzymatic fragmentation of 50-200 ng of tumoral gDNA from each sample to an average of 200-400 bases The full preparation was done on a Bravo equipment option B (Agilent). Library quality control was performed on Tapestation 2000 and the commercial kits D1000 reagents and D1000 ScreenTape (Agilent). DNA samples were end-repaired, dA-tailed and ligated to the molecular-barcoded adaptor system and purified using AMPure XP beads. Pooled libraries containing captured DNA fragments were subsequently sequenced on an NovaSeq 6000 platform (Illumina) as 2×150-bp paired-end reads. Sequences were demultiplexed using an in-house tool.

### 12.3. Analysis.

The data analysis pipeline included the following algorithms developed internally: BWA-MEM v-0.7.12 for read alignment to the hg19 human reference genome and Samtools v-1.2 and Picard-tools v-1.139 for PCR duplicate quantification and removal. GATK Haplotype v-3.4-46, snpEff v-4.0 and MutaCaller-1.7 (home pileup internally developed) were used for variant calling and classification. Variants were called with a minimum allelic frequency threshold of 1% for already classified variants (those known in the internal database) and 5% for non-classified variants, and a read depth threshold of 30× for the total reads at the variant location and at least 10× for the variant.

Several filters were applied to further select for potentially relevant variants among the called variants. The population databases Exac and gnomAd were used to automatically filter out polymorphism as soon as the population frequency was higher than 0.5%. Non-classified variants (not known in the internal database) were excluded if the intra-run recurrency was superior to 4.

The tumor mutational load was assessed with the Mercury solution (Integragen, France) calculated by dividing the number of somatic mutations by the number of bases having a depth greater than 10. We used the data published by Lawrence et al., 2013. The somatic mutations used for the mutational load are filtered as follows: Somatic score >3, Mutated Allele Frequency in Tumor tissue ≥5%, Mutated Allele Count in Tumor tissue ≥3, population heterozygous internal database frequency ≤1%, population homozygous internal database frequency ≤1% and EVS & 100 G & Exac variant frequency ≤0.5% and consequences on protein: Stop, Start, Missense, Splice for the SNPs and Inframe, Frameshift for the indels.

### 13. Single-Cell RNA Sequencing

Frozen samples were thawed in complete RPMI, counted and washed in PBS 1×+0.5% bovine serum albumin+2 mM EDTA. The isolation and enrichment of CD45+ were performed using the REAlease® CD45 (TIL) MicroBead Kit, human (Miltenyi, Cat #130-121-563) according to manufacturer's instructions.

For the Rhapsody experiment, all the process was done by following manufacturer's (BD Biosciences) protocol. 2,432 cells were captured in a single run with 7 barcoded samples pooled together. The sample was processed according to BD mRNA targeted and sample tag library preparation with the BD Rhapsody™ targeted mRNA and Abseq amplification kit (Doc ID: 210969 Rev 3.0). The BD Rhapsody™ Immune Response Targeted Panel (Human) (BD, Cat #633750) was used. Samples were then subjected to an indexed paired-end sequencing run of 2×151 cycles on an Illumina HiSeq 4000 system (Illumina, San Diego, CA, USA) with 20% PhiX spike in.

Targeted transcriptomics Fastq files were processed via the standard Rhapsody analysis pipeline (BD Biosciences)) per the manufacturer's recommendations. First, R1 and R2 reads are filtered for high-quality reads, dropping reads too short (less than 66 bases for R1 and 64 bases for R2) or have a base quality score of less than 20. R1 reads are annotated to identify cell label sequences and unique molecular identifiers (UMIs), and R2 reads are mapped to the respective reference sequences using Bowtie2. Finally, all passing R1 and R2 reads are combined and annotated to the respective molecules. For quality control of the reads, recursive substation error correction (RSEC) and distribution-based error correction (DBEC) were applied, which are manufacturer-developed algorithms correcting for PCR and sequencing errors. For determining putative cells (which will contain many more reads than noise cell labels), a filtering algorithm takes the number of DBEC-corrected reads into account, calculating the minimum second derivative along with the cumulative reads as the cut-off point. Finally, the expression matrix was obtained from the DBEC-adjusted molecule counts in a CSV format.

A cell was determined as a singlet if the minimum read count of a single sample tag is above the threshold of 75%. A cell was classified as a multiplet if the cell exceeds the threshold for more than one sample tag. A cell that does not meet the threshold was labelled as undetermined. Both multiplets and undetermined cells were excluded from the analysis as described below.

For downstream analysis in Seurat V4 (Hao, Satija Cell 2020). Counts were normalized and PCA analysis was performed. To incorporate protein information into the analysis, the Abseq signals were added to the variable genes found by the “FindVariableFeatures” function. Using these variable features and proteins, a UMAP was generated. Clustering was performed using SNN nearest-neighbour analysis. Differentially expressed proteins and genes were identified as FDR <0.05 and log FC below −0.25 or above 0.25.

### 14. Analyses and General Statistical Analysis

Exploratory analyses were performed comparing PFS according to biomarkers status by using the log-rank tests and p-values were not corrected for multiple testing.

Flow cytometry and mass cytometry analyses were performed with FlowJo software (Tree Star). Data representations and analyses were performed either with Prism 7 (GraphPad San Diego, CA, USA) or R v3.6 using readxl, tidyverse, dplyr, ggplot2, ggpubr, ggsignif, pheatmap, corrplot, ggdendro, Hmisc, heatmapply and survminer packages. Heatmaps of normalized marker expression (CyTOF) were generated using heatmapply R package. For in sitro assay, soluble factors fold ratios were calculated as log 2 transformation of median values of stimulated versus unstimulated wells and were converted to z scores. Heatmap was generated with the R package Pheatmap. Hierarchical clustering based on the z score was performed using Euclidean distance and ward D2 clustering.

### Example 1: Clinical Efficacy of Pembrolizumab in the PANDORE Study

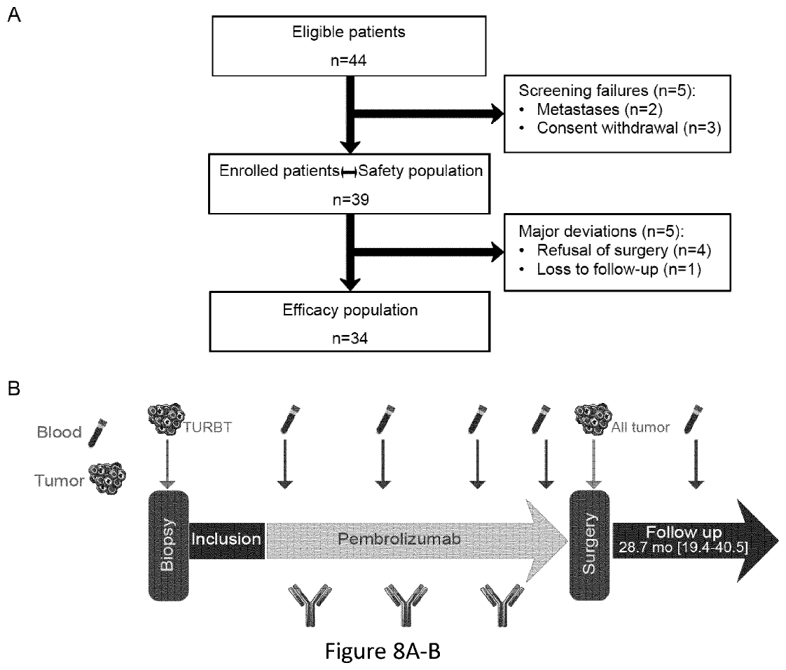

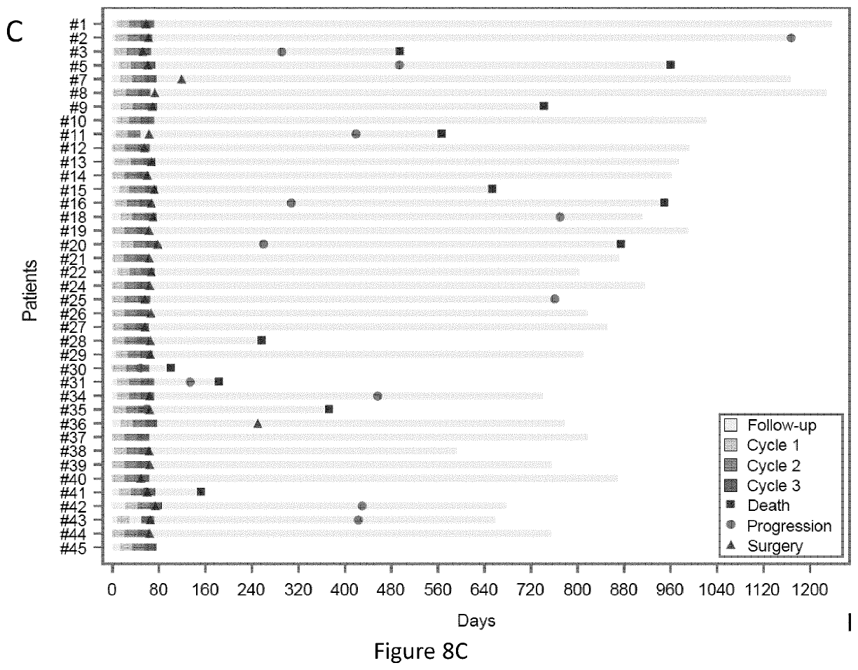

Between October 2017 and May 2019, we enrolled 44 cisplatin-ineligible patients with localized MIBC in the PANDORE trial (NCT03212651) but only 39 patients were included in the final analysis (FIG. 8A-B). Among these patients, 25 patients (64%) met cisplatin-ineligibility and 14 (36%) declined chemotherapy. Following baseline transurethral resection of bladder tumor (TURBT), 39 patients were then treated with pembrolizumab (200 mg) every three weeks for a total of three cycles before radical cystectomy (FIG. 8B). Clinical and tumor characteristics are summarized in Table 1 and FIG. 1. All tumors were urothelial carcinoma. Briefly, most patients were men (74%), with a median age of 72 years (interquartile rage (IQR): 67-78) and presented a cT2 (82%) and a cN0 stages (89.7%) on TURBT samples. Nearly all patients (n=37/39) received the planned three cycles of pembrolizumab. Patient #011 did not receive the last cycle due to grade 2 keratitis and patient #043 did not receive cycle 2 due to worsening adrenal insufficiency and fatigue. Sixty two percent of individuals experienced a pembrolizumab-related adverse event, mostly grade 1 (Table 2). No deaths related to therapy occurred. Median interval between the first infusion and radical cystectomy was 60 days (IQR: 55-63). Out of 39 patients, 34 patients proceeded with radical cystectomy (efficacy population, FIG. 8C). Median time between the last cycle and radical cystectomy was 16 days (IQR: 13-21).

Radical cystectomy by open surgery was performed in 12 patients (35%). Post-surgical complications are detailed in Material & Methods and were similar to those reported in other published studies (Briganti et al., 2020; Szabados et al., 2021). The primary endpoint was the pCR defined as the absence of cancer cells in the bladder and the absence of microscopic lymph node metastases in the cystectomy specimen (ypT0N0). The overall pCR rate was 29.4% (95% confidence interval (CI):15.1-47.5) and the overall downstaging [i.e, ypT0N0, ypTaN0, ypT1N0 and ypTisN0 referred to as major pathological response (MPR)] was 41.2% (95% CI: 24.6-59.3) (not shown).

TABLE 1
Patient baseline characteristics

| | | | | | | | Major | |
|---|---|---|---|---|---|---|---|---|
| | Safety | | Efficacy | | Non- | | pathological | |
| | population | | population | | responders | | response | |
| | (n = 39) | | (n = 34) | | (n = 20) | | (n = 14) | |
| Age (years), median (IQR) | 72 | (67-78) | 72 | (69-78) | 72.5 | (68-79) | 72 | (69-78) |
| Male sex, n (%) | 29 | (74.4) | 24 | (70.6) | 12 | (60) | 12 | (85.7) |

TNM stage, n (%)

| | | | | | | | | |
|---|---|---|---|---|---|---|---|---|
| T2 | 32 | (82.1) | 29 | (85.3) | 16 | (80) | 13 | (92.9) |
| T3 | 1 | (2.6) | 0 | (0) | 0 | (0) | 0 | (0) |
| T4 | 4 | (10.2) | 3 | (8.8) | 3 | (15) | 0 | (0) |
| Not defined | 2 | (5.1) | 2 | (5.9) | 1 | (5) | 1 | (7.1) |
| Previous BCG, n (%) | 7 | (17.9) | 7 | (20.6) | 16 | (30) | 1 | (7.1) |

PD-L1 status

| | | | | | | | | |
|---|---|---|---|---|---|---|---|---|
| PD-L1 CPS, n (%) | 35 | (89.7) | 31 | (91.2) | 20 | (100) | 11 | (78.6) |
| Mean CPS (min-max) | 11.6 | (0-100) | 13 | (0-100) | 14.5 | (0-100) | 10.3 | (0-57.5) |
| CPS ≥ 10 | 5 | (14.3) | 5 | (16.1) | 3 | (15) | 2 | (18.2) |
| CPS < 10 | 30 | (85.7) | 26 | (83.9) | 17 | (85) | 9 | (81.8) |

Intratumoral CD8⁺

| | | | | | | | | |
|---|---|---|---|---|---|---|---|---|
| Intratumoral CD8⁺ | 34 | (87.2) | 30 | (88.2) | 20 | (100) | 10 | (71.4) |
| cell count, n (%) | | | | | | | | |
| Median (cells/mm²) | 303.8 | (103.8-646.7) | 282.3 | (94-714.9) | 179 | (87-578.5) | 398.9 | (113-974.5) |
| (IQR) | | | | | | | | |

ECOG performance status, n (%)

| | | | | | | | | |
|---|---|---|---|---|---|---|---|---|
| 0 | 23 | (60.5) | 20 | (58.8) | 12 | (60) | 8 | (57.1) |
| 1 | 15 | (39.5) | 14 | (41.2) | 8 | (40) | 6 | (42.9) |
| Unknown | 1 | | 0 | (0) | 0 | (0) | 0 | (0) |

Progression-free survival (PFS)

| | | | | | | | |
|---|---|---|---|---|---|---|---|
| Death or relapse, n (%) | n/a | 16 | (47.1) | 14 | (70) | 2 | (14.3) |
| Median (IQR) | n/a | 24.9 | (13.6-30.5) | 18.5 | (10.8-28) | 27.5 | (25-34) |

Overall survival

| | | | | | | | |
|---|---|---|---|---|---|---|---|
| Death, n (%) | n/a | 10 | (29.4) | 9 | (45) | 1 | (7.1) |
| Median (IQR) | n/a | 26.7 | (21.5-31.3) | 25.5 | (21.2-30.7) | 27.5 | (25-34) |

Translational research, baseline

| Peripheral Tfh, n (%) | n/a | 31 | | 18 | (90) | 13 | (93) |
|---|---|---|---|---|---|---|---|
| Peripheral Tfh | n/a | 3.1 | (2-4.7) | 2.8 | (1.9-4.5) | 4.0 | (1.9-6.4) |
| median (% in | | | | | | | |
| CD4⁺) (IQR) | | | | | | | |
| IgG anti-Esherichia | n/a | 33 | | 19 | (95) | 14 | (100) |
| coli, n (%) | | | | | | | |
| IgG anti-Esherichia | n/a | 0.227 | (0.185-0.256) | 0.202 | (0.185-0.252) | 0.246 | (0.185-0.2692) |
| coli, median (SI) | | | | | | | |
| (IQR) | | | | | | | |
| IgG anti- | n/a | 33 | | 19 | (95) | 14 | (100) |
| Staphylococcus | | | | | | | |
| capitis, n (%) | | | | | | | |
| IgG anti- | n/a | 0.0024 | (0.0003-0.004) | 0.003 | (0.002-0.007) | 0.001 | (0-0.003) |
| Staphylococcus | | | | | | | |
| capitis, median | | | | | | | |
| (SI) (IQR) | | | | | | | |

Translational research, dynamic between pre-treatment and cycle 1

| Circulating CXCL13, n (%) | n/a | 23 | | 13 | (65) | 10 | (71) |
|---|---|---|---|---|---|---|---|
| Circulating CXCL13, | n/a | 1.4 | (1.1-1.8) | 1.4 | (0.9-2) | 1.3 | (1.2-1.6) |
| ratio (pre-/C1) (IQR) | | | | | | | |

Translational research, post-treatment

| Intra-tumoral TLS, n (%) | n/a | 33 | | 20 | (100) | 13 | (93) |
|---|---|---|---|---|---|---|---|
| Intra-tumoral TLS, | n/a | 32.6 | (7.4-66.2) | 36.5 | (7.1-68.3) | 32.6 | (5.7-53.1) |
| median (/mm²) (IQR) | | | | | | | |

| | | PFS < 24 | | PFS ≥ 24 | | |
|---|---|---|---|---|---|---|
| | | months | | months | | |
| | P-value | (n = 13) | | (n = 20) | | P-value |
| Age (years), median (IQR) | 0.66⁺ | 72 | (66-78) | 72 | (70-79) | 0.75⁺ |
| Male sex, n (%) | 0.14 | 7 | (53.8) | 16 | (80) | 0.11 |
| TNM stage, n (%) | | | | | | |
| T2 | 0.56 | 13 | (100) | 15 | (75) | 0.15 |
| T3 | | 0 | (0) | 0 | (0) | |
| T4 | | 0 | (0) | 3 | (15) | |
| Not defined | | 0 | (0) | 2 | (10) | |
| Previous BCG, n (%) | 0.10 | 4 | (30.8) | 3 | (15) | 0.28 |
| PD-L1 status | | | | | | |
| PD-L1 CPS, n (%) | | 13 | (100) | 18 | (90) | |
| Mean CPS (min-max) | 0.72⁺ | 14.2 | (0-100) | 12.1 | (0-100) | 1⁺ |
| CPS ≥ 10 | 0.82 | 2 | (15.4) | 3 | (17) | 0.92 |
| CPS < 10 | | 11 | (84.6) | 15 | (83) | |
| Intratumoral CD8⁺ | | | | | | |
| Intratumoral CD8⁺ | | 13 | (100) | 17 | (85) | |
| cell count, n (%) | | | | | | |
| Median (cells/mm²) | 0.25⁺ | 117.1 | (54.2-351.9) | 373.8 | (123.4-796) | 0.07⁺ |
| (IQR) | | | | | | |
| ECOG performance status, n (%) | | | | | | |
| 0 | 0.87 | 6 | (46.2) | 13 | (65) | 0.28 |
| 1 | | 7 | (53.8) | 7 | (35) | |
| Unknown | | 0 | (0) | 0 | (0) | |
| Progression-free survival (PFS) | | | | | | |
| Death or relapse, n (%) | | 13 | (100) | 3 | (14.3) | |
| Median (IQR) | 0.002^{#} | 13.4 | (8.2-15.3) | 28.6 | (25.1-32.7) | <0.0001^{#} |
| Overall survival | | | | | | |
| Death, n (%) | | 10 | | 0 | | |
| Median (IQR) | 0.02^{#} | 21.4 | (14.2-26.2) | 28.7 | (25.7-32.7) | <0.0001^{#} |
| Translational research, baseline | | | | | | |
| Peripheral Tfh, n (%) | 0.22⁺ | 12 | (92) | 19 | (95) | 0.09⁺ |
| Peripheral Tfh | | 2.3 | (1.9-3.6) | 3.6 | (2.0-5) | |
| median (% in | | | | | | |
| CD4⁺) (IQR) | | | | | | |
| IgG anti-Esherichia | 0.55⁺ | 13 | (100) | 19 | (95) | 0.09⁺ |
| coli, n (%) | | | | | | |
| IgG anti-Esherichia | | 0.199 | (0.186-0.233) | 0.246 | (0.185-0.264) | |
| coli, median (SI) | | | | | | |
| (IQR) | | | | | | |
| IgG anti- | 0.05⁺ | 13 | (100) | 19 | (95) | 0.21⁺ |
| Staphylococcus | | | | | | |
| capitis, n (%) | | | | | | |
| IgG anti- | | 0.003 | (0.002-0.005) | 0.002 | (0-0.004) | |
| Staphylococcus | | | | | | |
| capitis, median | | | | | | |
| (SI) (IQR) | | | | | | |
| Translational research, dynamic between pre-treatment and cycle 1 | | | | | | |
| Circulating CXCL13, n (%) | 0.61⁺ | 8 | (62) | 14 | (70 | 0.01⁺ |
| Circulating CXCL13, | | 1.2 | (0.9-1.5) | 1.4 | (1.3-1.8) | |
| ratio (pre-/C1) (IQR) | | | | | | |
| Translational research, post-treatment | | | | | | |
| Intra-tumoral TLS, n (%) | 0.62⁺ | 13 | (100) | 19 | (95) | 0.21⁺ |
| Intra-tumoral TLS, | | 13.8 | (2.9-36.5) | 56 | (10.4-80.3) | |
| median (/mm²) | | | | | | |
| (IQR) | | | | | | |

Abbreviations: SI: Staining Index, TLS: Tertiary lymphoid structures Statistical analyses: Log-rank test^{#}, Mann-Whitney test⁺ and Chi-Square or Fisher's exact
tests.

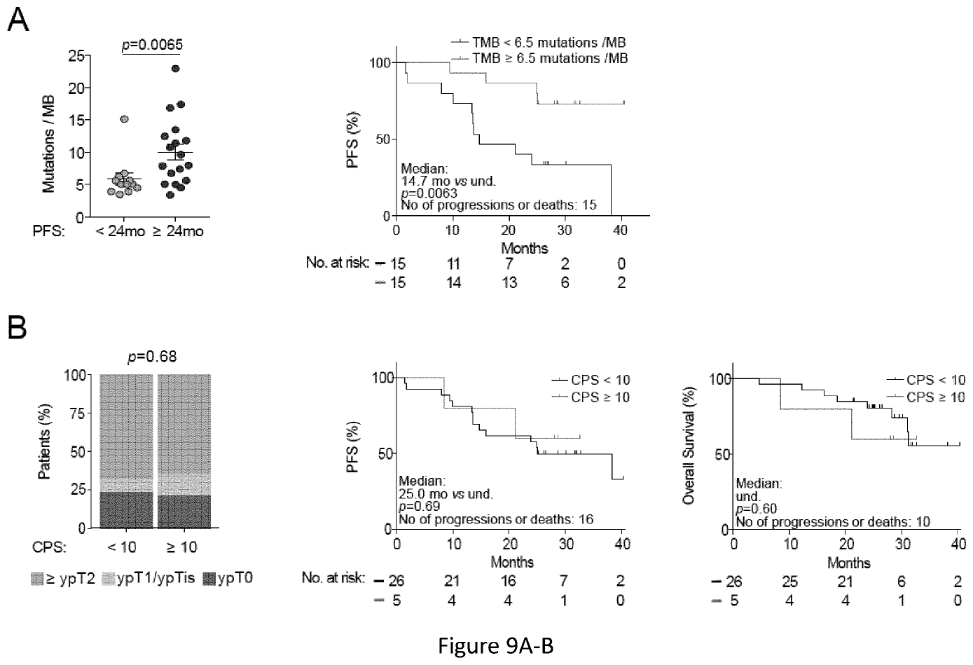

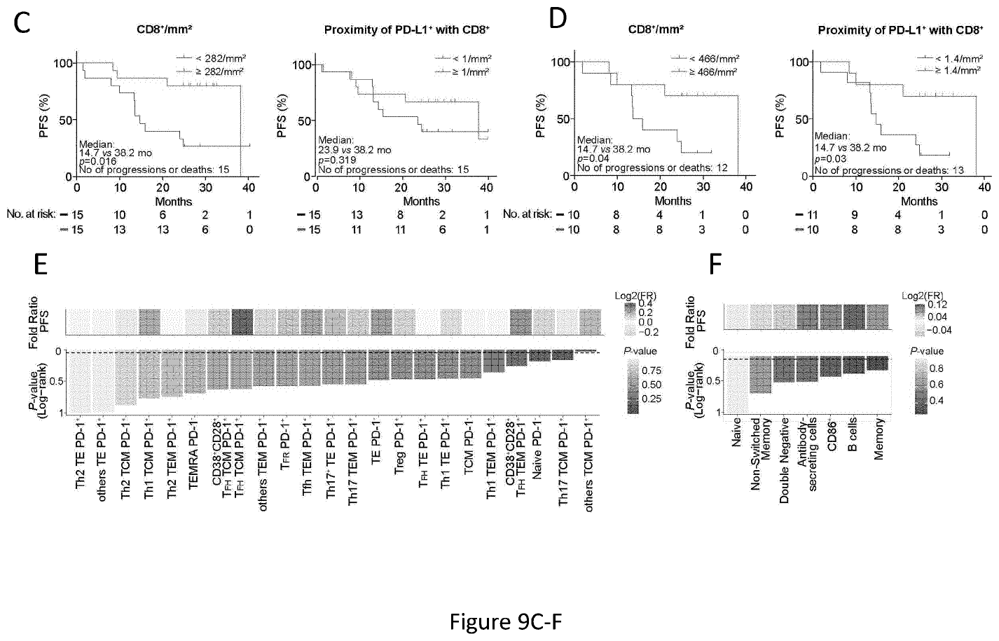

The median follow-up in efficacy population (n=34) was 28.7 months [min: 19.4; max: 40.5] at data cut-off. A relapse or a death occurred in 16 patients (FIG. 1 and FIG. 8C). The median progression-free survival (PFS) was 38.2 months (95% CI:15.9—not reached) (FIG. 1, left panel). With 10 deaths among all 34 patients, overall survival (OS) was 94.1% at 1 year and 78.9% at 2 years. Median OS was not reached (95% CI: 31—not reached) (FIG. 1, right panel). Somatic DNA sequencing concerned the 450 genes of the Cancer Core Europe panel (Eggermont et al., 2019). The most recurrent causal variants are Tumor Mutational Burden (TMB), genetic alterations in oncogenes and tumor suppressor genes or in the apoptotic machinery or in the DNA repair pathway (not shown). The co-occurrence of causal variants RB1 and TP53 (observed in n=8/30 MIBC) was associated with pCR and MPR (p=0.0042 and p=0.031, respectively, Fisher's exact test) but not with PFS (p=0.16, Log-rank test). As reported earlier (Necchi et al. 2018), high tumor mutational burden (TMB) was associated with prolonged PFS (FIG. 9A).

Hence, PANDORE study corroborated the efficacy of neoadjuvant ICBs in cisplatin-ineligible MIBC, as previously reported (Gao et al., 2020; Necchi et al., 2018; Powles et al., 2019; van Dijk et al., 2020) with 29.4% and 60.6% of patients who exhibited a pCR and did not relapse at 2 years post-TURBT, respectively. The efficacy population (n=34 patients, FIG. 8A) was analyzed henceforth for biomarker discovery according to PFS with a cut-off value of 24 months (n=13 with relapse and/or death occurring before 24 months and n=20 censored at 24 months, excluding one patient whose follow-up did not reach 24 months), as most relapses occur within the first 24 months in trials investigating chemotherapy and ICB (Bajorin et al., 2021; Pfister et al., 2021).

### Example 2: Baseline Follicular Helper T Cells (T_{FH}) are Associated with Clinical Benefit to Neoadjuvant Pembrolizumab

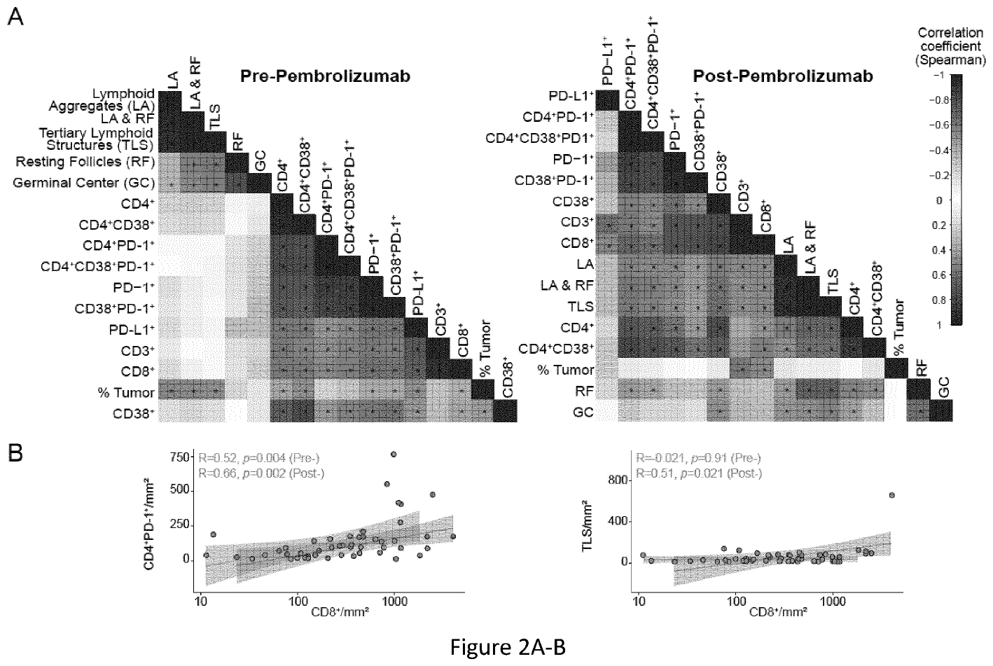

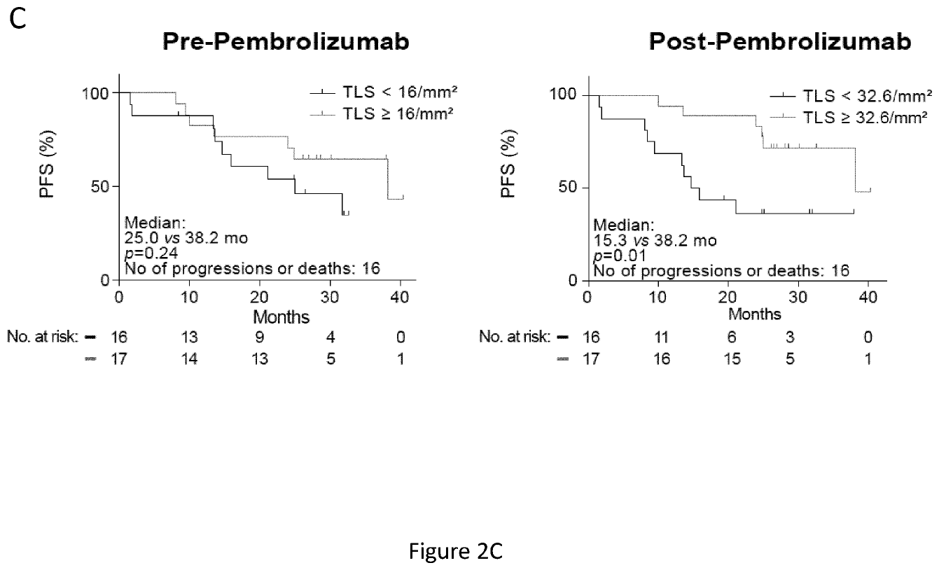

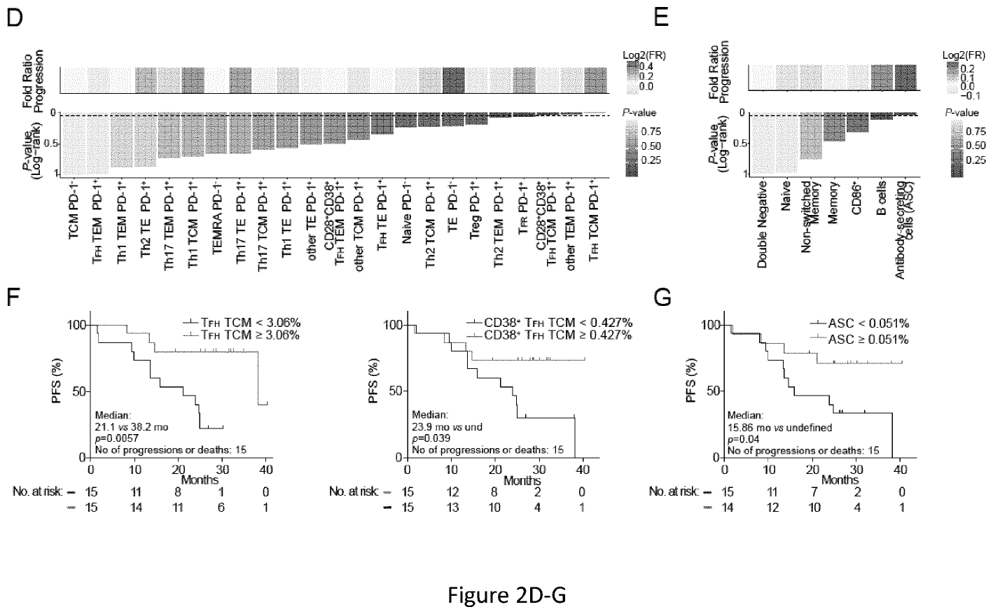

To determine which immune parameter best predict clinical benefit (MPR and PFS), we performed an exploratory biomarker analysis. We analyzed PD-L1 expression and tumor infiltrating lymphocytes (TILs) in tumor tissues obtained through TURBT (pre-pembrolizumab) and on cystectomy tissues (post-pembrolizumab) (FIGS. 9B, 9D). First, we performed immunohistochemistry staining using anti-PD-L1, -CD3, and -CD8 antibodies in matched pre- and post-treatment specimen in 34 patients. PD-L1 expression was analyzed in immune and tumor cells using the combined positive score (CPS, clone: PD-L1 22C3, FIG. 9B) on TURBT samples. Only 5 patients harbored a CPS >10 (16%), and CPS was not associated with pCR (p=0.68, Chi-square test) nor MPR (p=0.82, Chi-square test). CPS score failed to predict either PFS or OS (FIG. 9B). Density and geodistribution of CD3⁺ and CD8⁺ cells, as well as clustering and proximity of PD-L1⁺ and CD8⁺ cells (Immunoscore IC™) were evaluated (FIG. 2). The density of CD8⁺ cells, pre- and post-pembrolizumab, was associated with PFS (FIG. 9C-D). After PD-1 blockade, the proximity between CD8⁺ T and PD-L1⁺ cells was associated with prolonged PFS (FIG. 9D). Tertiary lymphoid structures like were identified by microscopic morphology in hematoxylin-eosin-safranin (HES)-stained tissues in association with CD20 and CD4, CD38, PD-1 immunolabeling as germinal centers (GC), resting follicles (RF) and lymphoid aggregates (LA). Together, these three structures defined the TLS-like. Spearman correlation matrices establishing interconnections between all these pathological factors (FIG. 2A-B). Thus, the density of CD4⁺PD-1⁺ cells correlated with CD8⁺ cells at baseline (FIG. 2A-B, left panel), and pembrolizumab triggered a coordinated association between TLS-like (and/or GC, RF, LA) and CD8⁺ cells (FIG. 2A-B, right panel). Moreover, the latter coincided with densities of CD4⁺PD-1⁺ (p=0.0022 and R=0.66, Spearman correlation) and anticorrelated with MPR (p=0.017 and R=−0.53, Spearman correlation) (FIG. 2A, right panel,). Finally, while baseline TLS-like did not predict PFS (p=0.24, Log-rank test), pembrolizumab-induced TLS-like were associated with PFS (p=0.01, Log-rank test) (FIG. 2C).

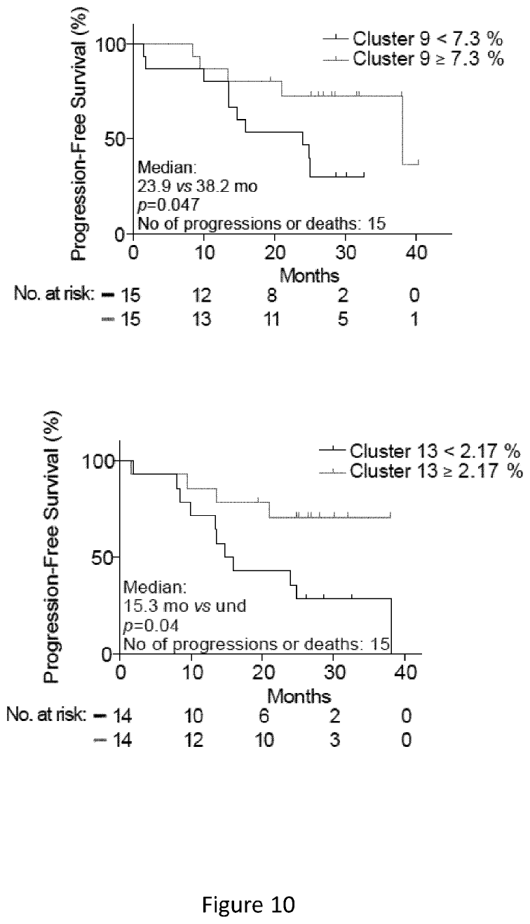

To deeply investigate the coordination between tumoral TLS-like and CD8 infiltrates with blood immune profiles induced by PD-1 blockade, we performed cytometry by time-of-flight (CyTOF) on paired blood cells before, during and after (cystectomy) 3 cycles of pembrolizumab in 38 patients. As mentioned above, the efficacy assessment was available for 34/38 patients. One hundred and fifty-three samples were barcoded (1 barcode for 1 timepoint per patient) and stained with a 38-parameter antibody panel for high-dimensional characterization of the CD4, CD8 and B cell heterogeneity based on canonical markers of polarization, differentiation and activation/exhaustion (Table 2). Using the PhenoGraph algorithm, we analyzed the clinical significance of different CD4⁺ T helper (TH) and regulatory T (Treg) subpopulations based on the relative expression of PD-1 and effector/memory markers for the response to pembrolizumab (Levine et al. 2015)) (not shown). The unsupervised PhenoGraph analysis of the PD-1-expressing clusters revealed that cluster 9 defined as CD4⁺PD-1⁺CD45RA⁻CD28⁺CD27⁺CD127⁺, which are central memory T cells (TCM) and cluster 13 or defined as CD4⁺PD-1⁺CD45RA⁻CD28⁺CD27⁺CD127^{low/-} cells, which are effector memory T cells (TEM), were the only circulating TH cells associated with PFS, at baseline (cluster 9 TCM) and after pembrolizumab (cluster 13 TEM) (FIG. 10). Focusing on specific fingerprints, we found that cluster 9 was selectively enriched for the canonical marker of T_{FH} (19% expressing CXCR5), while cluster 13 cells expressed the activation marker CD38 (46% CD38⁺, 9% CXCR5⁺), suggesting that pembrolizumab may preferentially target and activate T_{FH}. Hence, we performed a more biased analysis, applying a manual gating to investigate the clinical significance for PFS of all blood CD4⁺ T_{H} and B cells, pre- (FIGS. 2D, 2F and 9E) and post-pembrolizumab (FIGS. 2E, 2G and 9F). Central memory T_{FH}(CD4⁺CXCR5⁺PD-1⁺ TCM) and their activated fraction (CD38⁺CD28⁺CD4⁺CXCR5⁺PD-1⁺ TCM) stood out as the most significant circulating TH cells predicting PFS at baseline (FIG. 2D, 2F and FIG. 9E) while CD38^{high}Blimp1⁺CD19^{+/low} antibody-secreting cells (ASC) became associated with PFS only after pembrolizumab (FIG. 2E, 2G and FIG. 9F).

Altogether, neoadjuvant pembrolizumab mostly benefited MIBC patients who harbored pre-existing T_{FH} TCM cells which could orchestrate TLS-like formation and/or maturation as well as CD8⁺ T cell tumor infiltration.

TABLE 2
Pembrolizumab-related adverse events according to Common
Terminology Criteria for Adverse Events (CTCAE)

| | CTCAE Grade 1 (n = 16, 41%) | | CTCAE Grade 2 (n = 8, 21%) | |
|---|---|---|---|---|
| P# | System Organ Class | Preferred term | System Organ Class | Preferred term |
| 001 | | | Musculoskeletal and | Gouty arthritis |
| | | | connective tissue disorders | |
| 002 | Endocrine disorders | Hyperthyroidism | | |
| 003 | Skin and subcutaneous tissue disorders | Pruritus | | |
| 007 | Endocrine disorders | Hyperthyroidism | | |
| 008 | General disorders and administration site | Asthenia | | |
| | conditions | | | |
| 008 | Nervous system disorders | Paraesthesia | | |
| 010 | Gastrointestinal disorders | Diarrhoea | Gastrointestinal disorders | Diarrhoea |
| 010 | General disorders and administration site | Asthenia | Gastrointestinal disorders | Colitis |
| | conditions | | | microscopic |
| 011 | | | Eye disorders | Keratitis |
| 012 | Gastrointestinal disorders | Constipation | Endocrine disorders | Hypothyroidism |
| 015 | Vascular disorders | Hot flush | | |
| 020 | Skin and subcutaneous tissue disorders | Rash | | |
| 020 | General disorders and administration site | Asthenia | | |
| | conditions | | | |
| 020 | Gastrointestinal disorders | Diarrhoea | | |
| 021 | General disorders and administration site | Fatigue | | |
| | conditions | | | |
| 021 | Reproductive system and breast disorders | Penile erythema | | |
| 024 | Skin and subcutaneous tissue disorders | Pruritus | | |
| 024 | General disorders and administration site | Fatigue | | |
| | conditions | | | |
| 027 | Skin and subcutaneous tissue disorders | Psoriasis | Skin and subcutaneous | Psoriasis |
| | | | tissue disorders | |
| 027 | Endocrine disorders | Hyperthyroidism | | |
| 028 | General disorders and administration site | Fatigue | | |
| | conditions | | | |
| 028 | Endocrine disorders | Hypothyroidism | | |
| 029 | Musculoskeletal and connective tissue | Musculoskeletal | | |
| | disorders | pain | | |
| 029 | General disorders and administration site | Asthenia | | |
| | conditions | | | |
| 031 | Gastrointestinal disorders | Diarrhoea | | |
| 031 | Skin and subcutaneous tissue disorders | Pruritus | | |
| 035 | | | General disorders and | Asthenia |
| | | | administration site | |
| | | | conditions | |
| 037 | Endocrine disorders | Hypothyroidism | Endocrine disorders | Hypothyroidism |
| 038 | General disorders and administration site | Asthenia | | |
| | conditions | | | |
| 038 | Endocrine disorders | Hyperthyroidism | | |
| 039 | Gastrointestinal disorders | Nausea | | |
| 040 | Skin and subcutaneous tissue disorders | Pruritus | | |
| 043 | General disorders and administration site | Fatigue | | |
| | conditions | | | |
| 044 | Endocrine disorders | Hyperthyroidism | Endocrine disorders | Hypothyroidism |
| 045 | Endocrine disorders | Hyperthyroidism | | |

### Example 3: T_{FH} Accumulated within TLS-Like and Predicted Prolonged PFS

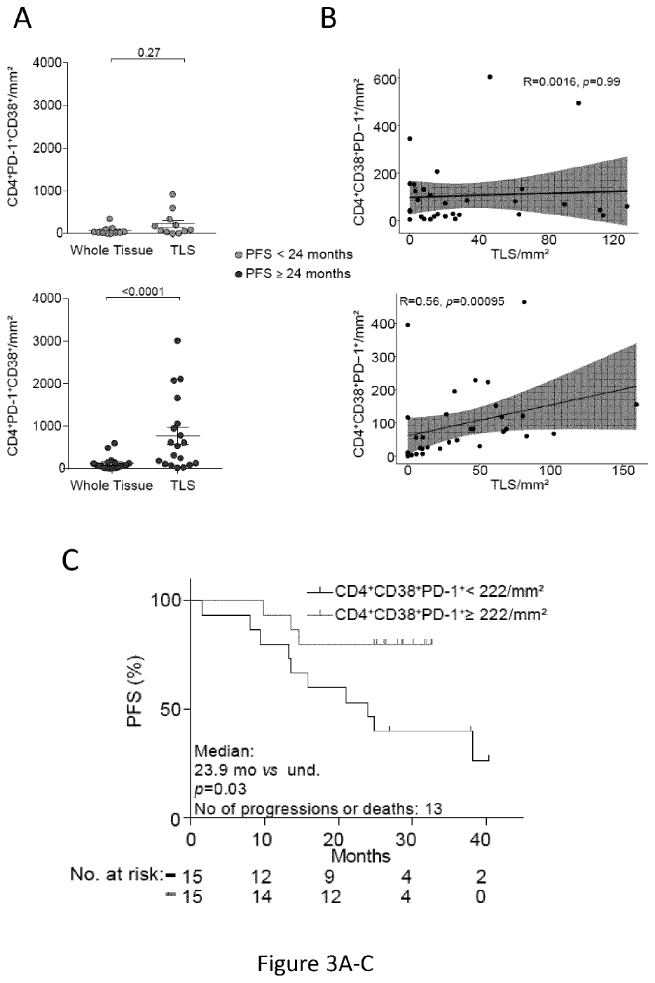

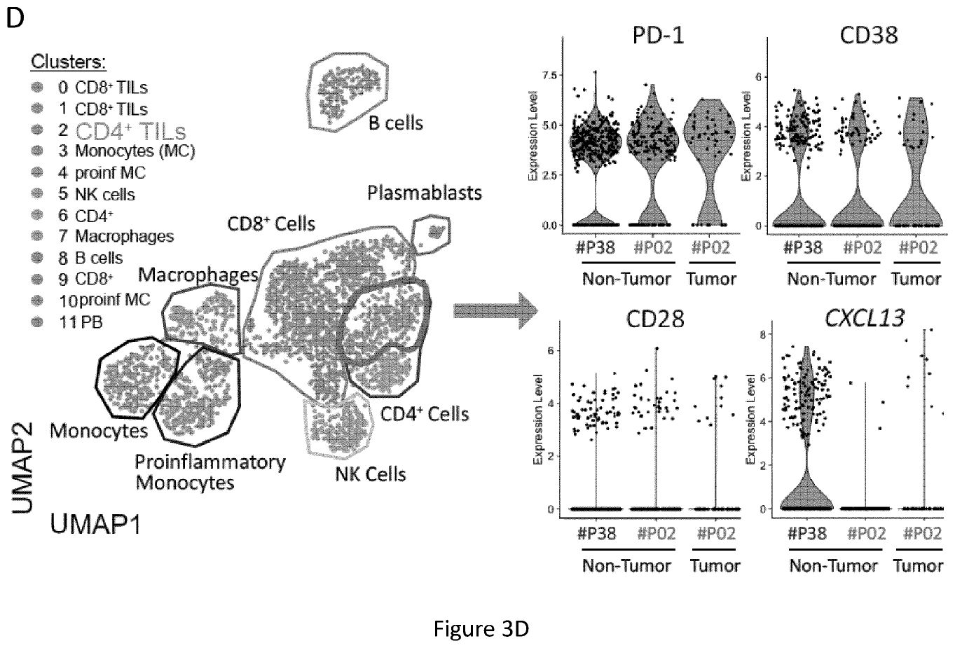

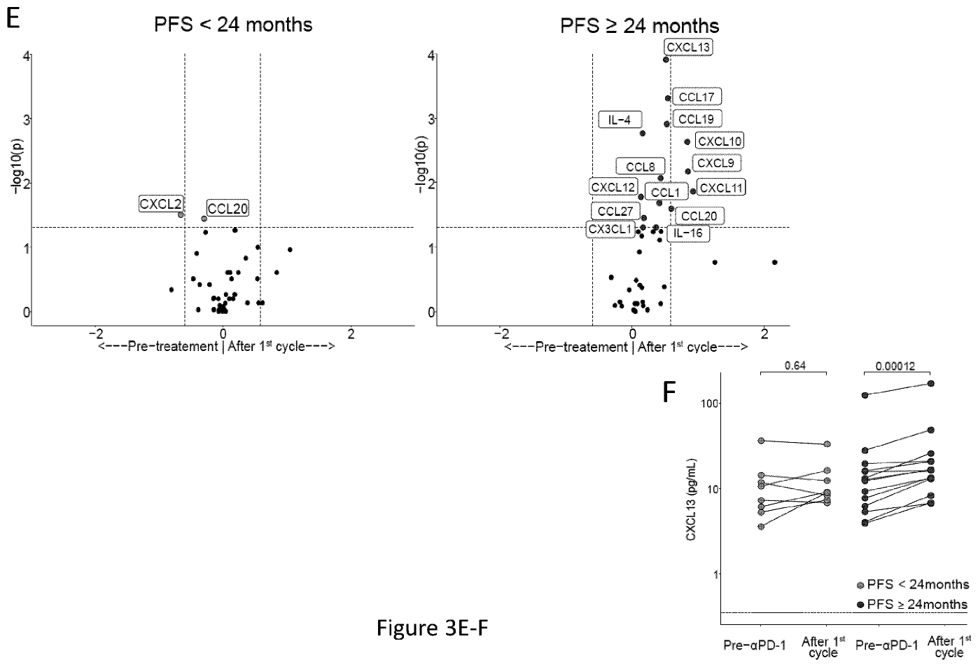

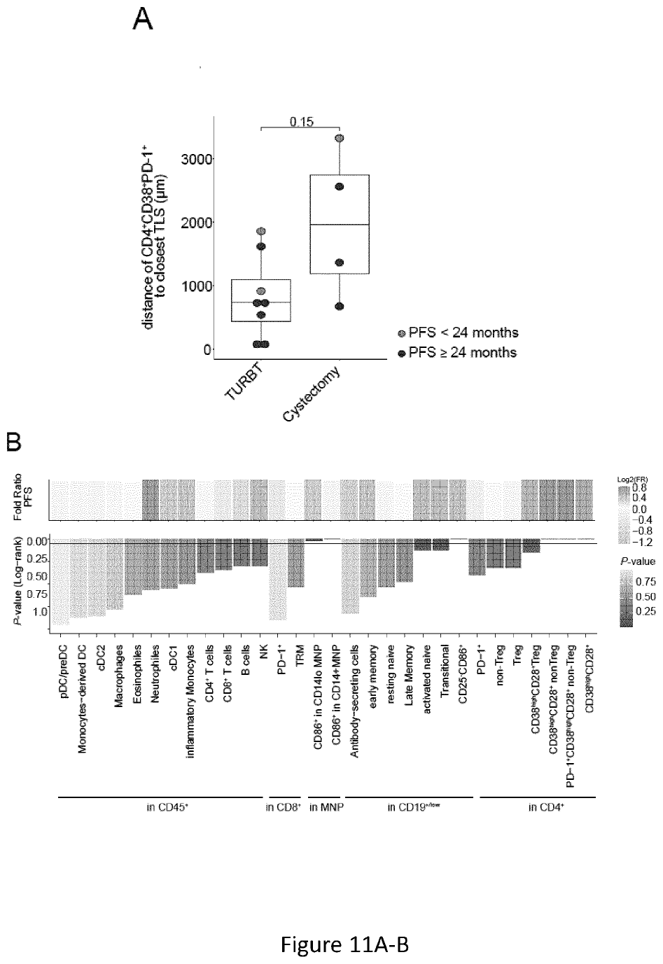

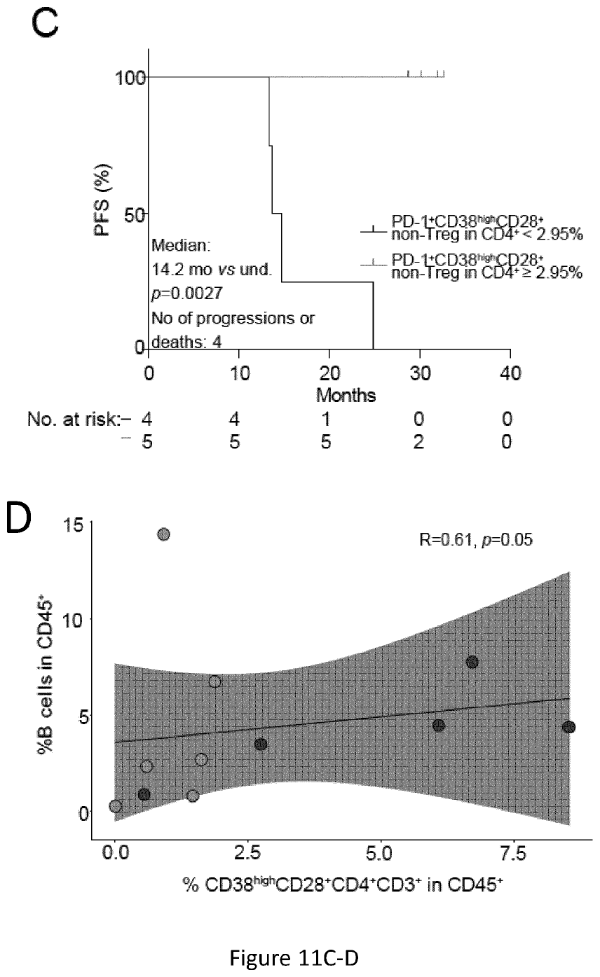

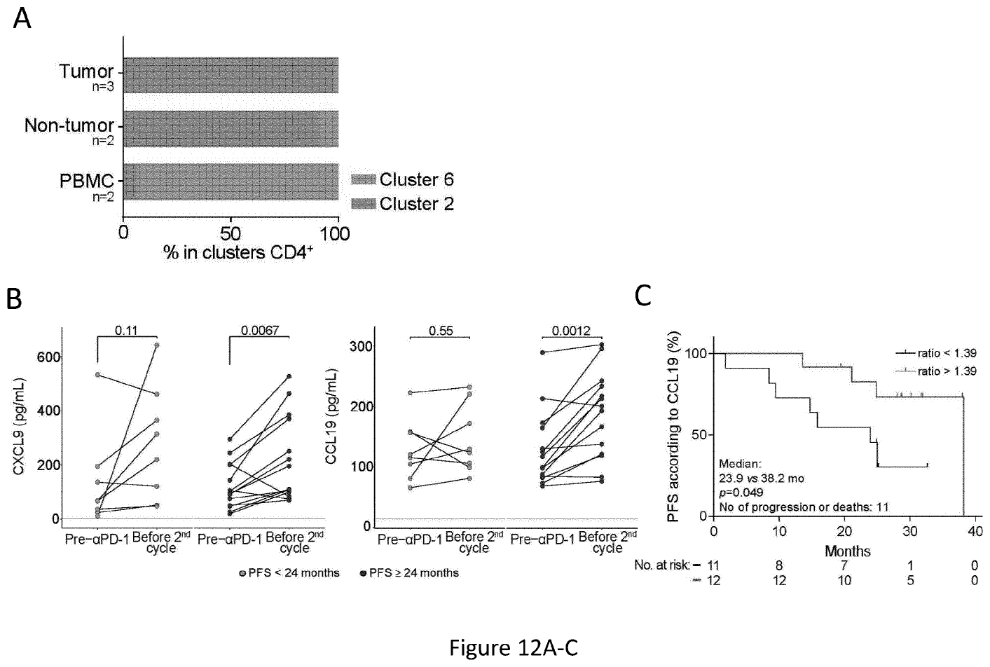

We next focused on the geodistribution of T_{FH} within TILs using immunofluorescence staining with mAbs specific for CD20, CD4, CD38, and PD-1 and -CD20 mAbs. When T_{FH} reach tumor beds, they downregulate their expression of CXCR5 (Gu-Trantien et al. 2017). To avoid this issue, we decided to not include CXCR5 in our tissue staining and to define T_{FH-like} as triple (CD4, CD38, PD-1) positive cells. Before pembrolizumab, the density of T_{FH-like} triple positive cells was enriched in the vicinity of TLS-like only in patients without progression at 24 months (FIG. 3A, lower panel). As shown with circulating T_{FH} TCM, the baseline density of intra-TLS T_{FH-like} triple positive cells was associated with prolonged PFS (FIG. 3C). Moreover, the density of such T_{FH-like} cells within the tissue correlated with TLS-like formation post- but not pre-pembrolizumab (FIG. 3B, lower vs upper panel). To further characterize T_{FH-like} cells within the tumor immune atlas post-pembrolizumab, we performed CyTOF in 11 available tumors post-cystectomy (Table 3). As observed in blood, the CD38^{high}CD28⁺ subset of non-regulatory CD4⁺ PD-1⁺ T cells best predicted PFS among all the TIL subsets (FIG. 11B-C) and correlated with B lymphocytes (FIG. 11D). In addition, we performed single cell RNA sequencing of blood CD45⁺ cells and tissue leukocytes from two tumor-free bladders, harvested from surgical specimens of a patient with pCR and PFS ≥24 months (P #38) and one MIBC from a non-responding patient with progressive disease (P #02) (Table 4). The unsupervised clustering of cell heterogeneity identified one cluster (cluster 2) enriched in CD4⁺ TILs (FIG. 3D and FIG. 12A). The CD38, CD28 and PD-1 specific oligo-conjugated antibodies allowed to measure the expression of the prototypical transcript of T_{FH} cells, CXCL13, only in the CD4⁺ TIL fraction of the patient exhibiting a pCR (FIG. 3D, right panel).

TABLE 3
Baseline characteristics for tumors analyzed by mass cytometry

| | Mass cytometry | | PFS < 24 months | | PFS ≥ 24 months | |
|---|---|---|---|---|---|---|
| | (n = 11) | | (n = 5) | | (n = 6) | |
| Age (years), median (IQR) | 76 | | 76 | | 73 | |
| | (65-79) | | (63-85) | | (65-79) | |
| Male sex, n (%) | 7 | (63.6) | 2 | (33.3) | 5 | (100) |

TNM stage, n (%)

| T2 | 8 | (72.7) | 5 | (100) | 3 | (50) |
|---|---|---|---|---|---|---|
| T3 | 0 | (0) | 0 | (0) | 0 | (0) |
| T4 | 2 | (18.2) | 0 | (0) | 2 | (33.3) |
| Not defined | 1 | (9.1) | 0 | (0) | 1 | (16.7) |
| Previous BCG, n (%) | 2 | (18.2) | 1 | (20) | 1 | (16.7) |
| PD-L1 positivity, n (%) | 3 | (27.3) | 1 | (20) | 2 | (33.3) |
| Median intratumoral | 120.9 | | 63.3 | | 364.6 | |
| CD8⁺ (cells/mm²) | | | | | | |

ECOG performance status, n (%)

| | | | | | | |
|---|---|---|---|---|---|---|
| 0 | 5 | (45.5) | 1 | (20) | 4 | (66.7) |
| 1 | 6 | (54.5) | 4 | (80) | 2 | (33.3) |

Pathological response

| | | | | | | |
|---|---|---|---|---|---|---|
| Major Pathological Response, | 2 | (18.2) | 0 | (0) | 2 | (33.3) |
| n (%) | | | | | | |

Progression-free survival

| | | | | | | |
|---|---|---|---|---|---|---|
| Death or relapse, n (%) | 6 | (54.5) | 5 | (100) | 1 | (16.7) |
| Median (IQR) | 24.8 | (13.4-30.2) | 13.4 | (5.1-14.2) | 29.4 | (27.7-32.1) |

Survival

| | | | | | | |
|---|---|---|---|---|---|---|
| Death, n (%) | 2 | (18.2) | 2 | (33.3) | 0 | (0) |
| Median (IQR) | 28.6 | (21.4-30.2) | 21.4 | (10.3-22.9) | 29.9 | (28.7-32.1) |

TABLE 4
Patient characteristics for tumor analyzed
by single cell RNA sequencing

| Sample | | |
|---|---|---|
| Tag | Tissue | Comment |
| 01 | Non-tumor, post-aPD-1 | PANDORE n°38, Complete |
| | | Responder to aPD-1, progression- |
| | | free |
| 02 | Non-tumor, post-aPD-1 | PANDORE n°02, Non Responder |
| | | to aPD-1, progression |
| 03 | Tumor, post-aPD-1 | PANDORE n°02, Non Responder |
| | | to aPD-1, progression |
| 04 | Tumor, untreated | B11, immuno-reactive to aPD-1 ex |
| | | vivo |
| 05 | Tumor, untreated | B12, non-immuno-reactive to aPD- |
| | | 1 ex vivo |
| 06 | PBMCs, post-aPD-1 | PANDORE n°38, Complete |
| | | Responder to aPD-1, progression- |
| | | free |
| 07 | PBMCs, post-aPD-1 | PANDORE n°02, Non Responder |
| | | to aPD-1, progression |

Finally, to determine which TH cell-related soluble hallmark could be induced by pembrolizumab, we monitored the dynamic circulating levels of 40 soluble immune and non-immune factors by bead-based multiplex assay prior to, during and after pembrolizumab in 38 patients. We found that CXCL13 stood out as the most significant chemokine systemically released in blood after the first cycle, and this early rise was associated with absence of progression at 24 months, as opposed to other Th1 markers (such as CXCL9) that tended to increase in all patients (FIG. 3E-F). Of note, CCL19, a homeostatic chemokine involved in the architectural organization of TLS (Jones, Hill, et Jones 2016; Sautès-Fridman et al. 2016), accompanied the rise of CXCL13 and was associated with prolonged PFS (FIG. 12B, right panel and 12C).

In conclusion, pembrolizumab facilitated the accumulation of T_{FH}-like cells in TLS and induced the release of the T_{FH}-prototypic CXCL13 chemokine in tissue lesions and the blood stream of patients who presented a long-term response to pembrolizumab.

### Example 4: MIBC Residing T_{FH} are Associated with Ex Vivo Responses to Pembrolizumab

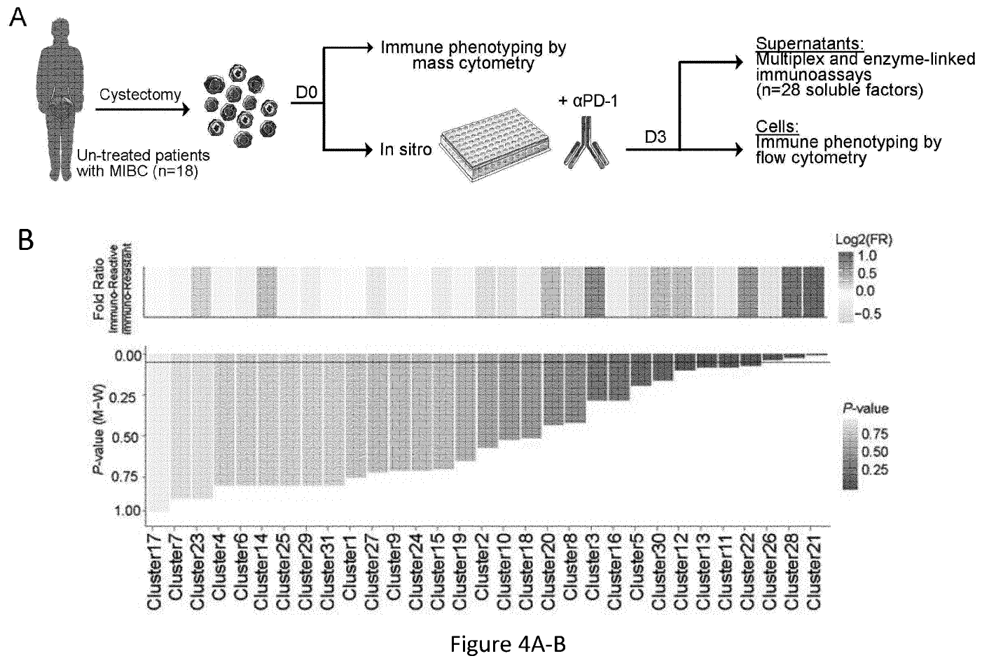

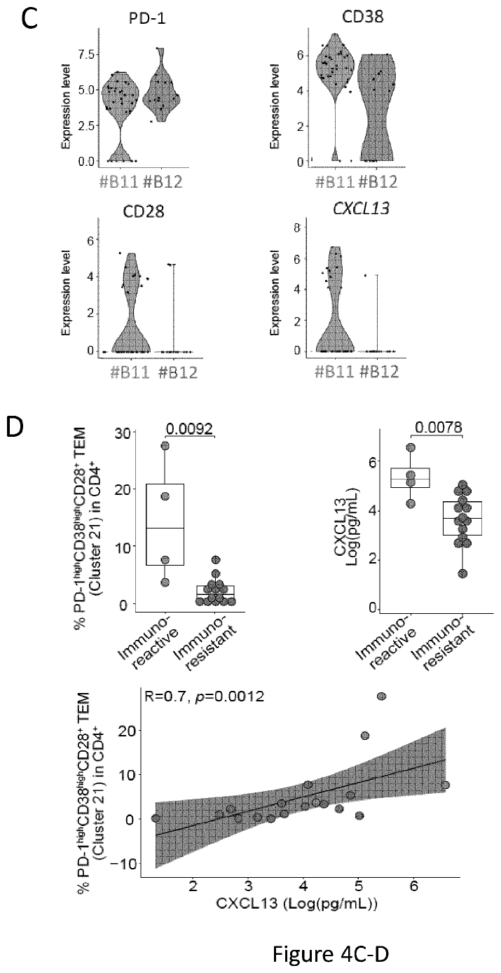

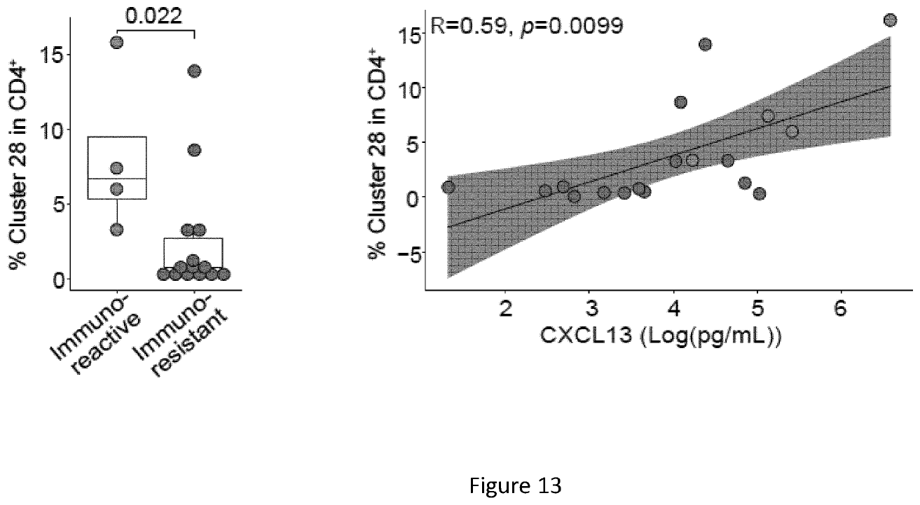

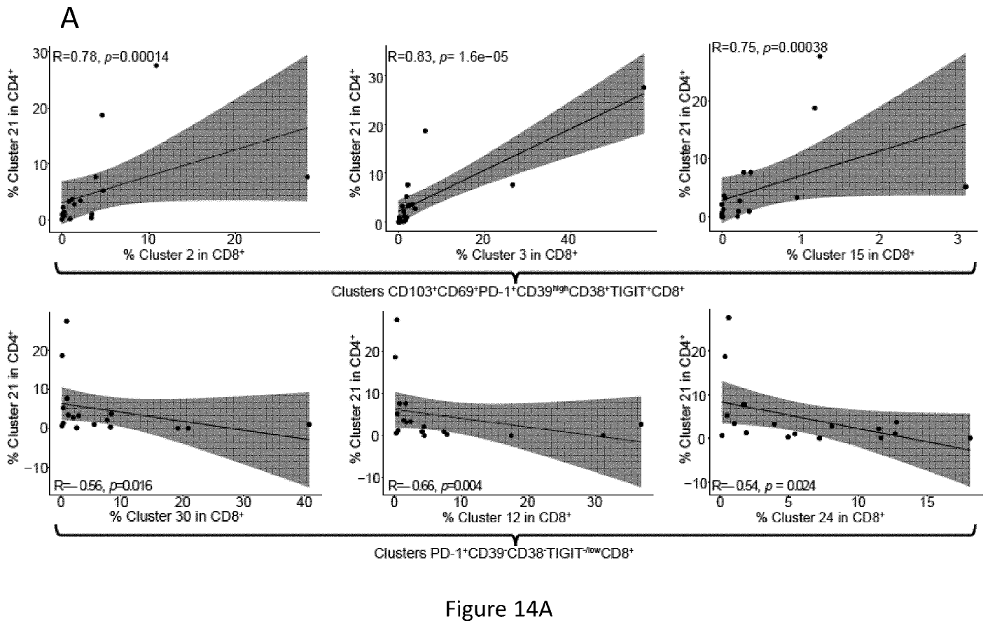

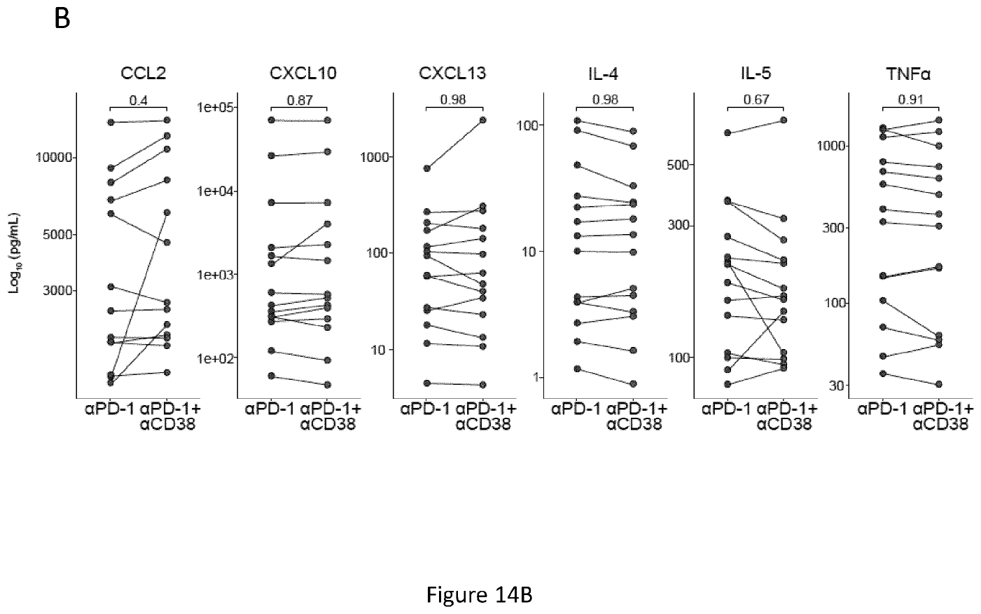

Next, we aimed at confirming the idea that the mere presence of T_{FH} cells within TILs is a prerequisite for the immunological response of MIBC to pembrolizumab. We took advantage of the previously reported dynamic system biology approach assessing immunoreactivity of fresh tumor pieces to immunomodulators including anti-PD-1 and anti-CD38 mAbs (Dubuisson et al., 2021; Jacquelot et al., 2017) (FIG. 4A). This in vitro/in situ (“in sitro”) assay has the potential to predict responses to anti-PD-1 mAbs in patients and may identify surrogate markers of immune reactivity to ICBs (Dubuisson et al. 2021; Voabil et al. 2021). Hence, we assessed the ex vivo immunoreactivity to pembrolizumab of eighteen surgical resections of MIBC specimens (56% pT3) from treatment naïve patients (Table 5, FIG. 4A). To calculate the index of immune stimulation by anti-PD-1 mAbs, we normalized raw values onto medium values, those being mostly equivalent to isotype control mAb-related values (Dubuisson et al. 2021). We used a non-supervised hierarchical clustering of z score-normalized concentrations of 28 immune and non-immune soluble factors monitored by bead-based multiplex assay and ELISA, as previously described (Dubuisson et al. 2021). The heatmap of this clustering highlighted two categories of MIBC patients, 22% (n=4/18) exhibiting increased levels of most analytes above the mean of the whole cohort after stimulation with anti-PD-1 mAbs (called henceforth “Immuno-reactive”, FIG. 4B) while 88% of MIBC patients failed to do so. The most significant differences between immuno-reactive and non-immuno-reactive tumors resided in the release of Th1/Th2 hallmarks (CXCL10, IFNγ, GM-CSF, CCL4, CCL5, Eotaxin, and IL-4) compatible with the T cell receptor cross-linking secretory profile (Dubuisson et al. 2021) as well as inflammatory cytokines (IL-1β, tumor necrosis factor TNFα, IL-6). We then examined correlates between in sitro immunoreactivity and basal CyTOF-based unsupervised clustering using PhenoGraph in these 18 MIBC. Tumor-infiltrating CD4⁺ TEM cells (CD45RA⁻CD127⁻CD28^{+/int}) harboring high expression levels of PD-1, CD38, and CD28 with co-expression of activation markers (CD69⁺CD95⁺CD39⁺) belonging to clusters 21 and 28 best predicted in sitro responsiveness to PD-1 blockade (FIG. 4B). We next performed single cell RNA sequencing on CD45⁺ from two independent MIBC-infiltrating CD45⁺ leukocytes populations at baseline (harvested from an immuno-reactive (B #11) and a non-immuno-reactive (B #12) tumor). Here again, within the cluster that mostly comprised CD4⁺ TILs (cluster 2, FIG. 12A), membrane expression of CD38, CD28 and PD-1 expression allowed to identify the T_{FH} pathognomonic transcript, CXCL13 only in the CD4⁺ TILs harvested from the immuno-reactive tumor (FIG. 4C), suggesting that CXCL13-T_{FH} which expressed PD-1 are critical to potentiate the induction of immune reactivity to ex vivo PD-1 blockade. Supporting this finding, the percentages of baseline CD4⁺CD38^{high}CD2⁺PD-1^{high} TILs (cluster 21 and 28) and spontaneous CXCL13 release correlated among each other and all were markedly higher in immune-reactive compared with non-immunoreactive MIBC (FIG. 40, FIG. 13). Of note, CXCL13-expressing CD8⁺ T cells were detectable but failed to segregate immunoreactive from non-immunoreactive tumors (not shown). In parallel, the unsupervised clustering of tumor-infiltrating CD8 (not shown) showed positive correlations between T_{FH} TILs (cluster 21) and CD38^{high}CD39^{high}PD-1^{high}TIGIT⁺CD69⁺CD103⁺ tissue-resident CD8⁺ TILs (TRM, FIG. 14A, top panels) but not with CD8⁺CD38⁻CD39⁻PD-1⁺ tissue-resident TILs or non-tissue resident CD8⁺ TLs (FIG. 14A, bottom panels). Although CD38 was used as a proxy marker to identify CXCL13-expressing PD-1⁺ T_{FH} CD4⁺ TIL subset, CD38 did not behave as a checkpoint inhibitory molecule, since we failed to increase or trigger TIL immunoreactivity by combining anti-CD38 with anti-PD-1 neutralizing mAbs in the in sitro system (FIG. 14B).

Altogether, PANDORE and in sitro studies both converged in showing that pembrolizumab potentiated TLS anti-tumor activity by coordinating T_{FH}, B cells and CD8⁺ TRM.

TABLE 5
Patient characteristics for tumors analyzed in in sitro assay

| | | | | | | Systemic | |
|---|---|---|---|---|---|---|---|
| Patient | | | | Stage | Stage | treatment | Ex vivo response |
| ID | Age | Sex | Histology | pT | pN | with aPD-1 | to aPD-1 |
| B5 | 51 | F | Epidermoid carcinoma | 3b | N0 | no | Non-Immuno-reactive |
| B6 | 63 | M | Urothelial carcinoma | 3a | N0 | no | Non-Immuno-reactive |
| B11 | 76 | M | Urothelial carcinoma | 3a | N0 | no | Immuno-reactive |
| B12 | 74 | F | Urothelial carcinoma | 2b | N0 | no | Non-Immuno-reactive |
| B13 | 53 | F | Urothelial carcinoma | 2b | N0 | no | Non-Immuno-reactive |
| B14 | 73 | M | Epidermoid carcinoma | 4a | N2 | no | Non-Immuno-reactive |
| B15 | 66 | M | Urothelial carcinoma | 1 | N0 | no | Non-Immuno-reactive |
| B16 | 72 | M | Urothelial carcinoma | 3b | N0 | no | Immuno-reactive |
| B19 | 80 | M | Urothelial carcinoma | 3b | N0 | no | Non-Immuno-reactive |
| B20 | 61 | M | Urothelial carcinoma | 3b | N0 | no | Non-Immuno-reactive |
| B21 | 44 | M | Urothelial carcinoma | 2a | N0 | no | Non-Immuno-reactive |
| B31 | 77 | M | Urothelial carcinoma | 4a | Nx | no | Non-Immuno-reactive |
| | | | (plasmacytoid) | | | | |
| B32 | 84 | M | Urothelial carcinoma | 4a | Nx | no | Immuno-reactive |
| B33 | 83 | M | Urothelial carcinoma | 4a | N0 | no | Non-Immuno-reactive |
| B34 | 70 | M | Urothelial carcinoma | 3b | N0 | no | Non-Immuno-reactive |
| B35 | 73 | M | Urothelial carcinoma | 3b | N2 | no | Non-Immuno-reactive |
| B36 | 70 | M | Urothelial carcinoma | 3 | N2 | no | Immuno-reactive |
| B38 | 67 | M | Urothelial carcinoma | 3b | N0 | no | Non-Immuno-reactive |

### Example 5: T_{FH} are Key Targets of Pembrolizumab Among CD4⁺ T Cells

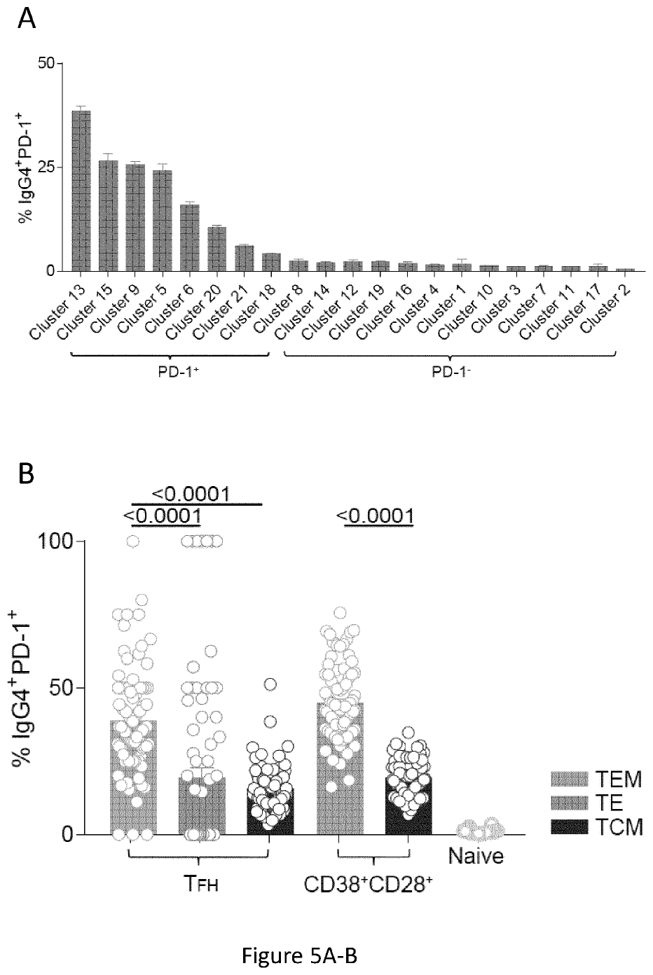

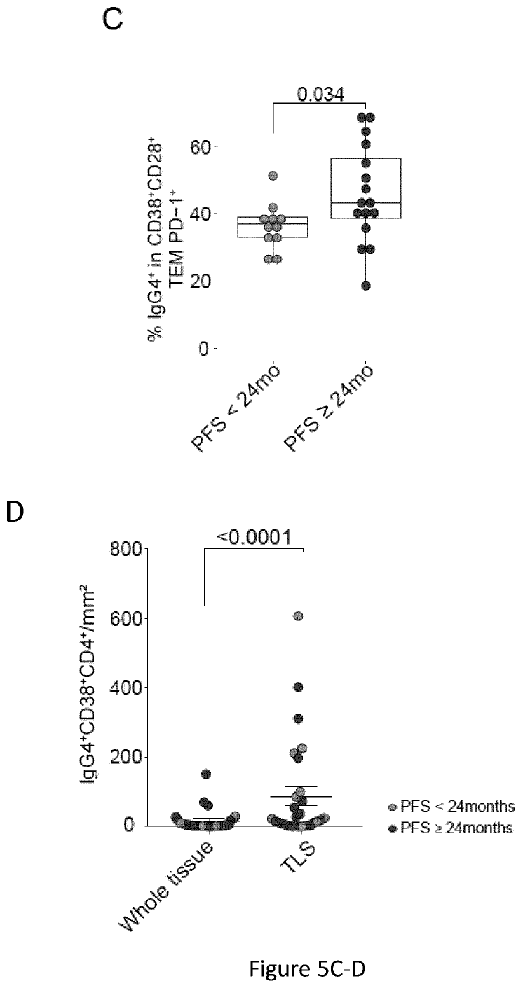

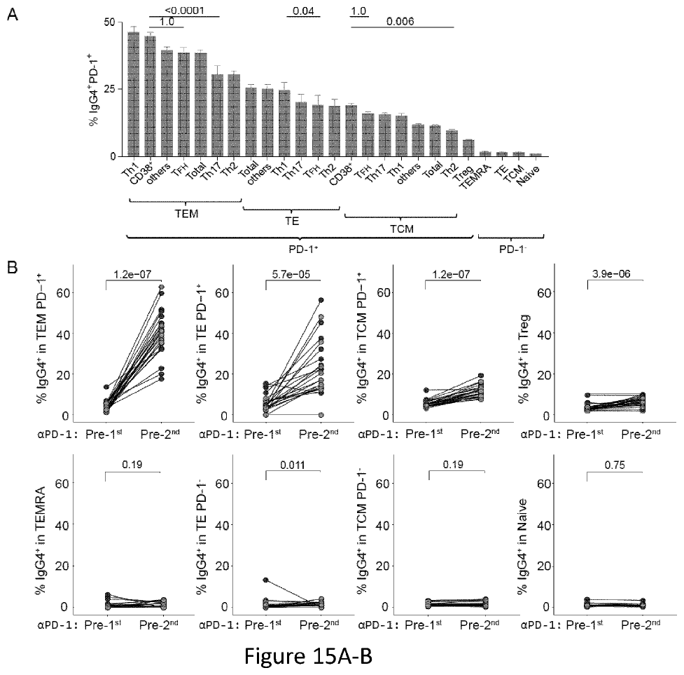

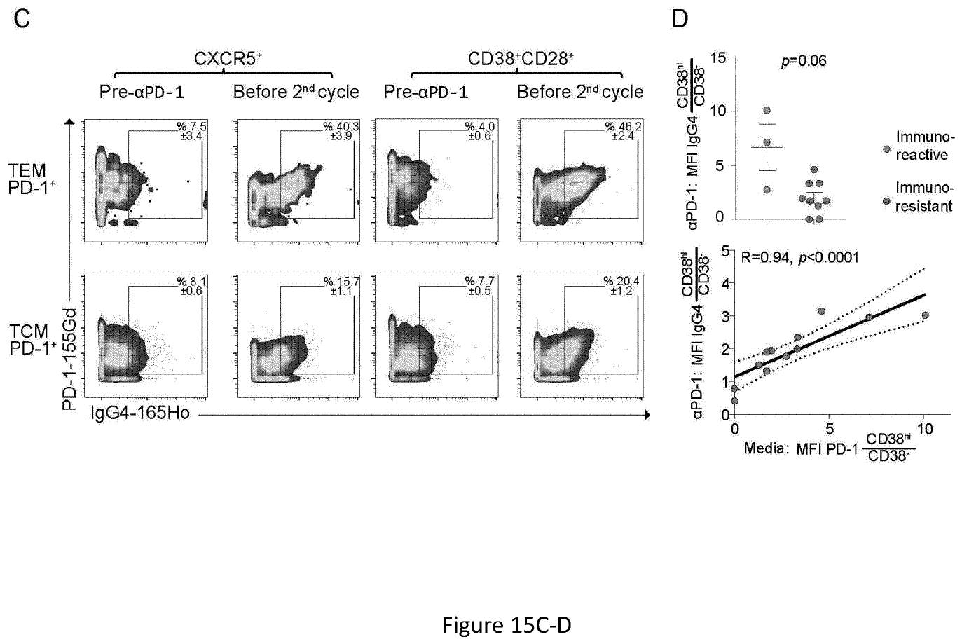

Visualizing the molecular targets of pembrolizumab IgG4 antibody is of the utmost interest for determining the mechanisms of action of this drug. Although many investigators identified CD8⁺ as the main functional targets of anti-PD1 mAbs (Huang et al. 2017; Ribas et al. 2016; Sade-Feldman et al. 2018), we took an unbiased approach to study the IgG4⁺ cells in blood and within cystectomy tissues, using CyTOF and immunofluorescence analyses. We confirmed that among blood CD4⁺PD-1⁺ cells, CD4⁺ TEM (cluster 13), as well as effector T cells (TE) (clusters 5+15) and to a lesser extent TCM (clusters 6+9+20) exhibited the strongest binding of anti-IgG4 mAbs during pembrolizumab treatment (FIG. 5A-B, FIG. 15A-C). Moreover, we detected a higher proportion of IgG4⁺ cells in the CD28⁺CD38⁺PD-1⁺ TEM T_{FH}-like subset (cluster 13), before the 3^{rd} injection of pembrolizumab in patients with a prolonged clinical benefit compared with patients with PFS<24 months (FIG. 5C). In tumor beds, IgG4 binding to CD38⁺ T_{FH-like} was particularly intense within tumor areas enriched in TLS-like (FIG. 5D).

After 3 days of in sitro stimulation with anti-PD-1 mAbs, the median fluorescence intensities (MFI) of IgG4 in CD38^{high} and CD38⁻ subsets revealed a trend for preferential binding to CD38^{high}-expressing CD4⁺ TILs in immuno-reactive tumors (FIG. 15D, top panel). As expected, there was a correlation between IgG4 and PD-1 positivity on this CD38^{high}CD4⁺ T cell subset post-pembrolizumab (FIG. 15D, lower panel).

Altogether, we show that pembrolizumab preferentially targeted circulating and tumor associated T_{FH} expressing CD38 and residing in TLS-like area.

### Example 6: E. coli-Specific Memory T_{FH} and B Cell Responses are Associated with Clinical Benefit to Neoadjuvant Pembrolizumab

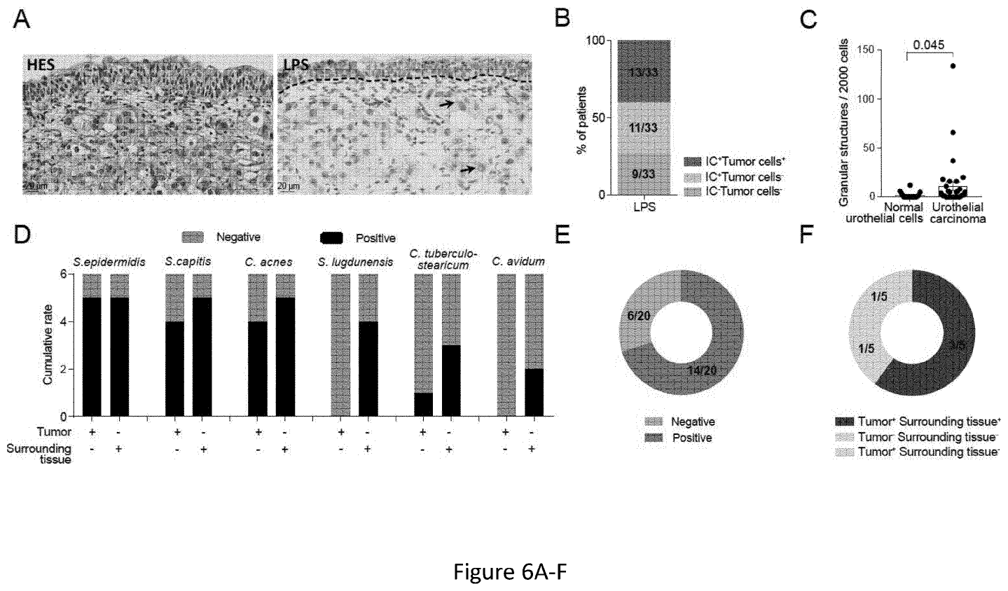

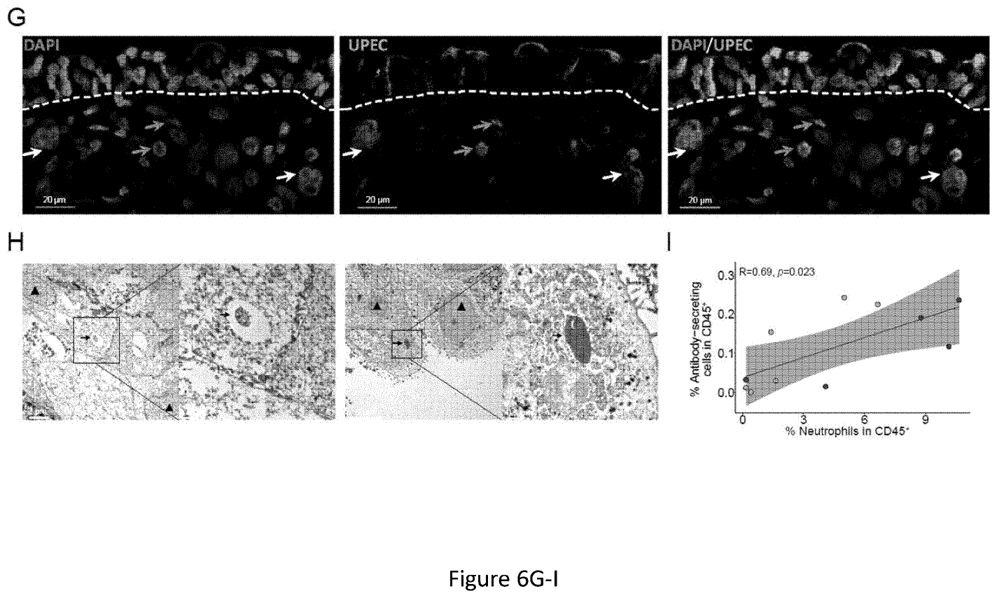

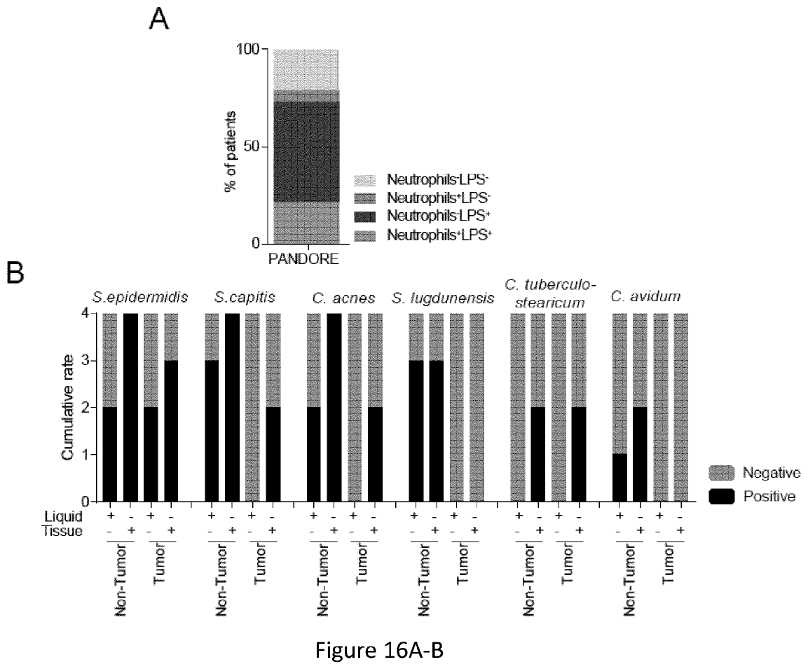

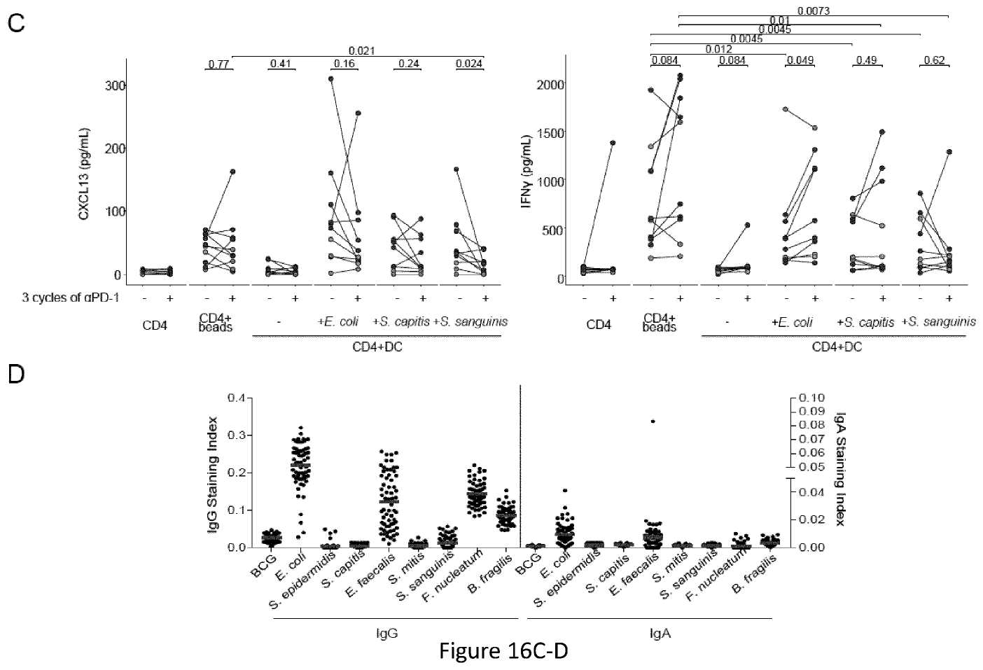

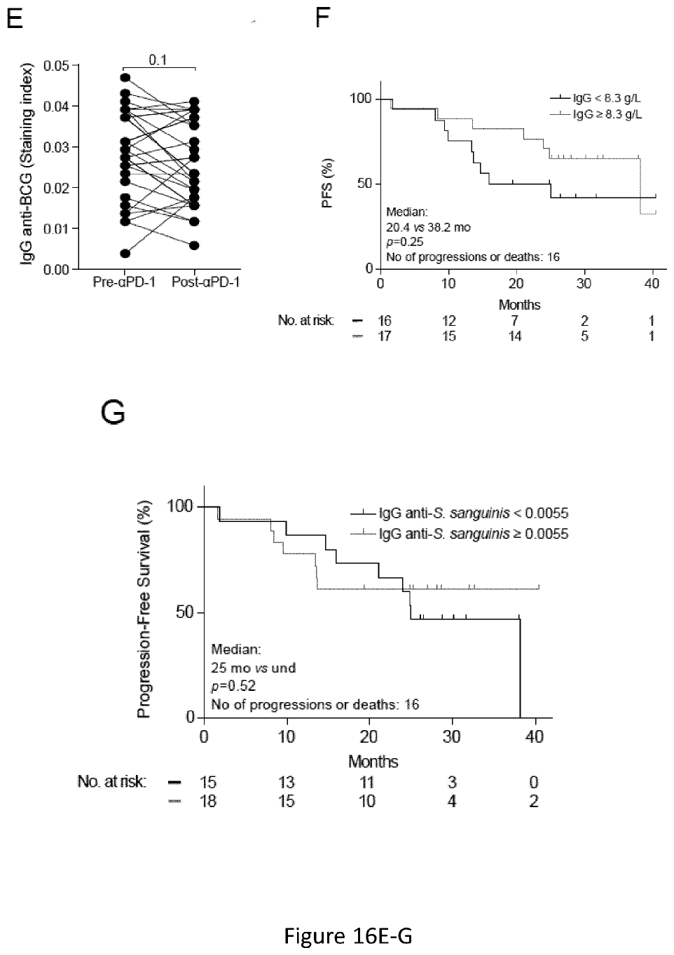

Urine is not sterile, and the commensal bacteria are present in the urinary tract from both healthy and UC patients (Dubourg et al. 2020; Brubaker et Wolfe 2016). Indeed, UC patients are commonly diagnosed with urinary tract infections (UTI) or asymptomatic bacteriuria (Vermeulen et al., 2015). Uropathogenic bacteria have the capacity to invade bladder epithelial cells and can persist for prolonged periods of time (Mulvey et al., 2001; Wu et al., 2017). Of note, the risk of developing UC in women is inversely correlated with urinary tract infections (Jiang et al., 2009). Additionally, patients diagnosed with non-invasive UC are treated by intravesical bacterial extracts (namely BCG: Bacillus Calmette-Guérin), which has been the case for 17.9% (n=7/39 patients) of patients enrolled in PANDORE study. Recent evidence indicates that the local and intratumoral microbiota influences prognosis of cancers developing at mucosal surfaces (Nejman et al., 2020; Pushalkar et al., 2018; Tsay et al., 2021). Therefore, we investigated the presence of tumor-associated bacteria in MIBC using immunohistochemistry (anti-LPS-staining), fluorescent in situ hybridization (FISH), electron microscopy, qPCR and culturomics, in aerobic and anaerobic conditions. LPS⁺ structures within UC cells and/or macrophages were observed in up to 70% of TURBT (n=24/33) (FIG. 6A-B), coinciding with cytoplasmic granular structures and exceptionally neutrophils (FIG. 16A). These granular structures were more numerous in tumor beds than in normal urothelial cells from surrounding tumor-free tissues and might correspond, at least partially, to Gram⁺ bacteria that we isolated in most of the fresh MIBC that we cultivated (n=6) (FIG. 6C-D). In particular, culturomics of primary bladder tumors and surrounding non-cancerous tissues allowed us to identify Staphylococcus capitis in the tumor tissues, but not in the transport supernatant of the tumor, suggesting that S. capitis was not a contamination (FIG. 6D, FIG. 16B). Given the ability of Escherichia coli to invade urothelial cells, we ran qPCRs using E. coli-specific probe sets (Mulvey, Schilling, et Hultgren 2001; Wu, Miao, et Abraham 2017) and could detect specific signal in 70% of the cases (n=14/20 of total cases with n=7/11 untreated specimen and n=7/9 post-pembrolizumab), without differences between tumors and surrounding “healthy” tissues (FIG. 6E-F). FISH using specific probes (chuA, c3686, c3509) (Brons et al. 2020) allowed to visualize uropathogenic E. coli (UPEC) in tumor and myeloid cells (FIG. 6G). Finally, we confirmed the presence of live bacilli within bladder tissues by transmission electron microscopy (FIG. 6H). Of note, we found a strong correlation between neutrophils and ASC within tumor infiltrating CD45⁺ cells post-pembrolizumab (FIG. 6I), suggesting that innate and humoral immune responses against bacteria might cooperate in the tumor beds.

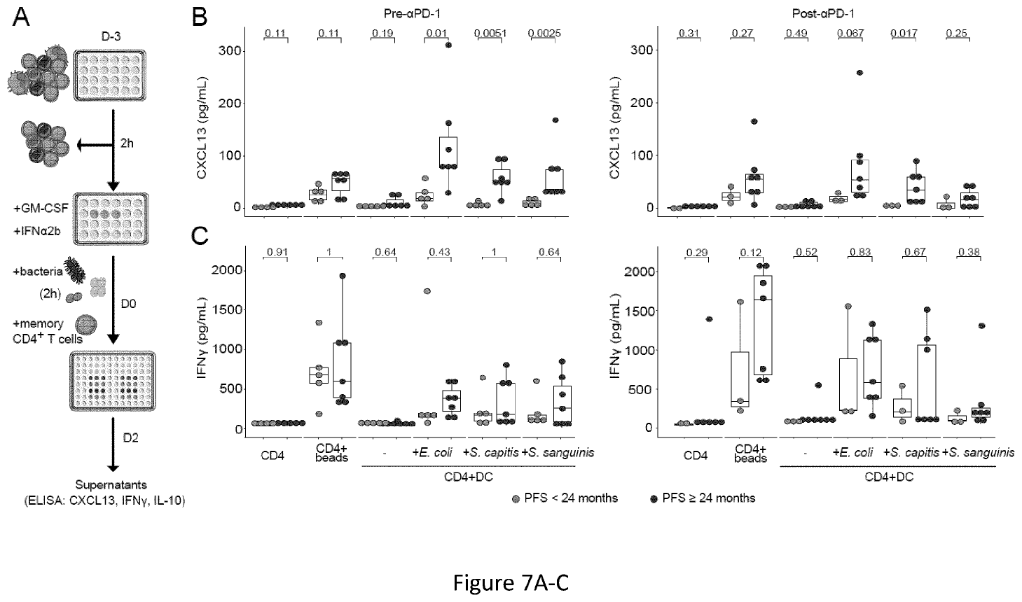

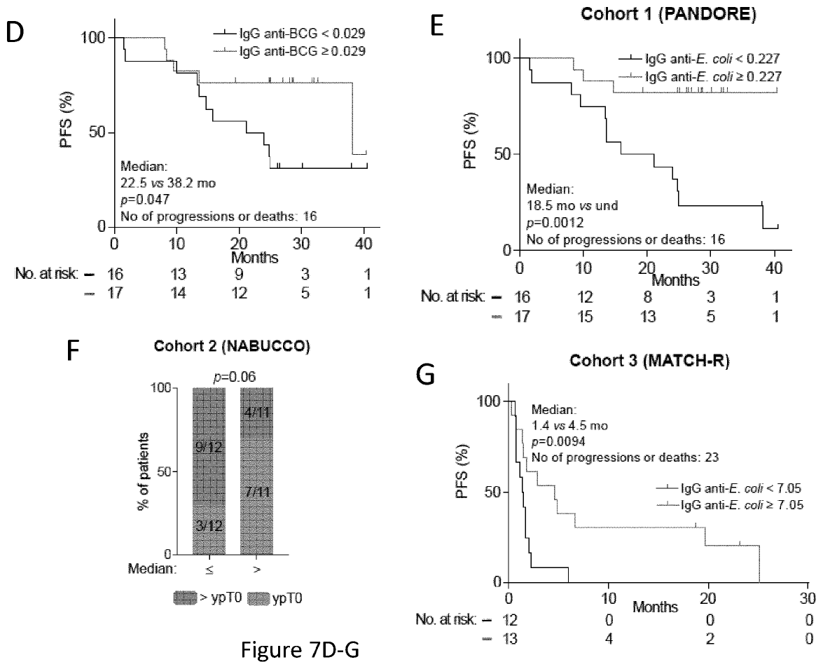

Hence, we addressed whether memory T or B cell immune responses directed against E. coli, BCG and other urinary bacterial species could be detected pre- and post-pembrolizumab. We first performed a longitudinal study of blood CD4⁺ T cell recall responses against urinary tract and tumor bacteria, monitoring cytokines prototypic of Th1 (IFNγ), T_{FH}(CXCL13) and regulatory (IL-10) T cell responses (Daillère et al. 2016; Routy et al. 2018; Vétizou et al. 2015) (Table 6 and FIG. 7A). UC patients harbored pre-existing commensal-specific Th1 and T_{FH} memory T cell responses that tended to increase and decrease by pembrolizumab for the former and the latter, respectively (FIG. 7B-C, FIG. 16C). Interestingly, CXCL13-producing T_{FH} but not IFNγ releasing TH1 memory T cell responses against E. coli and S. capitis were associated with the absence of progression at 24 months (FIG. 7B-C).

TABLE 6
Dynamics of humoral and cellular immune responses against commensals

| Pre- | Humoral | PFS < 24 mo (n = 13) | | PFS > 24 mo (n = 19) | | |
|---|---|---|---|---|---|---|
| treatement | Cellular | PFS < 24 mo (n = 5) | | PFS > 24 mo (n = 7) | | |
| Bacteria | Measurement | Mean | SEM | Mean | SEM | P-value |
| | IgA | 1.983 | 0.32 | 2.818 | 0.369 | 0.045 |
| | IgG | 8.285 | 0.718 | 8.703 | 0.333 | 0.32 |
| BCG | IgA | 0.0008 | 0.0001 | 0.00086 | 0.00005 | 0.59 |
| | IgG | 0.024 | 0.003 | 0.03074 | 0.0022 | 0.11 |
| E. coli | IgA | 0.0075 | 0.0017 | 0.0085 | 0.0015 | 0.6 |
| | IgG | 0.196 | 0.016 | 0.228 | 0.014 | 0.09 |
| | IFNγ | 463.2 | 316.5 | 367.5 | 71.24 | 0.43 |
| | CXCL13 | 23.86 | 9.083 | 120.7 | 35.07 | 0.01 |
| | IL-10 | 119 | 24.36 | 246.5 | 24.17 | 0.012 |
| S. epidermidis | IgA | 0.002 | 0.0003 | 0.0015 | 0.00022 | 0.12 |
| | IgG | 0.0014 | 0.0005 | 0.0035 | 0.0023 | 0.98 |
| S. capitis | IgA | 0.0022 | 0.0002 | 0.0016 | 0.00014 | 0.014 |
| | IgG | 0.0044 | 0.001 | 0.003 | 0.00094 | 0.21 |
| | IFNγ | 236.8 | 101.8 | 340.9 | 115 | 1 |
| | CXCL13 | 5.774 | 2.255 | 57.02 | 10.65 | 0.0051 |
| | IL-10 | 40.7 | 9.889 | 108.5 | 10.42 | 0.0061 |
| E. faecalis | IgA | 0.011 | 0.006 | 0.006 | 0.0011 | 0.8 |
| | IgG | 0.1185 | 0.02 | 0.1183 | 0.017 | 0.86 |
| S. mitis | IgA | 0.0016 | 0.0002 | 0.0016 | 0.00014 | 0.97 |
| | IgG | 0.0024 | 0.001 | 0.0056 | 0.0015 | 0.23 |
| S. sanguinis | IgA | 0.00085 | 0.00005 | 0.0016 | 0.0002 | 0.0095 |
| | IgG | 0.0088 | 0.003 | 0.0167 | 0.004 | 0.31 |
| | IFNγ | 211.9 | 97.22 | 347.1 | 119.2 | 0.64 |
| | CXCL13 | 8.856 | 3.279 | 63.99 | 18.63 | 0.0025 |
| | IL-10 | 42.15 | 11.7 | 124.9 | 16.64 | 0.042 |
| F. nucleatum | IgA | 0.0011 | 0.0006 | 0.0012 | 0.0007 | 0.74 |
| | IgG | 0.1351 | 0.008 | 0.148 | 0.007 | 0.47 |
| B. fragilis | IgA | 0.0023 | 0.00029 | 0.004 | 0.0005 | 0.031 |
| | IgG | 0.088 | 0.0049 | 0.089 | 0.006 | 0.63 |

| Post- | Humoral | PFS < 24 mo (n = 13) | | PFS > 24 mo (n = 18) | | |
|---|---|---|---|---|---|---|
| treatment | Cellular | PFS < 24 mo (n = 3) | | PFS > 24 mo (n = 7) | | |
| Bacteria | Measurement | Mean | SEM | Mean | SEM | P-value |
| | IgA | 1.958 | 0.30 | 2.626 | 0.39 | 0.21 |
| | IgG | 8.512 | 0.73 | 9.24 | 0.41 | 0.28 |
| BCG | IgA | 0.0007 | 0.00005 | 0.001 | 0.0001 | 0.031 |
| | IgG | 0.022 | 0.003 | 0.028 | 0.002 | 0.074 |
| E. coli | IgA | 0.0131 | 0.003 | 0.008 | 0.001 | 0.29 |
| | IgG | 0.205 | 0.017 | 0.232 | 0.01 | 0.13 |
| | IFNγ | 653.4 | 439.5 | 713.6 | 172.3 | 0.832 |
| | CXCL13 | 17.61 | 5.512 | 82.26 | 30.98 | 0.067 |
| | IL-10 | 117.7 | 8.609 | 195.2 | 21.61 | 0.067 |
| S. epidermidis | IgA | 0.0027 | 0.0026 | 0.0015 | 0.0002 | 0.0037 |
| | IgG | 0.0012 | 0.0006 | 0.0017 | 0.00142 | 0.38 |
| S. capitis | IgA | 0.0024 | 0.00017 | 0.00181 | 0.00016 | 0.032 |
| | IgG | 0.00513 | 0.0011 | 0.0031 | 0.00092 | 0.047 |
| | IFNγ | 264.5 | 134.5 | 567.2 | 229.9 | 0.67 |
| | CXCL13 | 4.411 | 2.579 | 39.31 | 11.56 | 0.017 |
| | IL-10 | 40.27 | 4.957 | 65.64 | 16.37 | 0.38 |
| E. faecalis | IgA | 0.004746 | 0.0012 | 0.00733 | 0.0012 | 0.14 |
| | IgG | 0.1121 | 0.0174 | 0.131 | 0.019 | 0.6 |
| S. mitis | IgA | 0.0018 | 0.0001 | 0.00157 | 0.0001 | 0.28 |
| | IgG | 0.0017 | 0.0007 | 0.0056 | 0.0016 | 0.16 |
| S. sanguinis | IgA | 0.0011 | 0.0002 | 0.00156 | 0.0002 | 0.21 |
| | IgG | 0.0043 | 0.0015 | 0.0145 | 0.004 | 0.18 |
| | IFNγ | 123.9 | 41.54 | 323 | 162.5 | 0.38 |
| | CXCL 13 | 6.719 | 6.719 | 18.21 | 6.182 | 0.25 |
| | IL-10 | 28.72 | 11.67 | 45.49 | 13.48 | 0.52 |
| F. nucleatum | IgA | 0 | | 0.0009 | 0.0005 | |
| | IgG | 0.1317 | 0.0072 | 0.148 | 0.0087 | 0.14 |
| B. fragilis | IgA | 0.002 | 0.0004 | 0.0038 | 0.0006 | 0.023 |
| | IgG | 0.0.87 | 0.005 | 0.0859 | 0.005 | 0.66 |

P-values were calculated using unpaired t test (Mann-Withney)

Next, we investigated the humoral immune responses against urinary tract and tumor commensals (Table 6). Very high serum titers of IgG and IgA antibodies were found against commensals shared between urinary and digestive tracts (such as E. coli or Enterococcus faecalis, FIG. 16D) contrasting with humoral responses toward skin commensals. Since BCG had been instilled into the bladder of 18% of our patients with a history of non-muscle-invasive UC and given that most patients were vaccinated with BCG, we also found detectable titers of IgG against BCG that tended to decrease with neoadjuvant pembrolizumab (FIG. 16E). In fact, preexisting BCG-specific IgG titers were associated with PFS (FIG. 7D). In line with the clinical significance of E. coli-specific memory T_{FH} responses, baseline E. coli-specific IgG (but not IgA) titers best predicted PFS in the PANDORE study (FIG. 7E, Table 6). In contrast, total IgG levels or IgG specific for other commensals did not have any prognostic value (FIG. 16F-G). In line with the frequent isolation of S. capitis from fresh MIBC, we found a potential clinical relevance for S. capitis-specific IgA responses (pre- and post-pembrolizumab) (Table 6). Supporting the findings obtained on the PANDORE cohort, we confirmed the putative predictive value of baseline anti-E. coli IgG for pCR in an independent cohort of 23 patients treated with neoadjuvant ipilimumab and nivolumab in the NABUCCO clinical trial (van Dijk et al. 2020) (FIG. 7F). In a third independent cohort of 25 metastatic MIBC patients treated with pembrolizumab in the MATCH-R trial (Table 7), we again validated baseline anti-E. coli IgG as a putative biomarker of PFS (FIG. 7G).

We conclude that MIBC contain immunogenic E. coli pathobionts eliciting T_{FH} and B cell responses that predict the clinical outcome of anti-PD-1-based immunotherapy.

TABLE 7
Baseline characteristics of patients from the validation cohort 3

| | MATCH-R | |
|---|---|---|
| Age (years), median (IQR) | 71 | |
| | (63.5-76) | |
| Male sex, n (%) | 18 | (72) |

ECOG performance status, n (%)

| | | |
|---|---|---|
| 0 | 6 | (24) |
| 1 | 19 | (76) |

Response (RECIST), n (%)

| | | |
|---|---|---|
| PR + CR | 4 | (16) |
| SD + PD | 21 | (84) |

Progression-free survival

| | | |
|---|---|---|
| Death or relapse, n (%) | 23 | (92) |

| | |
|---|---|
| Median | 1.7 |
| (IQR) | (0.9-5.4) |

Survival

| | | |
|---|---|---|
| Death, n (%) | 6 | (24) |

| | |
|---|---|
| Median | 5.3 |
| (IQR) | (2.1-11.5) |

### Discussion

In reported trials highlighting the efficacy of neoadjuvant ICBs in UC, immune or tumor expression of PD-L1 and TMB did not represent reliable baseline predictors of clinical benefit (Gao et al., 2020; Necchi et al., 2018; Powles et al., 2019; van Dijk et al., 2020). In fact, responding tumors showed predominant expression of genes related to tissue repair while resistance was associated with stromal or cell cycle fingerprints after neoadjuvant PD-L1 mAbs (Powles et al., 2019). This study also showed pre-existing intraepithelial CD8⁺ T cells and a pro-Th1 signature as predictive biomarkers of pCR to PD-1/PD-L1 blockade. In accordance with previously described biomarkers in melanoma and sarcoma (Cabrita et al., 2020; Helmink et al., 2020; Petitprez et al., 2020), TLS represented the best hallmark of response to neoadjuvant PD-1/PD-L1 plus CTLA-4 blockade in high grade MIBC (Gao et al., 2020; van Dijk et al., 2020). Indeed, TLS identified by immunohistochemistry were associated with a transcriptome blueprint centered around POU2AF1, LAMP3, CD79A and MS4A1, predicting pathological responses (Gao et al., 2020). However, these reports did not identify blood immune proxies associated with the induction of TLS in bladder tissues (Gao et al., 2020; van Dijk et al., 2020). Here, we showed that blood and tumor T_{FH} represent prominent targets for neoadjuvant pembrolizumab and a critical link with TLS where they preferentially reside and tumor infiltration by CD8⁺ T cells culminating in clinical benefit. We used several complementary approaches (immunohistochemistry, mass cytometry, functional in sitro assay) to show that pembrolizumab preferentially binds to effector T_{FH}—that express higher densities of PD-1 molecules than other TH subsets- and induces its bioactivity by promoting CXCL13 release by T_{FH}. Our dynamic study and patient longitudinal follow up indicated that T_{FH} TCM pre-existed in blood and tumor beds prior to therapy, while ASC were induced by pembrolizumab and became clinically significant only after injection of therapeutic antibodies. In responding patients, CXCL13-producing CD38^{high}CD2⁺CD4⁺ TILs correlated with B cells that expressed CD86 post-Pembrolizumab. While baseline CD8⁺ TILs were associated with prolonged PFS, TLS required the activity of therapeutic antibodies and coincided with CD38⁺CD28⁺ activated T_{FH} to coordinate the local microenvironment leading to long term PFS.

In breast cancer, Noël et al., highlighted the presence of functional PD1^{high} ICOS⁺ T_{FH} that provided help for the formation of active TLS, characterized by immunoglobulins, proliferating B cells and cytotoxic Th1 CD8⁺ T cell effectors (Noël et al., 2021). Only T_{FH} educated in active TLS had the capacity to provide T-dependent B-cell help and produce IFNγ in ex vivo functional assay (Noël et al., 2021). This is in line with our functional in sitro assay showing that pre-existing T_{FH} correlated with IFNγ producing immune-reactive bladder cancers during exposure to pembrolizumab. The functional significance of T_{FH} to instate tumor immuno-surveillance has been brought up in three elegant mouse models. Hollem et al., dissected the orchestration of immuno-surveillance in triple negative breast cancers harboring a high mutational burden and responding to ICBs (Hollern et al., 2019). They showed that IL-21 producing T_{FH} activated B cells and led to the generation of class-switched plasma cells and tumor-specific IgG, indispensable to control breast tumors. In line with this study, Cui et al., deciphered the interplay between T and B cells using a murine lung adenocarcinoma model in which tumor cells expressed T and B cell-specific neoantigens. They showed that interactions between T_{FH} and GC B cells promoted tumor control by CD8⁺ T cells through an IL-21R-dependent signaling (Cui et al., 2021). In another preclinical model of colorectal cancer devoid of cancer antigens, bacteria-specific T_{FH} were required for the formation of TLS, bacteria-specific cytotoxic T cell responses and control of tumor progression (Overacre-Delgoffe et al., 2021).

In PANDORE study, pre-existing memory CXCL13 producing T_{FH} and B cell cognate immune responses did not appear to be directed against tumor antigens since TMB did not correlate with pathological response nor PFS. Instead, T_{FH} and IgG were directed against commensals common to the digestive and the urinary tracts and/or Gram negative commensals often diagnosed in UTI (such as E. coli) in these patients. Indeed, E. coli IgG titers predicted long term clinical benefit (prolonged PFS) to pembrolizumab in two independent cohorts of patients. The immuno-stimulatory capacity of ICBs depends on the taxonomic composition of the intestinal microbiota (Gopalakrishnan et al., 2018; Matson et al., 2018; Routy et al., 2018; Sivan et al., 2015; Vétizou et al., 2015). UTI are the most common bacterial infections, afflicting women, elderly persons as well as cancer patients (Flores-Mireles et al., 2015). UTI are typically initiated when certain gut-derived bacteria, such as uropathogenic E. coli reach the bladder and invade urothelial cells. Several reports identified uropathogenic E. coli antigens evoking robust humoral responses that significantly reduced bladder and kidney infections upon bacterial challenge (Abraham et al., 1988; Asadi Karam et al., 2013). Cellular Th1 immune responses elicited through mucosal immunization with E. coli antigens were shown to be protective against bacterial rechallenge (Wu et al., 2021, p. 1). Moreover, urine lymphocytes may accurately map the tumor immune landscape in locally advanced UC patients treated with ICBs, and could predict recurrence-free survival (Wong et al., 2018). While the human tumor microbiome is being deconvoluted (Nejman et al., 2020; Sepich-Poore et al., 2021), new prospects for TIL generation and transfer appear to rely on exploiting MHC-restricted T cell responses specifically directed toward intra-tumoral bacteria (Kalaora et al., 2021). Bacteria inhabiting the mucus layer close to the intestinal epithelium preferentially induce T_{FH} (Ansaldo et al., 2019; Roberti et al., 2020; Xu et al., 2018). Our study showed that MIBC can be invaded by a distinct commensalism comprising bacteria capable of eliciting specific T_{FH} and B cell responses. After pembrolizumab therapy, E. coli-specific CXCL13-producing T_{FH} tended to decrease, presumably to home to tumor lesions where they are expected to orchestrate the immune landscape of the TME for the long-term clinical protection against relapse. We failed to isolate UTI-related bacteria such as E. coli from our fresh bladder tissues culturomics and mass spectrometric analyses while succeeding in isolating Gram positive bacteria (such as S. capitis already described in melanoma (Kalaora et al., 2021)). Work is in progress to unveil how intra-urothelial bacteria can be “cross-presented” into tumor MHC molecules to become elective targets of T_{FH} or CD8⁺ TILs. Pioneering work from Conejo-Garcia's group unraveled the biological significance of transcytosis of tumor antigen specific- and non-specific-IgA in the control of ovarian cancer (Biswas et al., 2021).

These results emphasized that uropathogenic commensals, notably uropathogenic E. coli (UPEC) endowed with intrinsic immunogenicity and with tumor invasion capacities may be harnessed for a better efficacy of ICBs and should be viewed as potential biomarkers of response to ICBs. According to this assumption, the development of UPEC-based vaccines to elicit a specific T-B crosstalk prior to ICBs administration deserves some attention and preclinical assessment. Along the same lines, extending BCG instillations beyond their current indications (for instance including MIBC) may be instrumental prior to ICBs to boost BCG vaccine-induced immunity and offer MHC-binding targets for CD8⁺ effectors.

Regardless of these considerations, monitoring circulating T_{FH} immune responses directed against tumor or commensal antigens should be integrated in the armamentarium of the promising predictors of clinical benefit to ICBs-based therapies in tumors located in skin or mucosae in the future.

### Example 7: Bacteria-Specific IgG Detection, Isotyping and Sequencing of Immunoglobulins from MIBT

MIBC tumor tissues or non-tumor tissues are split in two parts, one part will be stored frozen at −80° C. while the second part is technically processed freshly as follows.

MIBC tumor tissues or non-tumor tissues are dissociated into single-cell suspensions by combining mechanical dissociation with enzymatic degradation of the extracellular matrix. The tumor(s)/non-tumor tissue(s) are enzymatically digested using the human kit components without the “R” kit enzyme (Miltenyi CAT #130-095-929) and the gentleMACS™ Dissociators (Miltenyi) are used for the mechanical dissociation steps. After dissociation, the sample(s) is (are) applied to a filter to remove any remaining larger particles from the single-cell suspension. The number of CD45⁺ cells by sample is determined from un aliquot using precision count beads (Biolegend Cat #424902) combined with CD45-FITC (BD, CAT #555482) and 7ADD (live/dead Biolegend, CAT #420404).

Single-cell suspension(s) is (are) resuspended in complete culture media, LONZA X-VIVO 15 (CAT #BEBP02-054Q) supplemented with Peni-Streptomycine (Gibco, CAT #15140-122) at the final concentration of 0.56×10⁶ CD45⁺ cells/ml. X-VIVO™ 15 Medium provides a serum-free environment optimized to support the growth of human monocytes, macrophage cells and cell lines, PBL, granulocytes and natural killer (NK) cells.

Single-cell suspension(s) is (are) plated dispensing 180 μl/well in 96-wells flat bottom plate, i.e., 10⁵ CD45⁺ cells/well and stimulated under different conditions: (i) mixture of [1 μg/ml]R848 (Invivogen, CAT #tlr-R848) and [10³ U/ml] IL-2; (ii) Hu-B-Poly-SE (ImmunoSpot, CAT #CTL-HBPOLYSE-35) final dilution 1:333 according to the manufacturer recommendations; (iii) heat-inactivated bacteria 1.5×10⁵ bacteria/ml or complete culture media alone, used as negative control. Then, plates are incubated at 37° C., 5% CO₂. After 70-72 Hrs of incubation, plates are centrifuged 5 min at 450 g, and supernatants are collected and frozen at −20° C.

Isotyping of immunoglobulins from culture supernatant is performed using Procartaplex Hu-Ab Isotyping, panel 7plex kit (Thermofisher, CAT #EPX070-10818-901) according to the manufacturer's recommendations. According to the ability of different stimulation conditions to induce immunoglobulin secretion from tumor infiltrating B cells, the antigen specificity against bacteria is determined using bacterial flow cytometry assay (see protocol in Materials and Methods).

Before in vitro stimulation, immunophenotyping is performed from single-cell suspension to determine the proportion of T_{FH}, TREGs, TRM-CD8 T cells and B cells (Memory; GC; and Plasmocytes) in order to establish correlations with secreted antibody profile following different stimulation conditions and the immune contexture. A culture is considered “positive” when B cells get activated (CD69, CD40, Ig secretion).

| | Target | Fluorochrome | Clone | Supplier | Ref# |
|---|---|---|---|---|---|
| 1 | CXCR5 (CD185) | bb700 | RF8B2 | BD | 566469 |
| 2 | CD3 | BUV395 | UCHT1 | BD | 563546 |
| 3 | CD4 | BUV496 | SK3 | BD | 612936 |
| 4 | CD127 | BV421 | HIL-7R- | BD | 562436 |
| | | | M21 | | |
| 5 | CD25 | BV650 | BC96 | BIOLEGEND | 3026334 |
| 6 | CD183 (CXCR3) | PE | G025H7 | BIOLEGEND | 353706 |
| 7 | CD278 (ICOS) | BUV737 | DX29 | BD | 749665 |
| 8 | CD8 | APC-H7 | SK1 | BD | 560179 |
| 9 | CD38 | BV711 | HB7 | BD | 740830 |
| 10 | CD69 | PeCy7 | FN50 | BIOLEGEND | 310912 |
| 11 | CD19 | PE/Dazzle 594 | HIB19 | BD | 562294 |
| 12 | CD103 | BV605 | BerACT8 | BIOLEGEND | 350218 |
| 13 | CD27 | APC-R700 | M-T271 | BD | 565116 |
| 14 | CD45 | BUV805 | HI30 | BD | 612891 |
| 15 | PD1 | BV786 | EH12.1 | BD | 563789 |
| 16 | IgD | FITC | IA6-2 | BD | 562023 |
| 17 | CD40 | APC | 77220 | BIOLEGEND | 334310 |
| 18 | Zombie Acqua | BUV510 | | BIOLEGEND | 4231101 |

Additionally, to complete the correlation matrices between immune contexture and secreted antibody profile from culture supernatant, a panel of 15 immune soluble factors released post in-vitro stimulation is quantified using custom Procartaplex kit (Thermofisher, CAT #FYDZ-PPX15MXM) encompassing: IFNg; IL-10; IL-13; IL-17; IL-21; IL-22; IL-4; IL-5; IL-6; IL-8; TNFa; CXCL-13; CXCL-10; CCL-20 and CCL-22. A culture is considered “positive” when IL-21 or CXCL13 is produced, hallmarks of T_{FH}.

When a culture is considered positive, the other half of the tumor tissue or non tumor tissue that has been stored at −80° C. is thawed, restimulated in the same condition (when considered positive in the first phase), and after 24 hrs, all B cells are cell-sorted with Facs cell sorter, and each single cell transcript is sequenced; whole exon sequencing is also performed of each activated B cell.

The Ig sequence is then genetically inserted into a vector to transduce CHO producing cells, as known by the skilled person for upscaled protein production.

### Example 8: Automated Test for Direct and Indirect Assessment of the Presence of Bacteria-Specific T_{FH}

Using a simple 22 hr-whole blood stimulation assay (see Materials and Methods, 5.4) allowing the quantitative measurement of IFN-γ or CXCL9 using the Enzyme Linked Fluorescent Assay technique in an automated platform (VIDAS® IFN-γ RUO, (Mouton et al., 2021), we analyze E. coli or S. capitis-specific T-cell reactivities.

We use non overlapping 15-mer peptides covering distinct E. coli nucleic acid sequences. The concentration of IFN-γ or CXCL9 in the supernatant is measured using the VIDAS automated platform (VIDAS® IFN-γ RUO, bioMérieux). The positivity range is 0.08-8 IU/mL and IFA positivity (positive control) thresholds are defined at 0.08 IU/mL and 8 IU, respectively. The IFN-γ response is defined as positive when the IFNγ concentration of the test is above threshold and the negative control is below threshold or when the IFNγ concentration of the test minus IFN-γ concentration of the negative control is above threshold. All positive controls are ≥8 IU/mL.

### Example 9: E. coli Reactivates Antitumor T Lymphocytes

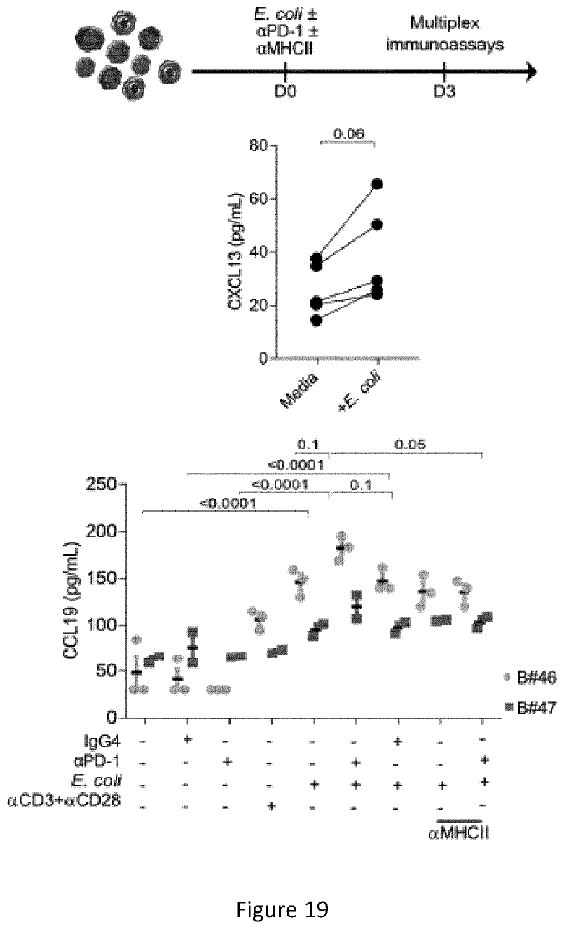

In this example, in sitro assays were performed on fresh muscle invasive bladder cancers (MIBC) in the presence of bacteria, to analyze the in situ reactivity to E. coli with or without pembrolizumab (FIG. 19, upper panel), and thereby demonstrate that the local microbiotherapy described herein could be beneficial and efficient to reinstate cancer immunosurveillance.

### Material and Methods for in Sitro Tumor Assays

In sitro assays were performed as described in § 4 of the Materials and Methods described above, with the following complements.

For in sitro assays using bacteria, cells were resuspended in an appropriate volume of TheraPEAK™ X-VIVO™-15 Serum-free Hematopoietic Cell Medium (TheraPEAK™ X-VIVO™-15, LONZA, Cat #BEBP02-061Q) supplemented with Penicillin/Streptomycin (GIBCO Invitrogen, Cat #15140-122) extemporaneously and plated at 1×10⁵ CD45⁺ cells/well in 96-well U-bottom plate. Stimulation agents (heat-killed Escherichia coli Q1696 (3×10⁶/well), heat-killed Staphylococcus capitis (3×10⁶/well), R848 (Invivogen, Cat #tlrl-r848) (final concentration: 1 μg/mL)+IL-2 (PeproTech, Cat #200-02-11) (final concentration: 1×10³ U/mL), IL-21+IL-4+anti-CD40 (Human B-Poly-SE™, Immunospot, Cat ##CTL-hBPOLYSE-35) were prepared extemporaneously and added to cell culture before incubation at 37° C. in a humidified atmosphere of 5% CO₂ during 3 days. CXCL13 and IgG/A were monitored suing commercial ELISA.

### Bacterial Strains and Culture Conditions

Cultivable bacteria, relevant in bladder cancer patients, Escherichia coli (Q1696, pks positive, IHU Marseille, urine from non-cancer patient) or Staphylococcus capitis (feces from kidney cancer patient non-responder to ICBs, EverImmune) were plated onto sheep's blood agar plates (COS, BioMérieux) at 37° C. with 5% CO₂ in anaerobic or aerobic conditions for 48 h and identified by Matrix Assisted Laser Desorption Ionization-Time of Flight (MALDI-TOF) mass spectrometry (Andromas; Beckman Coulter, Brea, California USA).

### Results

The ultimate goal of the therapeutic use of E. coli disclosed herein is to reactivate MHC class II-restricted, E. coli (UPEC)-specific memory follicular T helper cells in situ in the bladder tumor microenvironment during PD-1 blockade. To do so, we aim at a local in situ delivery of inactivated E. coli or UPEC in individual patients prior to starting anti-PD-1 Ab.

To show that this local microbiotherapy could be beneficial and efficient to reinstate cancer immunosurveillance, we analyzed the in situ reactivity to E. coli with or without pembrolizumab in in sitro assays on fresh muscle invasive bladder cancers (MIBC) (FIG. 19, upper panel). After 72 hours of exposure to heat inactivated E. coli, we observed a release of CXCL13 (FIG. 19, middle panel) and an MHC class II-restricted release of tertiary lymphoid structure (TLS)-associated CCL19 chemokine (FIG. 19, bottom panel). These two reactivities (CXCL13 and CCL19) upon exposure to E. coli, shown in independent patients' tumors, clearly bring the proof-of-concept that local delivery of heat killed or pasteurized UPEC (strain Q1696 expressing pks, the colibactin) can reactivate TFH expressing PD-1, paving the way to exacerbated anticancer responses to pembrolizumab (anti-PD1 Abs).

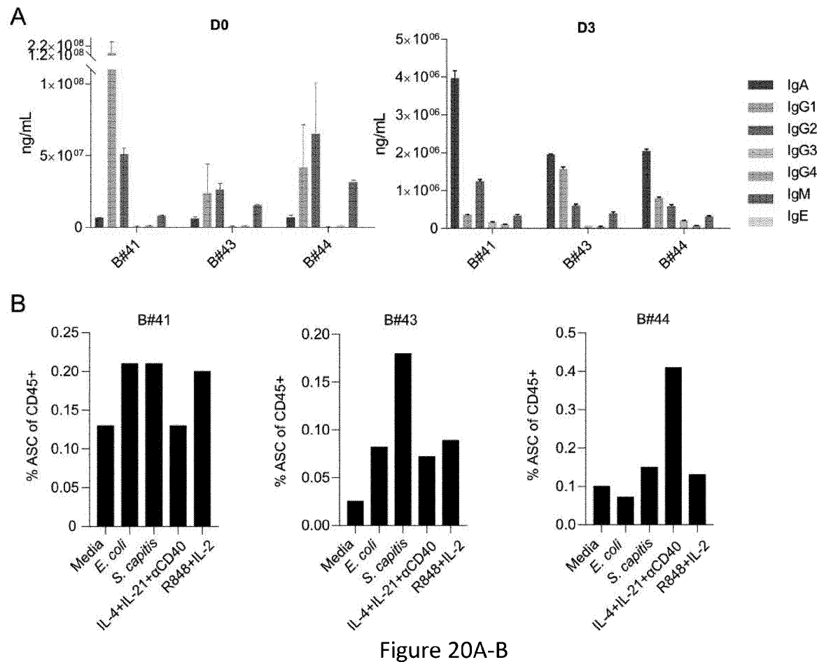

Release of of CXCL13 and CCL19 from the fresh MIBC triggered by E. coli also brings the proof-of-concept that local delivery of heat killed or pasteurized UPEC (strain Q1696 expressing pks, the colibactin) can reactivate the cross talk between TFH and B cells, culminating in the activation and proliferation of antibody (IgG1, IgG2, IgA) secreting cells (plasmocytes). Of note, this reactivity could be obtained against E. coli that were not colibactin positive (pks negative, not shown) but also, in rare cases, against Staphylococcus capitis. In these fresh in situ cocultures, antibody secreting cell numbers increased in the cocultures (FIG. 20). Moreover, this release was amplified by addition of pembrolizumab.

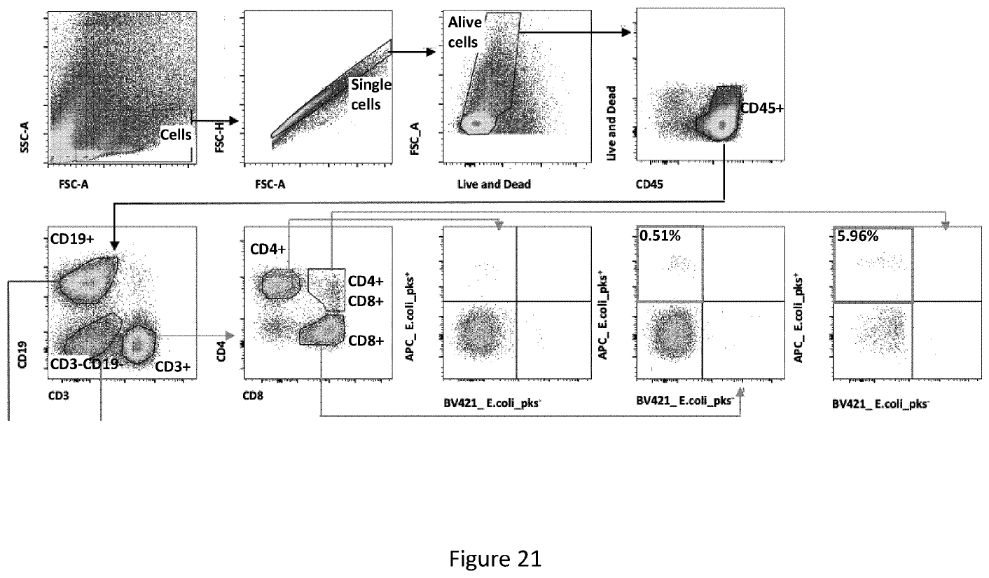

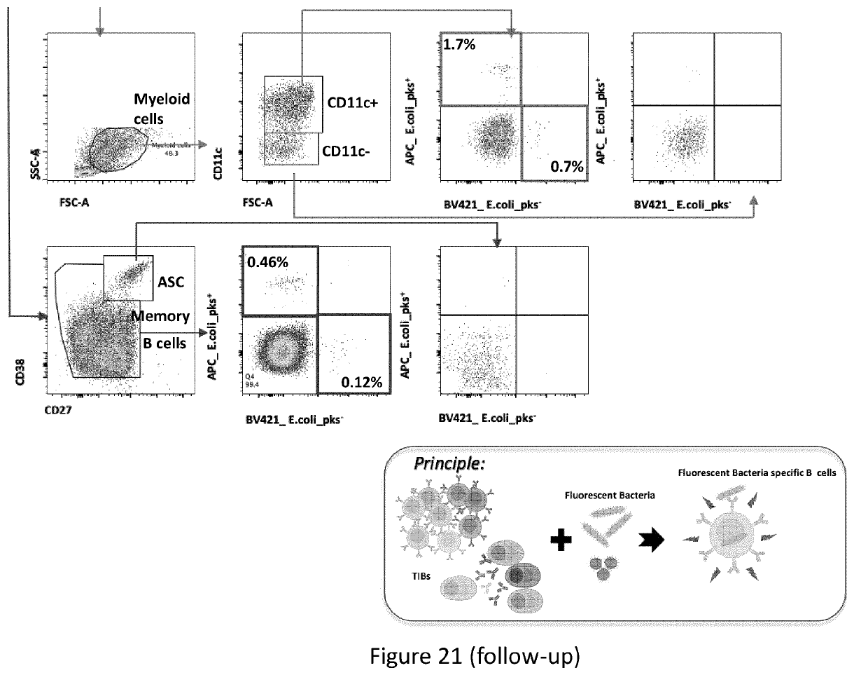

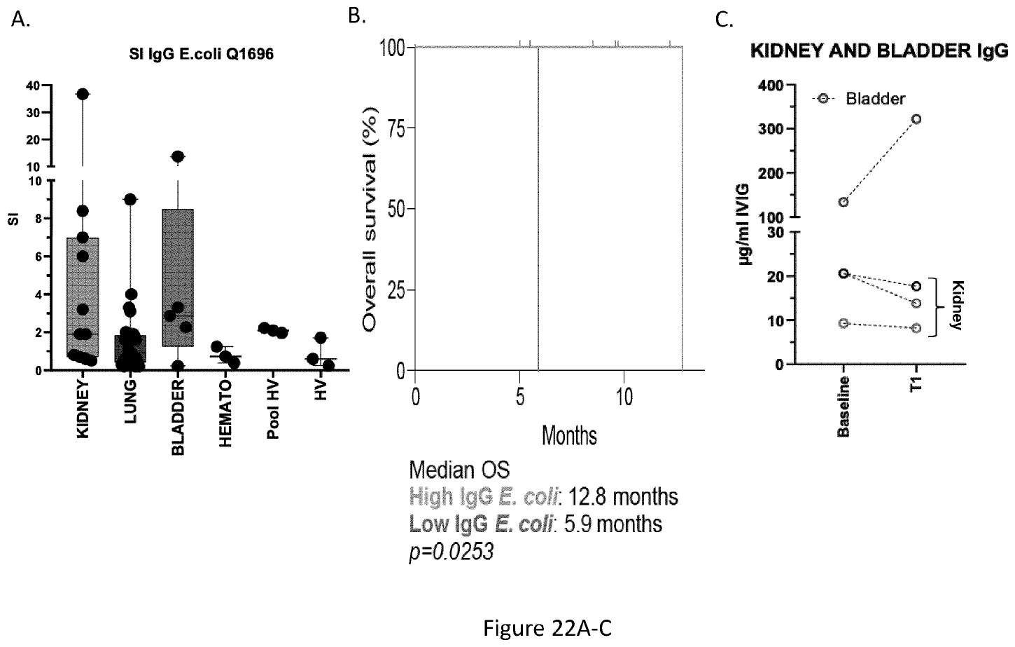

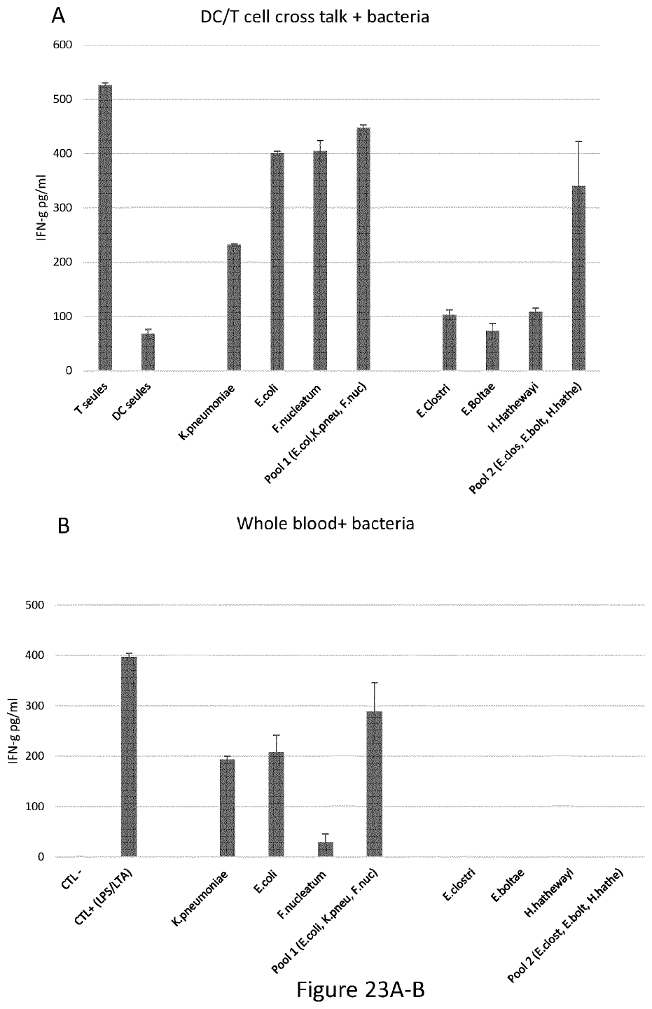

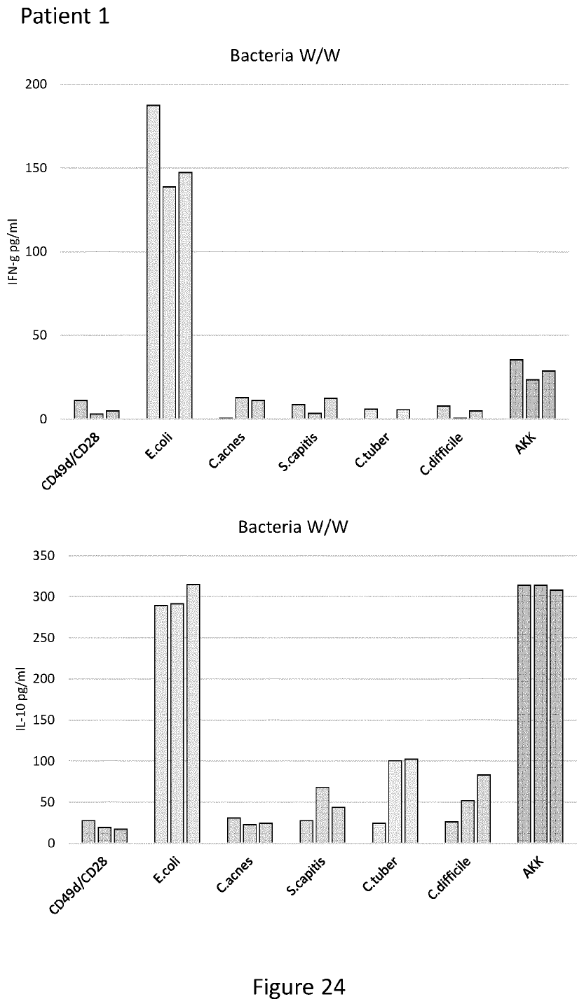

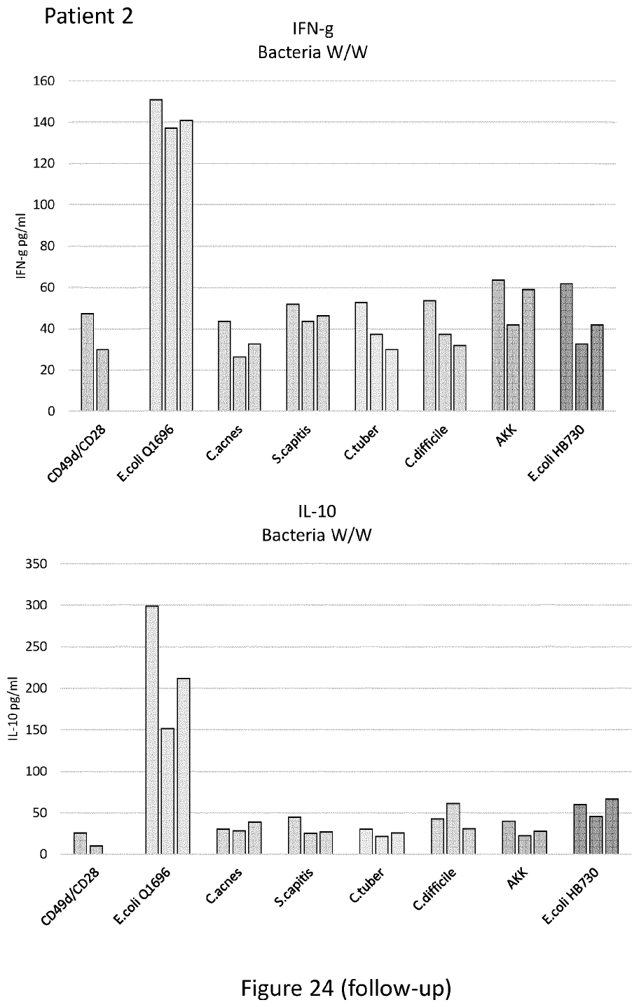

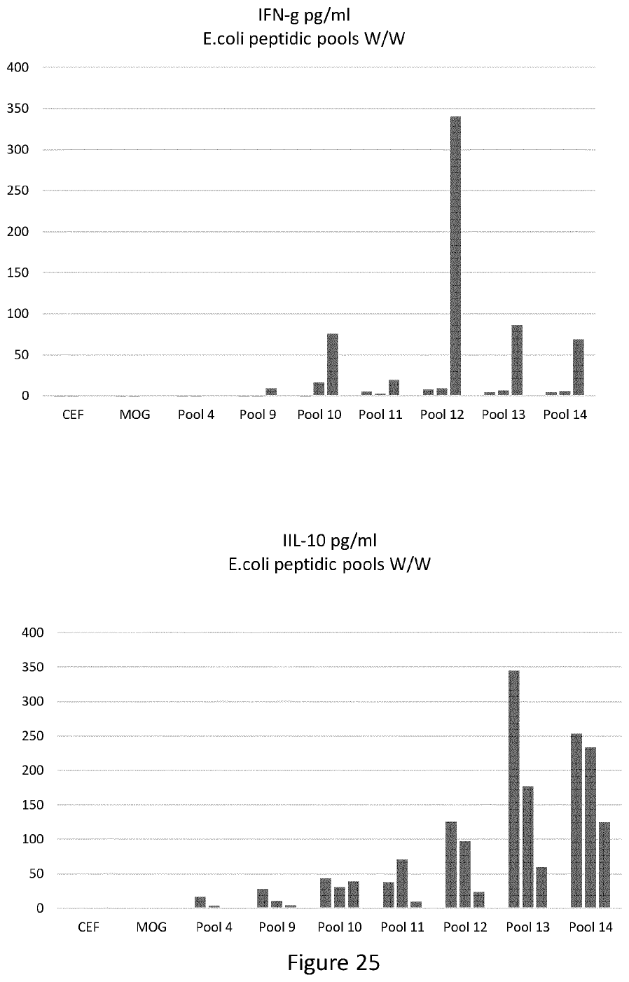

Finally, a fresh tumor sample has been incubated with fluorescent E coli (commercially available) that could bind to E. coli-specific memory B cells. Hence, we could quantify by flow cytometry analysis gating on memory or early activated memory B cells, those that were infiltrating the tumor (FIG. 21).

### Example 10: E. coli-Specific IgG and T Lymphocytes are Biomarkers for Predicting the Response of an Anti-PD-1 Treatment Both in Bladder and Kidney Cancers

This example illustrates that the predictive value (for the response to PD-1 blockade) of the E. coli-specific IgG titers can be extended to kidney cancers. The results were obtained on patients from the NIVOREN protocol. We monitored these seroreactivities in the serum of 15 metastatic renal cell cancer patients at baseline prior to 2 L anti-PD-1 Ab therapy by flow cytometric analysis according to the method described above and by Goubet et al. (Cancer Discovery 2022). As shown in FIG. 22, the staining indices were higher in bladder and renal cell cancer patients compared with lung cancers or hematological malignancies or healthy volunteers (FIG. 22, left panel). Here again, as observed in MIBC, the E. coli-specific IgG staining indices predicted overall survival in second line pembrolizumab (middle panel), high levels above the median of the cohort being of favorable prognosis. Interestingly, these serum titers are pretty stable over time, meaning that baseline titers or seroreactivities measured during therapy are not significantly different (right panel).

In addition, we have transformed the tedious and long (requiring 9 days) DC/T cell assay described in Example 6 above and in Goubet et al. (Cancer Discovery, 2022) into a practical and rapid assay (less than 24 hrs) using fresh blood incubated with inactivated or pasteurized bacteria (one or a pool of several bacteria), including E. coli. We show that the results are comparable in several donors (FIG. 23).

We show in two cancer patients (MIBC) that the T cell reactivity (monitored in IFNγ or IL-10) is sky high against E. coli compared with all the other bacteria (urinary commensals or not like Clostridium difficile) (FIG. 24).

Similar Method but Pulsing Long Peptides Rather than Pasteurized Bacteria

Finally, we could eventually replace whole pasteurized E. coli by peptide pools (9-15-mer) covering antigenic sequences already described for E. coli in the literature such FimH or lutA (FIG. 25 and Table 8). This test is a friendly user assay that could be implemented into clinical practice using, for instance the VIDAS Biomerieux technology.

TABLE 8
Detailed composition of the
pools used in the experiments
illustrated in FIG. 25

| | | | SEQ |
|---|---|---|---|
| | | | ID |
| | | sequence | No |
| Pool 4 | E.coli | LLGGLLTMV | 1 |
| Pool 9 | E.coli | PRVVYNSRTDKPWPV | 2 |
| (FimH 20-24) | | YNSRTDKPWPVALYL | 3 |
| | | TDKPWPVALYLTPVS | 4 |
| | | PWPVALYLTPVSSAG | 5 |
| | | ALYLTPVSSAGGVAI | 6 |
| Pool 10 | E.coli | AGSLIAVLILRQTNN | 7 |
| (FimH 28-30) | | IAVLILRQTNNYNSD | 8 |
| | | ILRQTNNYNSDDFQF | 9 |
| Pool 9 | E.coli | PRVVYNSRTDKPWPV | 10 |
| (FimH 20-24) | | YNSRTDKPWPVALYL | 11 |
| | | TDKPWPVALYLTPVS | 12 |
| | | PWPVALYLTPVSSAG | 13 |
| | | ALYLTPVSSAGGVAI | 14 |
| Pool 10 | E.coli | AGSLIAVLILRQTNN | 15 |
| (FimH 28-30) | | IAVLILRQTNNYNSD | 16 |
| | | ILRQTNNYNSDDFQF | 17 |
| Pool 11 | E.coli | LVGQVYYRDESLRFY | 18 |
| (lutA 1.1) | | | |
| Pool 12 | E.coli | FPTVNANKQATAFSS | 19 |
| (lutA 1.5-1.6) | | NANKQATAFSSSQQ | 20 |
| Pool 13 | E.coli | SRLRHFLFNAGLLMH | 21 |
| (lutA 2.1) | | | |
| Pool 14 | E.coli | GWRFTGDNLRTQIAA | 22 |
| (lutA 2.22-2.27) | | TGDNLRTQIAAYYSL | 23 |
| | | LRTQIAAYYSLSNKS | 24 |
| | | IAAYYSLSNKSVERN | 25 |
| | | YSLSNKSVERNKDLT | 26 |
| | | NKSVERNKDLTISVK | 27 |
| | | ERNKDLTISVKDDRR | 28 |

Conclusion: Altogether, we now show that E. coli-specific IgG titers or seroreactivities can be monitored by flow cytometric analyses at baseline (or during therapy) and/or E. coli or E. coli peptide-specific recall T cell responses can be monitored by a rapid 24 hrs whole blood assay to identify patients (bladder or kidney cancer patients) proned to benefit from pembrolizumab.

Abbreviations Used in this Text:

ASC: Antibody-secreting cell, BCG: Bacillus Calmette-Guérin, CI: confidence interval, CPS: Combined Positive Score, CyTOF: cytometry by time-of-flight, E. coli: Escherichia coli, FISH: fluorescent in situ hybridization, HES: hematoxylin-eosin-safran, ICBs: Immune Checkpoint Blockers, IQR: interquartile range, MFI: median fluorescence intensity, MIBC: muscle invasive bladder cancer, MPR: major pathological response, OS: Overall survival, mAbs: monoclonal antibodies, pCR: pathological complete response, PFS: Progression-free survival, PD-1: Programmed cell death protein 1, PD-L1: Programmed cell death ligand 1, TCM: Central Memory T cells, TE: Effector T cells, TEM: Effector Memory T cells, T_{FH}: Follicular helper CD4⁺ T cells, TILs: tumor-infiltrating lymphocytes, TLS: tertiary lymphoid structure, TMB: Tumor Mutational Burden, TME: Tumor microenvironment, TRM: tissue resident memory, TURBT: transurethral resection for bladder tumor, UC: Urothelial carcinoma, UPEC: Uropathogenic Escherichia coli, UTI: urinary tract infection.

### REFERENCES

Abraham, S. N., D. Sun, J. B. Dale, et E. H. Beachey. 1988. «Conservation of the D-Mannose-Adhesion Protein among Type 1 Fimbriated Members of the Family Enterobacteriaceae». Nature 336 (6200): 682-84.
Alfred Witjes, J., Thierry Lebret, Eva M. Compérat, Nigel C. Cowan, Maria De Santis, Harman Maxim Bruins, Virginia Hernández, et al. 2017. «Updated 2016 EAU Guidelines on Muscle-Invasive and Metastatic Bladder Cancer». European Urology 71 (3): 462-75.
Ansaldo, Eduard, Leianna C. Slayden, Krystal L. Ching, Meghan A. Koch, Natalie K. Wolf, Damian R. Plichta, Eric M. Brown, et al. 2019. «Akkermansia Muciniphila Induces Intestinal Adaptive Immune Responses during Homeostasis». Science (New York, N.Y.) 364 (6446): 1179-84.
Asadi Karam, Mohammad Reza, Mana Oloomi, Mehdi Mahdavi, Mehri Habibi, et Saeid Bouzari. 2013. «Vaccination with Recombinant FimH Fused with Flagellin Enhances Cellular and Humoral Immunity against Urinary Tract Infection in Mice». Vaccine 31 (8): 1210-16.
Bajorin, Dean F., J. Alfred Witjes, Jürgen E. Gschwend, Michael Schenker, Begoña P. Valderrama, Yoshihiko Tomita, Aristotelis Bamias, et al. 2021. «Adjuvant Nivolumab versus Placebo in Muscle-Invasive Urothelial Carcinoma». The New England Journal of Medicine 384 (22): 2102-14.
Balar, Arjun V., Daniel Castellano, Peter H. O'Donnell, Petros Grivas, Jacqueline Vuky, Thomas Powles, Elizabeth R. Plimack, et al. 2017. «First-Line Pembrolizumab in Cisplatin-Ineligible Patients with Locally Advanced and Unresectable or Metastatic Urothelial Cancer (KEYNOTE-052): A Multicentre, Single-Arm, Phase 2 Study». The Lancet. Oncology 18 (11): 1483-92.
Balar, Arjun V., Matthew D. Galsky, Jonathan E. Rosenberg, Thomas Powles, Daniel P. Petrylak, Joaquim Bellmunt, Yohann Loriot, et al. 2017. «Atezolizumab as First-Line Treatment in Cisplatin-Ineligible Patients with Locally Advanced and Metastatic Urothelial Carcinoma: A Single-Arm, Multicentre, Phase 2 Trial». Lancet (London, England) 389 (10064): 67-76.
Balar, Arjun V., Ashish M. Kamat, Girish S. Kulkarni, Edward M. Uchio, Joost L. Boormans, Mathieu Roumiguié, Laurence E. M. Krieger, et al. 2021. «Pembrolizumab Monotherapy for the Treatment of High-Risk Non-Muscle-Invasive Bladder Cancer Unresponsive to BCG (KEYNOTE-057): An Open-Label, Single-Arm, Multicentre, Phase 2 Study». The Lancet. Oncology 22 (7): 919-30.
Bankhead P, Loughrey M B, Fernández J A, Dombrowski Y, McArt D G, Dunne P D, et al. QuPath: Open source software for digital pathology image analysis. Sci Rep. 2017; 7:16878.
Bellmunt, Joaquim, Ronald de Wit, David J. Vaughn, Yves Fradet, Jae-Lyun Lee, Lawrence Fong, Nicholas J. Vogelzang, et al. 2017. «Pembrolizumab as Second-Line Therapy for Advanced Urothelial Carcinoma». New England Journal of Medicine 376 (11): 1015-26.
Biswas, Subir, Gunjan Mandal, Kyle K. Payne, Carmen M. Anadon, Chandler D. Gatenbee, Ricardo A. Chaurio, Tara Lee Costich, et al. 2021. «IgA Transcytosis and Antigen Recognition Govern Ovarian Cancer Immunity». Nature 591 (7850): 464-70.
Briganti, Alberto, Giorgio Gandaglia, Simone Scuderi, Andrea Gallina, Renzo Colombo, Nicola Fossati, Francesco Barletta, et al. 2020. «Surgical Safety of Radical Cystectomy and Pelvic Lymph Node Dissection Following Neoadjuvant Pembrolizumab in Patients with Bladder Cancer: Prospective Assessment of Perioperative Outcomes from the PURE-01 Trial». European Urology 77 (5): 576-80.
Brons, Jolanda K., Stefanie N. Vink, Marjon G. J. de Vos, Stefan Reuter, Ulrich Dobrindt, et Jan Dirk van Elsas. 2020. «Fast Identification of Escherichia Coli in Urinary Tract Infections Using a Virulence Gene Based PCR Approach in a Novel Thermal Cycler». Journal of Microbiological Methods 169 (février): 105799.
Brubaker, Linda, et Alan Wolfe. 2016. «The Urinary Microbiota: A Paradigm Shift for Bladder Disorders?«Current opinion in obstetrics & gynecology 28 (5): 407-12.
Cabrita, Rita, Martin Lauss, Adriana Sanna, Marco Donia, Mathilde Skaarup Larsen, Shamik Mitra, Iva Johansson, et al. 2020. «Tertiary Lymphoid Structures Improve Immunotherapy and Survival in Melanoma». Nature 577 (7791): 561-65.
Cui, Can, Jiawei Wang, Eric Fagerberg, Ping-Min Chen, Kelli A. Connolly, Martina Damo, Julie F. Cheung, et al. 2021. «Neoantigen-Driven B Cell and CD4 T Follicular Helper Cell Collaboration Promotes Anti-Tumor CD8 T Cell Responses». Cell 184 (25): 6101-6118.e13.
Daillère, Romain, Marie Vétizou, Nadine Waldschmitt, Takahiro Yamazaki, Christophe Isnard, Vichnou Poirier-Colame, Connie P. M. Duong, et al. 2016. «Enterococcus Hirae and Barnesiella Intestinihominis Facilitate Cyclophosphamide-Induced Therapeutic Immunomodulatory Effects». Immunity 45 (4): 931-43.
Dijk, Nick van, Alberto Gil-Jimenez, Karina Silina, Kees Hendricksen, Laura A. Smit, Jeantine M. de Feijter, Maurits L. van Montfoort, et al. 2020. «Preoperative Ipilimumab plus Nivolumab in Locoregionally Advanced Urothelial Cancer: The NABUCCO Trial». Nature Medicine 26 (12): 1839-44.
Dubourg, Grégory, Aurélie Morand, Fatima Mekhalif, Raphael Godefroy, Alice Corthier, Abdourahamane Yacouba, Ami Diakite, et al. 2020. «Deciphering the Urinary Microbiota Repertoire by Culturomics Reveals Mostly Anaerobic Bacteria From the Gut». Frontiers in Microbiology 11: 513305.
Dubuisson, Agathe, Jean-Eudes Fahrner, Anne-Gaëlle Goubet, Safae Terrisse, Nicolas Voisin, Charles Bayard, Sebastien Lofek, et al. 2021. «Immunodynamics of Explanted Human Tumors for Immuno-Oncology». EMBO Molecular Medicine 13 (1): e12850.
Eggermont, Alexander M. M., Giovanni Apolone, Michael Baumann, Carlos Caldas, Julio E. Celis, Francesco de Lorenzo, Ingemar Ernberg, et al. 2019. «Cancer Core Europe: A Translational Research Infrastructure for a European Mission on Cancer». Molecular Oncology 13 (3): 521-27.
Flores-Mireles, Ana L., Jennifer N. Walker, Michael Caparon, et Scott J. Hultgren. 2015. «Urinary tract infections: epidemiology, mechanisms of infection and treatment options». Nature reviews. Microbiology 13 (5): 269-84.
Gao, Jianjun, Neema Navai, Omar Alhalabi, Arlene Siefker-Radtke, Matthew T. Campbell, Rebecca Slack Tidwell, Charles C. Guo, et al. 2020. «Neoadjuvant PD-L1 plus CTLA-4 Blockade in Patients with Cisplatin-Ineligible Operable High-Risk Urothelial Carcinoma». Nature Medicine 26 (12): 1845-51.
Gopalakrishnan, V., C. N. Spencer, L. Nezi, A. Reuben, M. C. Andrews, T. V. Karpinets, P. A. Prieto, et al. 2018. «Gut Microbiome Modulates Response to Anti-PD-1 Immunotherapy in Melanoma Patients». Science (New York, N.Y.) 359 (6371): 97-103.
Goswami, Sangeeta, Yulong Chen, Swetha Anandhan, Peter M. Szabo, Sreyashi Basu, Jorge M. Blando, Wenbin Liu, et al. 2020. «ARID1A Mutation plus CXCL13 Expression Act as Combinatorial Biomarkers to Predict Responses to Immune Checkpoint Therapy in MUCC». Science Translational Medicine 12 (548): eabc4220.
Goubet A G, et al. 2022. Escherichia coli-Specific CXCL13-Producing TFH Are Associated with Clinical Efficacy of Neoadjuvant PD-1 Blockade against Muscle-Invasive Bladder Cancer. Cancer Discov. October 5; 12(10):2280-2307. doi: 10.1158/2159-8290.CD-22-0201. PMID: 35929803.
Graham L, Orenstein J M. Processing tissue and cells for transmission electron microscopy in diagnostic pathology and research. Nat Protoc. 2007; 2:2439-50.
Grine G, Terrer E, Boualam M A, Aboudharam G, Chaudet H, Ruimy R, et al. Tobacco-smoking-related prevalence of methanogens in the oral fluid microbiota. Sci Rep. 2018; 8:9197.
Gu-Trantien, Chunyan, Edoardo Migliori, Laurence Buisseret, Alexandre de Wind, Sylvain Brohée, Soizic Garaud, Grégory Noël, et al. 2017. «CXCL13-Producing TFH Cells Link Immune Suppression and Adaptive Memory in Human Breast Cancer». JCI Insight 2 (11): 91487.
Hamid, Omid, Caroline Robert, Adil Daud, F. Stephen Hodi, Wen-Jen Hwu, Richard Kefford, Jedd D. Wolchok, et al. 2013. «Safety and Tumor Responses with Lambrolizumab (Anti-PD-1) in Melanoma». The New England Journal of Medicine 369 (2): 134-44.
Hasanzadeh, S., Habibi, M., Shokrgozar, M. A. et al. In silico analysis and in vivo assessment of a novel epitope-based vaccine candidate against uropathogenic Escherichia coli. Sci Rep 10, 16258 (2020).
Helmink, Beth A., Sangeetha M. Reddy, Jianjun Gao, Shaojun Zhang, Rafet Basar, Rohit Thakur, Keren Yizhak, et al. 2020. «B Cells and Tertiary Lymphoid Structures Promote Immunotherapy Response». Nature 577 (7791): 549-55.
Herbst, Roy S., Jean-Charles Soria, Marcin Kowanetz, Gregg D. Fine, Omid Hamid, Michael S. Gordon, Jeffery A. Sosman, et al. 2014. «Predictive Correlates of Response to the Anti-PD-L1 Antibody MPDL3280A in Cancer Patients». Nature 515 (7528): 563-67.
Hollern, Daniel P., Nuo Xu, Aatish Thennavan, Cherise Glodowski, Susana Garcia-Recio, Kevin R. Mott, Xiaping He, et al. 2019. «B Cells and T Follicular Helper Cells Mediate Response to Checkpoint Inhibitors in High Mutation Burden Mouse Models of Breast Cancer». Cell 179 (5): 1191-1206.e21.
Huang, Alexander C., Michael A. Postow, Robert J. Orlowski, Rosemarie Mick, Bertram Bengsch, Sasikanth Manne, Wei Xu, et al. 2017. «T-cell invigoration to tumour burden ratio associated with anti-PD-1 response». Nature 545 (7652): 60-65.
Ishida, Y., Y. Agata, K. Shibahara, et T. Honjo. 1992. «Induced Expression of PD-1, a Novel Member of the Immunoglobulin Gene Superfamily, upon Programmed Cell Death». The EMBO Journal 11 (11): 3887-95.
Jacquelot, N., M. P. Roberti, D. P. Enot, S. Rusakiewicz, N. Ternès, S. Jegou, D. M. Woods, et al. 2017. «Predictors of Responses to Immune Checkpoint Blockade in Advanced Melanoma». Nature Communications 8 (1): 592.
Jiang, X., J. E. Castelao, S. Groshen, V. K. Cortessis, D. Shibata, D. V. Conti, J.-M. Yuan, M. C. Pike, et M. Gago-Dominguez. 2009. «Urinary Tract Infections and Reduced Risk of Bladder Cancer in Los Angeles». British Journal of Cancer 100 (5): 834-39.
Jones, Gareth W., David G. Hill, et Simon A. Jones. 2016. «Understanding Immune Cells in Tertiary Lymphoid Organ Development: It Is All Starting to Come Together». Frontiers in Immunology 7: 401.
Jordan, Brian, et Joshua J. Meeks. 2019. «T1 Bladder Cancer: Current Considerations for Diagnosis and Management». Nature Reviews. Urology 16 (1): 23-34.
Kalaora, Shelly, Adi Nagler, Deborah Nejman, Michal Alon, Chaya Barbolin, Eilon Barnea, Steven L. C. Ketelaars, et al. 2021. «Identification of Bacteria-Derived HLA-Bound Peptides in Melanoma». Nature 592 (7852): 138-43.
Lawrence M S, Stojanov P, Polak P, Kryukov G V, Cibulskis K, Sivachenko A, et al. Mutational heterogeneity in cancer and the search for new cancer-associated genes. Nature. 2013; 499:214-8.
Levine, Jacob H., Erin F. Simonds, Sean C. Bendall, Kara L. Davis, El-ad D. Amir, Michelle D. Tadmor, Oren Litvin, et al. 2015. «Data-Driven Phenotypic Dissection of AML Reveals Progenitor-like Cells That Correlate with Prognosis». Cell 162 (1): 184-97.
Matson, Vyara, Jessica Fessler, Riyue Bao, Tara Chongsuwat, Yuanyuan Zha, Maria-Luisa Alegre, Jason J. Luke, et Thomas F. Gajewski. 2018. «The Commensal Microbiome Is Associated with Anti-PD-1 Efficacy in Metastatic Melanoma Patients». Science 359 (6371): 104-8.
Mouton W, Compagnon C, Saker K, Daniel S, Lacoux X, Pozzetto B, et al. A novel whole-blood stimulation assay to detect and quantify memory T-cells in COVID-19 patients [Internet]. Infectious Diseases (except HIV/AIDS); 2021 March
Mulvey, M. A., J. D. Schilling, et S. J. Hultgren. 2001. «Establishment of a Persistent Escherichia Coli Reservoir during the Acute Phase of a Bladder Infection». Infection and Immunity 69 (7): 4572-79.
Necchi, Andrea, Andrea Anichini, Daniele Raggi, Alberto Briganti, Simona Massa, Roberta Lucianò, Maurizio Colecchia, et al. 2018. «Pembrolizumab as Neoadjuvant Therapy Before Radical Cystectomy in Patients With Muscle-Invasive Urothelial Bladder Carcinoma (PURE-01): An Open-Label, Single-Arm, Phase II Study». Journal of Clinical Oncology: Official Journal of the American Society of Clinical Oncology 36 (34): 3353-60.
Nejman, Deborah, Ilana Livyatan, Garold Fuks, Nancy Gavert, Yaara Zwang, Leore T. Geller, Aviva Rotter-Maskowitz, et al. 2020. «The Human Tumor Microbiome Is Composed of Tumor Type-Specific Intracellular Bacteria». Science 368 (6494): 973-80.
Noël, Grégory, Mireille Langouo Fontsa, Soizic Garaud, Pushpamali De Silva, Alexandre de Wind, Gert G. Van den Eynden, Roberto Salgado, et al. 2021. «Functional Th1-Oriented T Follicular Helper Cells That Infiltrate Human Breast Cancer Promote Effective Adaptive Immunity». The Journal of Clinical Investigation, août, 139905.
Overacre-Delgoffe, Abigail E., Hannah J. Bumgarner, Anthony R. Cillo, Ansen H. P. Burr, Justin T. Tometich, Amrita Bhattacharjee, Tullia C. Bruno, Dario A. A. Vignali, et Timothy W. Hand. 2021. «Microbiota-Specific T Follicular Helper Cells Drive Tertiary Lymphoid Structures and Anti-Tumor Immunity against Colorectal Cancer». Immunity 54 (12): 2812-2824.e4.
Pardoll, Drew M. 2012. «The Blockade of Immune Checkpoints in Cancer Immunotherapy». Nature Reviews. Cancer 12 (4): 252-64.
Patnaik, Amita, S. Peter Kang, Drew Rasco, Kyriakos P. Papadopoulos, Jeroen Elassaiss-Schaap, Muralidhar Beeram, Ronald Drengler, et al. 2015. «Phase I Study of Pembrolizumab (MK-3475; Anti-PD-1 Monoclonal Antibody) in Patients with Advanced Solid Tumors». Clinical Cancer Research: An Official Journal of the American Association for Cancer Research 21 (19): 4286-93.
Petitprez, Florent, Aurélien de Reyniès, Emily Z. Keung, Tom Wei-Wu Chen, Cheng-Ming Sun, Julien Calderaro, Yung-Ming Jeng, et al. 2020. «B Cells Are Associated with Survival and Immunotherapy Response in Sarcoma». Nature 577 (7791): 556-60.
Pfister, C., G. Gravis, A. Flechon, C. M. Chevreau, H. Mahammedi, B. Laguerre, A. Guillot, et al. 2021. «652O Dose-Dense Methotrexate, Vinblastine, Doxorubicin and Cisplatin (Dd-MVAC) or Gemcitabine and Cisplatin (GC) as Perioperative Chemotherapy for Patients with Muscle-Invasive Bladder Cancer (MIBC): Results of the GETUG/AFU VESPER V05 Phase III Trial». Annals of Oncology 32 (septembre): S678.
Powles, Thomas, Ignacio Durán, Michiel S. van der Heijden, Yohann Loriot, Nicholas J. Vogelzang, Ugo De Giorgi, Stéphane Oudard, et al. 2018. «Atezolizumab versus Chemotherapy in Patients with Platinum-Treated Locally Advanced or Metastatic Urothelial Carcinoma (IMvigor211): A Multicentre, Open-Label, Phase 3 Randomised Controlled Trial». Lancet (London, England) 391 (10122): 748-57.
Powles, Thomas, Mark Kockx, Alejo Rodriguez-Vida, Ignacio Duran, Simon J. Crabb, Michiel S. Van Der Heijden, Bernadett Szabados, et al. 2019. «Clinical Efficacy and Biomarker Analysis of Neoadjuvant Atezolizumab in Operable Urothelial Carcinoma in the ABACUS Trial». Nature Medicine 25 (11): 1706-14.
Powles, Thomas, Se Hoon Park, Eric Voog, Claudia Caserta, Begoña P. Valderrama, Howard Gurney, Haralabos Kalofonos, et al. 2020. «Avelumab Maintenance Therapy for Advanced or Metastatic Urothelial Carcinoma». The New England Journal of Medicine 383 (13): 1218-30.
Pushalkar, Smruti, Mautin Hundeyin, Donnele Daley, Constantinos P. Zambirinis, Emma Kurz, Ankita Mishra, Navyatha Mohan, et al. 2018. «The Pancreatic Cancer Microbiome Promotes Oncogenesis by Induction of Innate and Adaptive Immune Suppression». Cancer Discovery 8 (4): 403-16.
Recondo G, Mahjoubi L, Maillard A, Loriot Y, Bigot L, Facchinetti F, et al. Feasibility and first reports of the MATCH-R repeated biopsy trial at Gustave Roussy. NPJ Precis Oncol. 2020; 4:27.
Ribas, Antoni, Daniel Sanghoon Shin, Jesse Zaretsky, Juliet Frederiksen, Andrew Cornish, Earl Avramis, Elizabeth Seja, et al. 2016. «PD-1 Blockade Expands Intratumoral Memory T Cells». Cancer Immunology Research 4 (3): 194-203.
Roberti, Maria Paula, Satoru Yonekura, Connie P. M. Duong, Marion Picard, Gladys Ferrere, Maryam Tidjani Alou, Conrad Rauber, et al. 2020. «Chemotherapy-Induced Ileal Crypt Apoptosis and the Ileal Microbiome Shape Immunosurveillance and Prognosis of Proximal Colon Cancer». Nature Medicine 26 (6): 919-31.
Routy, Bertrand, Emmanuelle Le Chatelier, Lisa Derosa, Connie P. M. Duong, Maryam Tidjani Alou, Romain Daillère, Aurélie Fluckiger, et al. 2018. «Gut Microbiome Influences Efficacy of PD-1-Based Immunotherapy against Epithelial Tumors». Science (New York, N.Y.) 359 (6371): 91-97.
Sade-Feldman, Moshe, Keren Yizhak, Stacey L. Bjorgaard, John P. Ray, Carl G. de Boer, Russell W. Jenkins, David J. Lieb, et al. 2018. «Defining T Cell States Associated with Response to Checkpoint Immunotherapy in Melanoma». Cell 175 (4): 998-1013.e20.
Sautès-Fridman, Catherine, Myriam Lawand, Nicolas A. Giraldo, Hélène Kaplon, Claire Germain, Wolf Herman Fridman, et Marie-Caroline Dieu-Nosjean. 2016. «Tertiary Lymphoid Structures in Cancers: Prognostic Value, Regulation, and Manipulation for Therapeutic Intervention». Frontiers in Immunology 7: 407.
Scapin, Giovanna, Xiaoyu Yang, Winifred W. Prosise, Mark McCoy, Paul Reichert, Jennifer M. Johnston, Ramesh S. Kashi, et Corey Strickland. 2015. «Structure of Full-Length Human Anti-PD1 Therapeutic IgG4 Antibody Pembrolizumab». Nature Structural & Molecular Biology 22 (12): 953-58.
Sepich-Poore, Gregory D., Laurence Zitvogel, Ravid Straussman, Jeff Hasty, Jennifer A. Wargo, et Rob Knight. 2021. «The Microbiome and Human Cancer». Science (New York, N.Y.) 371 (6536): eabc4552.
Sharma, Padmanee, Margitta Retz, Arlene Siefker-Radtke, Ari Baron, Andrea Necchi, Jens Bedke, Elizabeth R. Plimack, et al. 2017. «Nivolumab in Metastatic Urothelial Carcinoma after Platinum Therapy (CheckMate 275): A Multicentre, Single-Arm, Phase 2 Trial». The Lancet. Oncology 18 (3): 312-22.
Sivan, Ayelet, Leticia Corrales, Nathaniel Hubert, Jason B. Williams, Keston Aquino-Michaels, Zachary M. Earley, Franco W. Benyamin, et al. 2015. «Commensal Bifidobacterium Promotes Antitumor Immunity and Facilitates Anti-PD-L1 Efficacy». Science (New York, N.Y.) 350 (6264): 1084-89.
Szabados, Bernadett, Alejo Rodriguez-Vida, Ignacio Durán, Simon J. Crabb, Michiel S. Van Der Heijden, Albert Font Pous, Gwenaelle Gravis, et al. 2021. «Toxicity and Surgical Complication Rates of Neoadjuvant Atezolizumab in Patients with Muscle-Invasive Bladder Cancer Undergoing Radical Cystectomy: Updated Safety Results from the ABACUS Trial». European Urology Oncology 4 (3): 456-63.
Topalian, Suzanne L., Mario Sznol, David F. McDermott, Harriet M. Kluger, Richard D. Carvajal, William H. Sharfman, Julie R. Brahmer, et al. 2014. «Survival, Durable Tumor Remission, and Long-Term Safety in Patients with Advanced Melanoma Receiving Nivolumab». Journal of Clinical Oncology: Official Journal of the American Society of Clinical Oncology 32 (10): 1020-30.
Tsay, Jun-Chieh J., Benjamin G. Wu, Imran Sulaiman, Katherine Gershner, Rosemary Schluger, Yonghua Li, Ting-An Yie, et al. 2021. «Lower Airway Dysbiosis Affects Lung Cancer Progression». Cancer Discovery 11 (2): 293-307.
Tumeh, Paul C., Christina L. Harview, Jennifer H. Yearley, I. Peter Shintaku, Emma J. M. Taylor, Lidia Robert, Bartosz Chmielowski, et al. 2014. «PD-1 Blockade Induces Responses by Inhibiting Adaptive Immune Resistance». Nature 515 (7528): 568-71.
Vermeulen, S H, N Hanum, A J Grotenhuis, G Castaño-Vinyals, A G van der Heijden, K K Aben, I U Mysorekar, et L A Kiemeney. 2015. «Recurrent urinary tract infection and risk of bladder cancer in the Nijmegen bladder cancer study». British Journal of Cancer 112 (3): 594-600.
Vétizou, Marie, Jonathan M. Pitt, Romain Daillère, Patricia Lepage, Nadine Waldschmitt, Caroline Flament, Sylvie Rusakiewicz, et al. 2015. «Anticancer Immunotherapy by CTLA-4 Blockade Relies on the Gut Microbiota». Science (New York, N.Y.) 350 (6264): 1079-84.
Voabil, Paula, Marjolein de Bruijn, Lisanne M. Roelofsen, Sanne H. Hendriks, Simone Brokamp, Marlous van den Braber, Annegien Broeks, et al. 2021. «An Ex Vivo Tumor Fragment Platform to Dissect Response to PD-1 Blockade in Cancer». Nature Medicine 27 (7): 1250-61.
Wang, Minan, Li-Chin Yao, Mingshan Cheng, Danying Cai, Jan Martinek, Chong-Xian Pan, Wei Shi, et al. 2018. «Humanized Mice in Studying Efficacy and Mechanisms of PD-1—Targeted Cancer Immunotherapy». FASEB Journal: Official Publication of the Federation of American Societies for Experimental Biology 32 (3): 1537-49.
Wong, Yien Ning Sophia, Kroopa Joshi, Pramit Khetrapal, Mazlina Ismail, James L. Reading, Mariana Werner Sunderland, Andrew Georgiou, et al. 2018. «Urine-Derived Lymphocytes as a Non-Invasive Measure of the Bladder Tumor Immune Microenvironment». The Journal of Experimental Medicine 215 (11): 2748-59.
Wu, Jianxuan, Chunjing Bao, R. Lee Reinhardt, et Soman N. Abraham. 2021. «Local Induction of Bladder Th1 Responses to Combat Urinary Tract Infections». Proceedings of the National Academy of Sciences of the United States of America 118 (10): e2026461118.
Wu, Jianxuan, Yuxuan Miao, et Soman N. Abraham. 2017. «The Multiple Antibacterial Activities of the Bladder Epithelium». Annals of Translational Medicine 5 (2): 35.
Xu, Mo, Maria Pokrovskii, Yi Ding, Ren Yi, Christy Au, Oliver J. Harrison, Carolina Galan, Yasmine Belkaid, Richard Bonneau, et Dan R. Littman. 2018. «C-MAF-Dependent Regulatory T Cells Mediate Immunological Tolerance to a Gut Pathobiont». Nature 554 (7692): 373-77.

1. An in vitro method of determining if an individual having a muscle-invasive bladder cancer (MIBC) or a kidney cancer is likely to respond to an anti-PD1/PD-L1/PD-L2 Ab-based therapy, comprising a step of assessing, in a sample from said patient:
(i) the level of E. coli-specific IgG,
(ii) the level of S. capitis-specific IgG and IgA,
(iii) the level of BCG-specific IgG,
(iv) the level of E. coli-specific CXCL13 producing follicular helper CD4⁺ T (T_{FH}), and/or
(v) the level of E. coli-specific CXCL9 producing T cells,
wherein if said level(s) is(are) superior to (a) predetermined threshold(s), the individual is likely to respond to said anti-PD1/PD-L1/PD-L2 Ab-based therapy and the individual is treated with such an anti-PD1/PD-L1/PD-L2 Ab-based therapy.

2. The method of claim 1, wherein the level of Escherichia coli-specific T_{FH} is assessed by a method comprising:
(i) contacting peripheral blood cells from said individual with pasteurized E. coli, or peptides from an E. coli amino acid sequence, in appropriate conditions to stimulate said cells, and
(ii) following cell stimulation, measuring the expression of CXCL13, CXCL9, IFNg and/or TNFa in the supernatant,
wherein if the level of CXCL13 or CXCL9 or IFNg or TNFa expression is superior to the negative control (unstimulated whole blood), the individual is likely to respond to the anti-PD1/PD-L1/PD-L2 Ab-based therapy.

3. The method of claim 1, wherein the level of Escherichia coli-specific T_{FH} is assessed by a method comprising:
(i) generating monocytes-derived dendritic cells (mo-DC) from PBMC from the individual;
(ii) incubating said mo-DC with a suspension of Escherichia coli;
(iii) adding an antibiotic;
(iv) co-culturing said mo-DC with memory CD4⁺ T cells from the individual during 1 to 3 days, preferably 2 days;
(v) measuring the expression of CXCL13 or IL-21 in the supernatant,
wherein if the level of CXCL13 or IL-21 expression is superior to the negative control (unstimulated cells), the individual is likely to respond to the anti-PD1/PD-L1/PD-L2 Ab-based therapy.

4-16. (canceled)

17. A method of treating muscle-invasive bladder cancer (MIBC) in a patient in need thereof, comprising administering an effective amount of an immunogenic composition comprising antigens from bacteria selected from the group consisting of Escherichia coli (E. coli), Staphylococcus capitis (S. capitis), Bacillus Calmette-Guérin (BCG) and mixtures thereof.

18. The method of claim 17, wherein said composition comprises antigens from uropathogenic E. coli (UPEC).

19. The method of claim 17, wherein said composition comprises E. coli and/or S. capitis and/or BCG bacteria or fragments thereof.

20. The method of claim 17, wherein said composition comprises E. coli and/or S. capitis proteins or peptides and at least one adjuvant.

21. The method of claim 17, wherein said composition comprises a nucleic acid encoding E. coli and/or S. capitis proteins.

22. The method of claim 17, wherein said composition is administered in combination with an anti-PD1/PD-L1/PD-L2 Ab-based therapy.

23. The method of claim 17, wherein said composition is administered via intravesical instillation or via oral administration of gastroresistant capsules.

24. The method of claim 17, wherein said composition is administered intramuscularly.

25. A method of treating muscle-invasive bladder cancer (MIBC) in a patient in need thereof, comprising administering an effective amount of an antibody targeting an antigen from E. coli or from S. capitis.

26. The method of claim 25, wherein said antibody is conjugated to a cytotoxic drug.

27. The method of claim 25, wherein said antibody is administered intravenously or via intravesical instillation.

28. The method of claim 26, wherein said antibody-drug conjugate is administered intravenously or via intravesical instillation.


SEQUENCE LISTING (28 sequences)

SEQ ID NO: 1 - protein, 9 aa, Escherichia coli
LLGGLLTMV

SEQ ID NO: 2 - protein, 15 aa, Escherichia coli
PRVVYNSRTDKPWPV

SEQ ID NO: 3 - protein, 15 aa, Escherichia coli
YNSRTDKPWPVALYL

SEQ ID NO: 4 - protein, 15 aa, Escherichia coli
TDKPWPVALYLTPVS

SEQ ID NO: 5 - protein, 15 aa, Escherichia coli
PWPVALYLTPVSSAG

SEQ ID NO: 6 - protein, 15 aa, Escherichia coli
ALYLTPVSSAGGVAI

SEQ ID NO: 7 - protein, 15 aa, Escherichia coli
AGSLIAVLILRQTNN

SEQ ID NO: 8 - protein, 15 aa, Escherichia coli
IAVLILRQTNNYNSD

SEQ ID NO: 9 - protein, 15 aa, Escherichia coli
ILRQTNNYNSDDFQF

SEQ ID NO: 10 - protein, 15 aa, Escherichia coli
PRVVYNSRTDKPWPV

SEQ ID NO: 11 - protein, 15 aa, Escherichia coli
YNSRTDKPWPVALYL

SEQ ID NO: 12 - protein, 15 aa, Escherichia coli
TDKPWPVALYLTPVS

SEQ ID NO: 13 - protein, 15 aa, Escherichia coli
PWPVALYLTPVSSAG

SEQ ID NO: 14 - protein, 15 aa, Escherichia coli
ALYLTPVSSAGGVAI

SEQ ID NO: 15 - protein, 15 aa, Escherichia coli
AGSLIAVLILRQTNN

SEQ ID NO: 16 - protein, 15 aa, Escherichia coli
IAVLILRQTNNYNSD

SEQ ID NO: 17 - protein, 15 aa, Escherichia coli
ILRQTNNYNSDDFQF

SEQ ID NO: 18 - protein, 15 aa, Escherichia coli
LVGQVYYRDESLRFY

SEQ ID NO: 19 - protein, 15 aa, Escherichia coli
FPTVNANKQATAFSS

SEQ ID NO: 20 - protein, 14 aa, Escherichia coli
NANKQATAFSSSQQ

SEQ ID NO: 21 - protein, 15 aa, Escherichia coli
SRLRHFLFNAGLLMH

SEQ ID NO: 22 - protein, 15 aa, Escherichia coli
GWRFTGDNLRTQIAA

SEQ ID NO: 23 - protein, 15 aa, Escherichia coli
TGDNLRTQIAAYYSL

SEQ ID NO: 24 - protein, 15 aa, Escherichia coli
LRTQIAAYYSLSNKS

SEQ ID NO: 25 - protein, 15 aa, Escherichia coli
IAAYYSLSNKSVERN

SEQ ID NO: 26 - protein, 15 aa, Escherichia coli
YSLSNKSVERNKDLT

SEQ ID NO: 27 - protein, 15 aa, Escherichia coli
NKSVERNKDLTISVK

SEQ ID NO: 28 - protein, 15 aa, Escherichia coli
ERNKDLTISVKDDRR